SEQUENCE LISTING (3 sequences)

SEQ ID NO: 1 - DNA, 2500 bp, Homo sapiens
  source 1..2500: mol_type = genomic DNA
tatccaagagctatcagcactcccatgtttattgtagcactgttcacaatagccaagatttggaagtactctaagtgtccattagcagatgaatggataaagacaatgtggtaatacacataatggagtactattcagtcataaagaagaattagatcctgtcatttgcaataacatggatggaactggaggtcataatgttgagtgaaataaaccaggcacagaaagacaaactttgcatgttctcacttatttatgggagctaaaaactaaaataactgaactcacagagatagagagtagaaggatggttacgagaggatgggaagggtagcgaggtgggtaggggggatgtggggatcattaatgggtataaaaaatagttagaggccaggcgcagtggctcacgcctgtaatcccagcactttgggaggccgaggtaggcggaacacctgaggagttcaagaccagcctggccaatatgatgaaaccccgtctctactaaaaatacaaaaattagctgggcgtgatggtgtgcacctgtagtcccagctgcttgggaggctgaggcaggagaatcgctggaacccaagaggtgaaggttgcagtgagctgagatcgcgtcactgcactccagcctgggtgacagagtgagactccacatcaaaaaaaaaaaaaaaaagttagaaagattgaataagacctaatatttgctagcacaacagggtgaatatagtaaaaaataatttatttgtaccttcaaaaataactagacaagtataattgggttgtttgtaacacacaaaaaataagtacttgaagtggtggataccccatttaccctgatgtgattattttgtattgcaggcctctatcagaatatctcatgtaacccataaatatatacacctactctgtacccacaaaaagtttttaaaaagaaaaataaatagcaaccgaaaaaaaaagagagggagaaaagaaaaaagaaaaaaaaatcaagtgcctggctgggtagaataaattctaaggccacaatgttactgaccatgggttttttggctctcagtgtatagaaattgacacaaggccaatagtcttcccaaacatgctttactggaacttacgccctggcataagggccacaacaaaagagagagcgaattctctggcttgctgactccttggaaaaaaccggtagggatttttttattaggcaaagcacaggaattgacgtcagaggcaggatgtgctgctgggcaaagcatacgagaagtggggtatgcaggtcagcattacttggttgcaatggttatcttgaggaatgggccaactggtggtctggccagtggcaacaaggctgtaaatcaattattcagcattccttcccaaggtgggacacccggcaacattgtttatctcctaaggccagttcctggaattaagtgaaaggatgactaatggacatgttgtcagtgaggtagtggtgtgggttttgtgaccagtgggaatgcacgaaagaatgctttagcggggagtgagctgaagccaagccccatccctactctgtctcaaagtgagttcagaaaaggggatttaaagaattctttttttttttttttttttttttgagacagagtcttgctctgtcgcccaggctggagtgcagtggcgccatcttggctcactgcaagctccgccccccgggttcatgccattctcctgcctcagcctcccaagtagctgggactgcaggtgcctaccaccaagcccagctaattttttgtattttttttttagtagagacggggtttcaccatgttagccaggatggtctcgatctcctgacctcgtgatctgcccgccttagcctcccaaagtgctgggattacaggcatgagcctccgcccccggccttaaataattcttaaaggaagtaaagttaactttgaaagaactatcaggatttggattgactgaaaggagtggggaagcttagggaggaggtgcttgccagacactgggtcatggcagtggtcggtgaagctgcagttgcctagggcagggatggagagagagtctgggcatgaggagagggtctcgggatgtttggctggactagattttacagaaagccttatccaggcttttaaaattactctttccagacttcatctgagactccttcttcagccaacattccttagccctgaatacatttcctatcctcatctttcccttcttttttttcctttcttttacatgtttaaatttaaaccattcttcgtgaccccttttcttgggagattcatggcaagaacgagaagaatgatggtgcttgttaggggatgtcctgtctctctgaactttggggtcctatgcattaaataattttcctgacgagctcaagtgctccctctggtctacaatccctggcggctggccttcatcccttgggcaagcattgcatacagctcatggccctccctctaccataccc

SEQ ID NO: 2 - DNA, 21 bp, synthetic construct
  source 1..21: mol_type = other DNA
gttcttcatctgcaccataaa

SEQ ID NO: 3 - DNA, 64 bp, synthetic construct
  source 1..64: mol_type = other DNA
gatccgggatgagtccatcaaggaagattcaagagatcttccttgatggactcatccttttttg

(19) United States
(12) Patent Application Publication
(10) Pub. No.: US 2025/0282774 A1
(43) Pub. Date: Sep. 11, 2025

(54) FUSED RING COMPOUND

(71) Applicant: SHANGHAI SHIJIANG BIOTECHNOLOGY CO., LTD., Shanghao (CN)

(72) Inventors: Yufeng SHI, Nanjing City (CN); Wenjiang MA, Nanjing City (CN)

(73) Assignee: SHANGHAI SHIJIANG BIOTECHNOLOGY CO., LTD., Shanghao (CN)

(21) Appl. No.: 18/862,584

(22) PCT Filed: May 5, 2023
(86) PCT No.: PCT/CN2023/092263

§ 371 Date: Nov. 4, 2024

(30) Foreign Application Priority Data
May 5, 2022 (CN) 202210481046.4
May 5, 2022 (CN) 202210481049.8

(51) Int. Cl. C07D 455/08; A61K 31/4375; C07D 519/00

(52) U.S. Cl. CPC C07D 455/08; A61K 31/4375; C07D 519/00

(57) ABSTRACT

The present invention relates to a fused ring compound; specifically, provided in the present invention is the use of a compound of formula I, or an optical isomer thereof, or a racemate thereof, or a solvate thereof, or a pharmaceutically acceptable salt thereof, or a deuterated compound thereof, in the preparation of a composition or a formulation for the prevention and/or treatment of tumours. The compound of the present invention has a significant and excellent precise therapeutic effect on tumours with low expression or no expression and low activity or no activity of mitochondrial permeability transition pores, low expression or no expression and low activity or no activity of peptidyl prolyl isomerase F, low expression or no expression of NNMT genes, high expression of DNA methylase, high expression of UHRFI, high methylation levels of NNMT gene nucleotide sites, and/or high methylation levels of DNA CpG sites in the NNMT gene region.

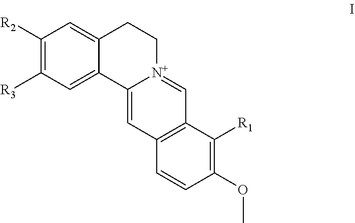

### FIELD OF THE INVENTION

The present invention relates to the field of medicine. Specifically, the present invention relates to fused ring compound.

### BACKGROUND TECHNOLOGY

Tumor is a common disease that seriously endangers human health, and the mortality rate of malignant tumors is rising. Due to the heterogeneity of tumor, simply using the same treatment method or medication based on source or pathological characteristic of tumors can easily lead to improper treatment, which can delay valuable treatment time and opportunities for patient. Therefore, it is very necessary to take precision treatment according to the different features of tumor. With the development of biological technology, tumors are typed at the molecular level such as gene and protein level, etc, and more and more changes in tumor-related gene and the expression and activity of protein have been discovered, changes in tumor-related gene and the expression and activity of protein play an important role in the development of malignant tumors, the discovery and application of biomarkers can provide precise guidance for the application of related drug and make precision treatment of tumor possible, thereby achieving targeted administration of drug, significantly improving treatment effect, reducing the dose of drug and reducing toxic side effects.

Therefore, there is an urgent need in the art to develop a drug that can achieve precision treatment on tumor.

### SUMMARY OF THE INVENTION

The present invention provide a compound, the compound has excellent precision treatment effect on tumor with low expression, no expression, low activity or no activity of mitochondria permeability transition pore, low expression, no expression, low activity or no activity of peptidyl-prolyl cis-trans isomerase F, low or no expression of NNMT gene, high expression of DNA methylase, high expression of UHRF1, high methylation level of nucleotide site of NNMT gene, and/or high methylation level of DNA CpG site of NNMT gene.

In the first aspect of the present invention, it provides a compound of formula I, or an optical isomer thereof, or a racemate thereof, or a solvate thereof, or a pharmaceutically acceptable salt thereof, or a deuterated compound thereof;

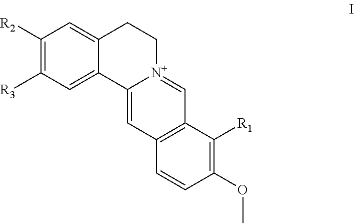

wherein,
R₁ is substituted or unsubstituted 3-12 membered heterocycloalkyl;
R₂ and R₃, together with the carbon atoms to which they are connected, form substituted or unsubstituted 3-12 membered heterocycloalkane ring.

In another preferred embodiment, each “substituted” means that one or more (preferably 1, 2, 3, 4, 5, 6, 7, or 8) hydrogen atoms on the ring or group are independently substituted by substituent selected from the group consisting of C1-C10 alkyl, C3-C8 cycloalkyl, C1-C10 haloalkyl, C3-C8 halocycloalkyl, C3-C8 cycloalkoxyl, C3-C8 cycloalkylthio, C3-C8 halocycloalkoxyl, C3-C8 halocycloalkylthio, halogen, nitro, —CN, hydroxyl, sulfhydryl, amino, C1-C4 carboxyl, C2-C8 ester group, C2-C4 amide group, C1-C10 alkoxyl, C1-C10 alkylthio, C1-C10 haloalkoxyl, C1-C10 haloalkylthio, C6-C12 aryl, 5-12 membered heteroaryl, 3-8 membered heterocycloalkyl.

In another preferred embodiment, each “substituted” means that one or more (preferably 1, 2, 3, 4, 5, 6, 7, or 8) hydrogen atoms on the ring or group are independently substituted by substituent selected from the group consisting of C1-C8 alkyl, C3-C8 cycloalkyl, C1-C8 haloalkyl, C3-C8 halocycloalkyl, C3-C8 cycloalkoxyl, C3-C8 cycloalkylthio, C3-C8 halocycloalkoxyl, C3-C8 halocycloalkylthio, halogen, nitro, —CN, hydroxyl, sulfhydryl, amino, C1-C4 carboxyl, C2-C8 ester group, C2-C4 amide group, C1-C8 alkoxyl, C1-C8 alkylthio, C1-C8 haloalkoxyl, C1-C8 haloalkylthio, C6-C10 aryl, 5-10 membered heteroaryl, 3-8 membered heterocycloalkyl.

In another preferred embodiment, each “substituted” means that one or more (preferably 1, 2, 3, 4, 5, 6, 7, or 8) hydrogen atoms on the ring or group are independently substituted by substituent selected from the group consisting of C1-C6 alkyl, C3-C8 cycloalkyl, C1-C6 haloalkyl, C3-C8 halocycloalkyl, C3-C8 cycloalkoxyl, C3-C8 cycloalkylthio, C3-C8 halocycloalkoxyl, C3-C8 halocycloalkylthio, halogen, nitro, —CN, hydroxyl, sulfhydryl, amino, C1-C4 carboxyl, C2-C6 ester group, C2-C4 amide group, C1-C6 alkoxyl, C1-C6 alkylthio, C1-C6 haloalkoxyl, C1-C6 haloalkylthio, C6-C10 aryl, 5-10 membered heteroaryl, 3-5 membered heterocycloalkyl.

In another preferred embodiment, each “substituted” means that one or more (preferably 1, 2, 3, 4, 5, 6, 7, or 8) hydrogen atoms on the ring or group are independently substituted by substituent selected from the group consisting of C1-C4 alkyl, C3-C8 cycloalkyl, C1-C4 haloalkyl, C3-C8 halocycloalkyl, C3-C8 cycloalkoxyl, C3-C8 cycloalkylthio, C3-C8 halocycloalkoxyl, C3-C8 halocycloalkylthio, halogen, nitro, —CN, hydroxyl, sulfhydryl, amino, C1-C4 carboxyl, C2-C6 ester group, C2-C4 amide group, C1-C4 alkoxyl, C1-C4 alkylthio, C1-C4 haloalkoxyl, C1-C4 haloalkylthio, C6-C10 aryl, 5-10 membered heteroaryl, 3-5 membered heterocycloalkyl.

In another preferred embodiment, the heterocyclic ring of the heterocycloalkyl, heteroaryl and heterocycloalkane ring has 1-4 (preferably 1, 2, 3 or 4) heteroatoms independently selected from the group consisting of N, O and S.

In another preferred embodiment, the heterocyclic ring of the heterocycloalkyl has 1-4 (preferably 1, 2, 3 or 4) heteroatoms independently selected from the group consisting of N, O and S.

In another preferred embodiment, the heterocyclic ring of the heteroaryl has 1-4 (preferably 1, 2, 3 or 4) heteroatoms independently selected from the group consisting of N, O and S.

In another preferred embodiment, the heterocyclic ring of the heterocycloalkane ring has 1-4 (preferably 1, 2, 3 or 4) heteroatoms independently selected from the group consisting of N, O and S.

In another preferred embodiment, each “substituted” means that one or more (preferably 1, 2, 3, 4, 5, 6, 7 or 8) hydrogen atoms on the ring or group are each independently substituted by a substituent.

In another preferred embodiment, the heterocycloalkyl has 0, 1 or 2 C═C ring double bonds.

In another preferred embodiment, the heterocycloalkane ring has 0, 1 or 2 C═C ring double bonds.

In another preferred embodiment, R₁ is substituted or unsubstituted 3 membered heterocycloalkyl, substituted or unsubstituted 4 membered heterocycloalkyl, substituted or unsubstituted 5 membered heterocycloalkyl, substituted or unsubstituted 6 membered heterocycloalkyl, substituted or unsubstituted 7 membered heterocycloalkyl, substituted or unsubstituted 8 membered heterocycloalkyl, substituted or unsubstituted 9 membered heterocycloalkyl, substituted or unsubstituted 10 membered heterocycloalkyl, substituted or unsubstituted 11 membered heterocycloalkyl, substituted or unsubstituted 12 membered heterocycloalkyl.

In another preferred embodiment, R₁ is substituted or unsubstituted piperazinyl, substituted or unsubstituted piperidinyl, substituted or unsubstituted morpholinyl, substituted or unsubstituted tetrahydropyrrolyl, substituted or unsubstituted piperidinospirooxetane group, substituted or unsubstituted hexamethyleneimine group.

In another preferred embodiment, the linking site of the heterocycloalkyl is located on the ring heteroatom of the heterocycloalkyl.

In another preferred embodiment, the linking site of the piperazinyl is located on the ring N atom of the piperazinyl.

In another preferred embodiment, the linking site of the piperidinyl is located on the ring N atom of the piperidinyl.

In another preferred embodiment, the linking site of the morpholinyl is located on the ring N atom of the morpholinyl.

In another preferred embodiment, the linking site of the tetrahydropyrrolyl is located on the ring N atom of the tetrahydropyrrolyl.

In another preferred embodiment, the linking site of the piperidinospirooxetane group is located on the ring N atom of the piperidinospirooxetane group.

In another preferred embodiment, the linking site of the hexamethyleneimine group is located on the ring N atom of the hexamethyleneimine group.

In another preferred embodiment, R₁ is

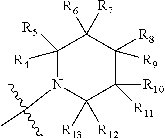

wherein,
R₄, R₅, R₆, R₇, R₈, R₉, R₁₀, R₁₁, R₁₂ and R₁₃ are each independently hydrogen, C1-C10 alkyl, C1-C10 haloalkyl, C1-C10 haloalkoxyl, C1-C10 haloalkylthio, halogen.

In another preferred embodiment, R₄, R₅, R₆, R₇, R₈, R₉, R₁₀, R₁₁, R₁₂ and R₁₃ are each independently hydrogen, C1-C8 alkyl, C1-C8 haloalkyl, C1-C8 haloalkoxyl, C1-C8 haloalkylthio, halogen.

In another preferred embodiment, R₄, R₅, R₆, R₇, R₈, R₉, R₁₀, R₁₁, R₁₂ and R₁₃ are each independently hydrogen, C1-C6 alkyl, C1-C6 haloalkyl, C1-C6 haloalkoxyl, C1-C6 haloalkylthio, halogen.

In another preferred embodiment, R₄, R₅, R₆, R₇, R₈, R₉, R₁₀, R₁₁, R₁₂ and R₁₃ are each independently hydrogen, C1-C4 alkyl, C1-C4 haloalkyl, C1-C4 haloalkoxyl, C1-C4 haloalkylthio, halogen.

In another preferred embodiment, R₄, R₅, R₆, R₇, R₈, R₉, R₁₀, R₁₁, R₁₂ and R₁₃ are each independently hydrogen, methyl, trifluoromethyl, trifluoromethoxyl, halogen (e.g, fluorine, chlorine).

In another preferred embodiment, R₁ is

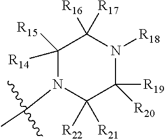

wherein,
R₁₄, R₁₅, R₁₆, R₁₇, R₁₈, R₁₉, R₂₀, R₂₁ and R₂₂ are each independently hydrogen, C1-C10 alkyl, C1-C10 haloalkyl, C1-C10 haloalkoxyl, C1-C10 haloalkylthio, halogen.

In another preferred embodiment, R₁₄, R₁₅, R₁₆, R₁₇, R₁₈, R₁₉, R₂₀, R₂₁ and R₂₂ are each independently hydrogen, C1-C8 alkyl, C1-C8 haloalkyl, C1-C8 haloalkoxyl, C1-C8 haloalkylthio, halogen.

In another preferred embodiment, R₁₄, R₁₅, R₁₆, R₁₇, R₁₈, R₁₉, R₂₀, R₂₁ and R₂₂ are each independently hydrogen, C1-C6 alkyl, C1-C6 haloalkyl, C1-C6 haloalkoxyl, C1-C6 haloalkylthio, halogen.

In another preferred embodiment, R₁₄, R₁₅, R₁₆, R₁₇, R₁₈, R₁₉, R₂₀, R₂₁ and R₂₂ are each independently hydrogen, C1-C4 alkyl, C1-C4 haloalkyl, C1-C4 haloalkoxyl, C1-C4 haloalkylthio, halogen.

In another preferred embodiment, R₁₄, R₁₅, R₁₆, R₁₇, R₁₈, R₁₉, R₂₀, R₂₁ and R₂₂ are each independently hydrogen, methyl, trifluoromethyl, trifluoromethoxyl, halogen (e.g, chlorine).

In another preferred embodiment, R₁ is

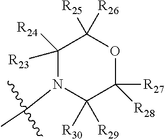

wherein,
R₂₃, R₂₄, R₂₅, R₂₆, R₂₇, R₂₈, R₂₉ and R₃₀ are each independently hydrogen, C1-C10 alkyl.

In another preferred embodiment, R₂₃, R₂₄, R₂₅, R₂₆, R₂₇, R₂₈, R₂₉ and R₃₀ are each independently hydrogen, methyl.

In another preferred embodiment, substituted or unsubstituted tetrahydropyrrolyl is

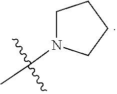 [SMILES: *N1CCCC1]

In another preferred embodiment, substituted or unsubstituted piperidinospirooxetane group is substituted or unsubstituted 1-oxo-7-azaspiro[3,5]nonyl.

In another preferred embodiment, substituted or unsubstituted piperidinospirooxetane group is

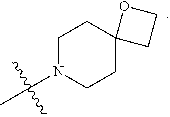 [SMILES: *N1CCC2(CCO2)CC1]

In another preferred embodiment, substituted or unsubstituted hexamethyleneimine group is

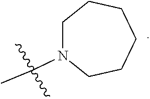 [SMILES: *N1CCCCCC1]

In another preferred embodiment, R₂ and R₃, together with the carbon atoms to which they are connected, form substituted or unsubstituted 3 membered heterocycloalkane ring, substituted or unsubstituted 4 membered heterocycloalkane ring, substituted or unsubstituted 5 membered heterocycloalkane ring, substituted or unsubstituted 6 membered heterocycloalkane ring, substituted or unsubstituted 7 membered heterocycloalkane ring, substituted or unsubstituted 8 membered heterocycloalkane ring, substituted or unsubstituted 9 membered heterocycloalkane ring, substituted or unsubstituted 10 membered heterocycloalkane ring, substituted or unsubstituted 11 membered heterocycloalkane ring, substituted or unsubstituted 12 membered heterocycloalkane ring.

In another preferred embodiment, R₂ and R₃, together with the carbon atoms to which they are connected, form dioxole, dihydro-dioxine.

In another preferred embodiment, the dioxole is 1, 3-dioxole.

In another preferred embodiment, the dihydro-dioxine is 2, 3-dihydro-1, 4-dioxine.

In another preferred embodiment, R₂ and R₃, together with the carbon atoms to which they are connected, form

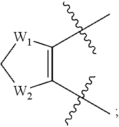

wherein, W₁ and W₂ are each independently O or S.

In another preferred embodiment, W₁ is O or S.

In another preferred embodiment, W₂ is O or S.

In another preferred embodiment, R₂ and R₃, together with the carbon atoms to which they are connected, form

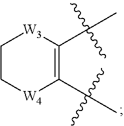

wherein, W₃ and W₃ are each independently O or S.

In another preferred embodiment, W₃ is O or S.

In another preferred embodiment, W₄ is O or S.

In another preferred embodiment, the halogen is F, Cl, Br or I.

In another preferred embodiment, the halo is mono-halo, di-halo, tri-halo or full-halo.

In another preferred embodiment, halo is fluoro, chloro, bromo, iodo.

In another preferred embodiment, the compound of formula I has the following structure of formula I-1:

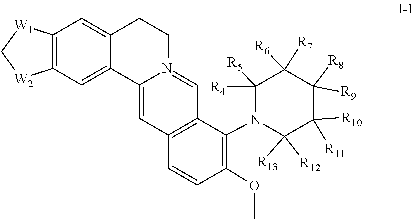

wherein, R₄, R₅, R₆, R₇, R₈, R₉, R₁₀, R₁₁, R₁₂, R₁₃, W₁ and W₂ are each independently defined as described above.

In another preferred embodiment, the compound of formula I has the following structure of formula I-2:

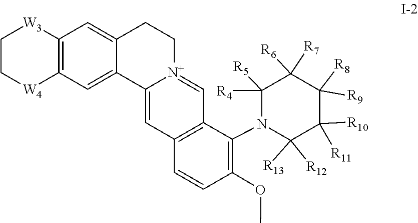

wherein, R₄, R₅, R₆, R₇, R₈, R₉, R₁₀, R₁₁, R₁₂, R₁₃, W₃ and W₄ are each independently defined as described above.

In another preferred embodiment, the compound of formula I has the following structure of formula I-3:

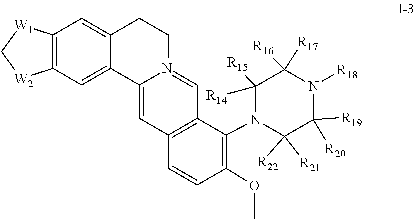

wherein, R₁₄, R₁₅, R₁₆, R₁₇, R₁₈, R₁₉, R₂₀, R₂₁, R₂₂, W₁ and W₂ are each independently defined as described above.

In another preferred embodiment, the compound of formula I has the following structure of formula I-4:

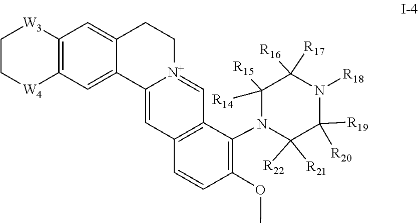

wherein, R₁₄, R₁₅, R₁₆, R₁₇, R₁₈, R₁₉, R₂₀, R₂₁, R₂₂, W₃ and W₄ are each independently defined as described above.

In another preferred embodiment, the compound of formula I has the following structure of formula I-5:

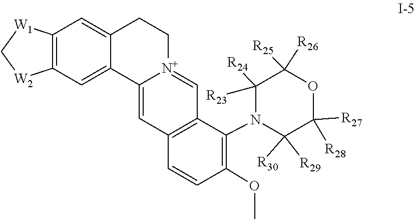

wherein, R₂₃, R₂₄, R₂₅, R₂₆, R₂₇, R₂₈, R₂₉, R₃₀, W₁ and W₂ are each independently defined as described above.

In another preferred embodiment, the compound of formula I has the following structure of formula I-6:

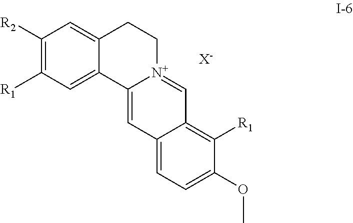

wherein, R₁, R₂ and R₃ are each independently defined as described above;
X⁻ is anionic salt radical.

In another preferred embodiment, the pharmaceutically acceptable salt of the compound of formula I comprises the salt formed by the compound of formula I and an acid.

In another preferred embodiment, the acid comprises one or more of hydrochloric acid, mucic acid, D-glucuronic acid, hydrobromic acid, hydrofluoric acid, hydroiodic acid, sulfuric acid, nitric acid, phosphoric acid, formic acid, acetic acid, trifluoroacetic acid, propionic acid, oxalic acid, malonic acid, succinic acid, fumaric acid, maleic acid, lactic acid, malic acid, tartaric acid, citric acid, picric acid, methanesulfonic acid, benzenemethanesulfonic acid, benzenesulfonic acid, trifluoro methanesulfonic acid, aspartic acid and glutamic acid.

In another preferred embodiment, the pharmaceutically acceptable salt of the compound of formula I comprises the salt formed by the compound of formula I and hydrochloric acid, mucic acid, D-glucuronic acid, hydrobromic acid, hydrofluoric acid, hydroiodic acid, sulfuric acid, nitric acid, phosphoric acid, formic acid, acetic acid, trifluoroacetic acid, propionic acid, oxalic acid, malonic acid, succinic acid, fumaric acid, maleic acid, lactic acid, malic acid, tartaric acid, citric acid, picric acid, methanesulfonic acid, benzenemethanesulfonic acid, benzenesulfonic acid, trifluoro methanesulfonic acid, aspartic acid or glutamic acid.

In another preferred embodiment, the salt radical of the pharmaceutically acceptable salt of the compound of formula I comprises the salt radical formed by the loss of a H⁺ in the acid.

In another preferred embodiment, the salt radical of the pharmaceutically acceptable salt of the compound of formula I comprises F⁻, Cl⁻, Br⁻, I⁻, HCOO⁻, CH₃COO⁻, SO₄²⁻, NO₃⁻ or

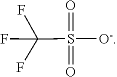

In another preferred embodiment, X⁻ is salt radical formed by the loss of a H⁺ in the acid.

In another preferred embodiment, X⁻ is F⁻, Cl⁻, Br⁻, I⁻, HCOO⁻, CH₃COO⁻, SO₄²⁻, NO₃⁻ or

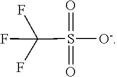

In another preferred embodiment, the compound of formula I, or an optical isomer thereof, or a racemate thereof, or a solvate thereof, or a pharmaceutically acceptable salt thereof, or a deuterated compound thereof is selected from the following group:

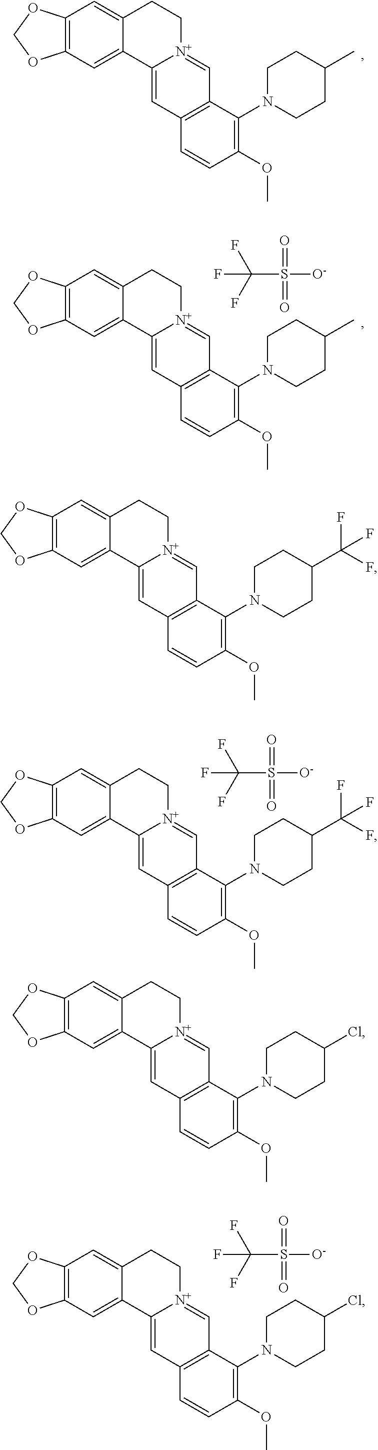

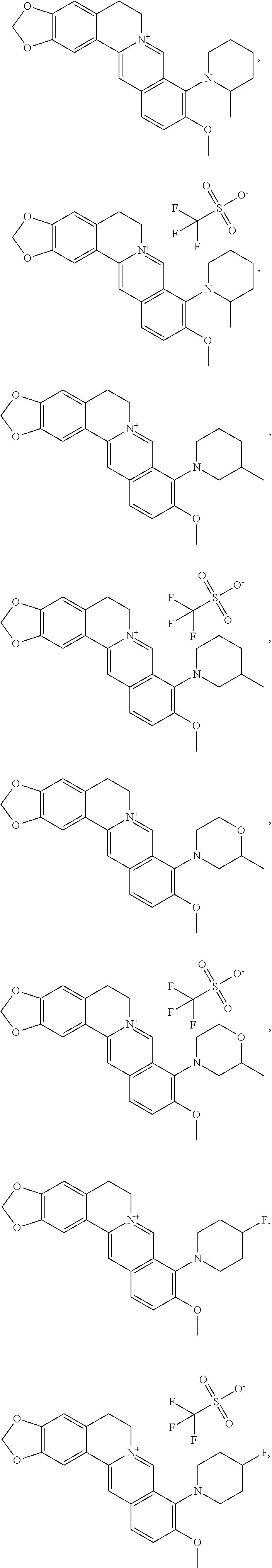

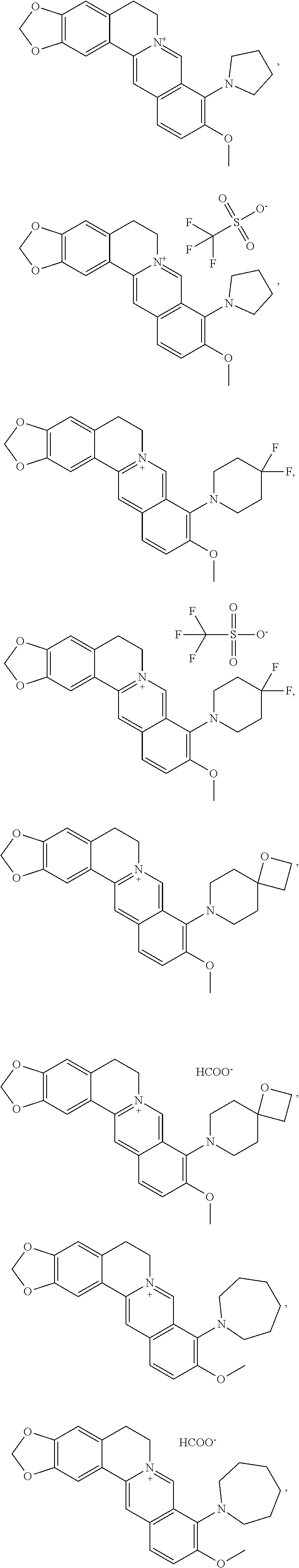

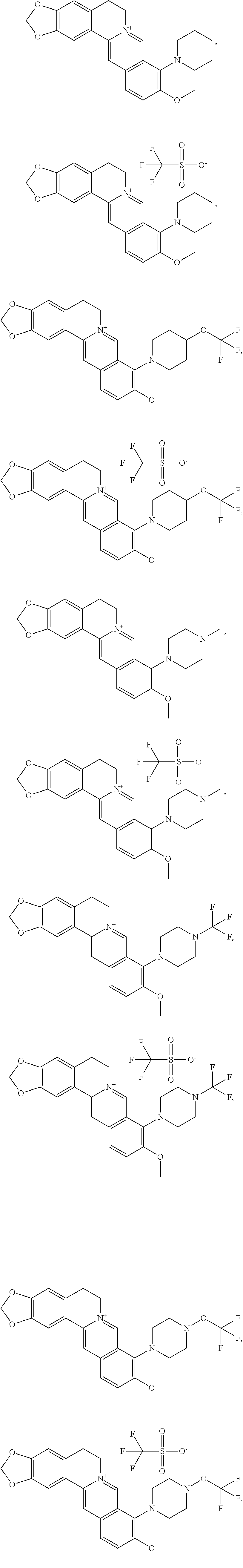

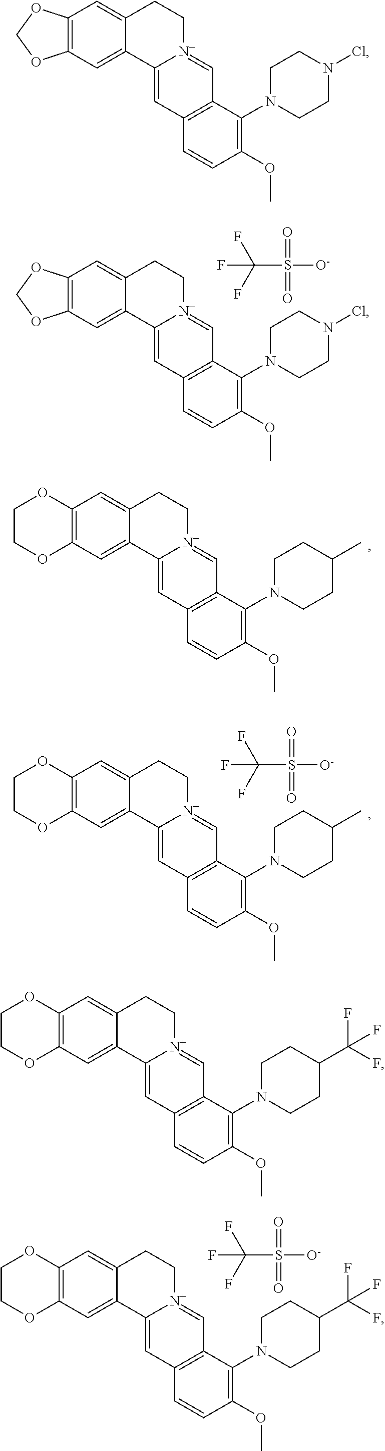

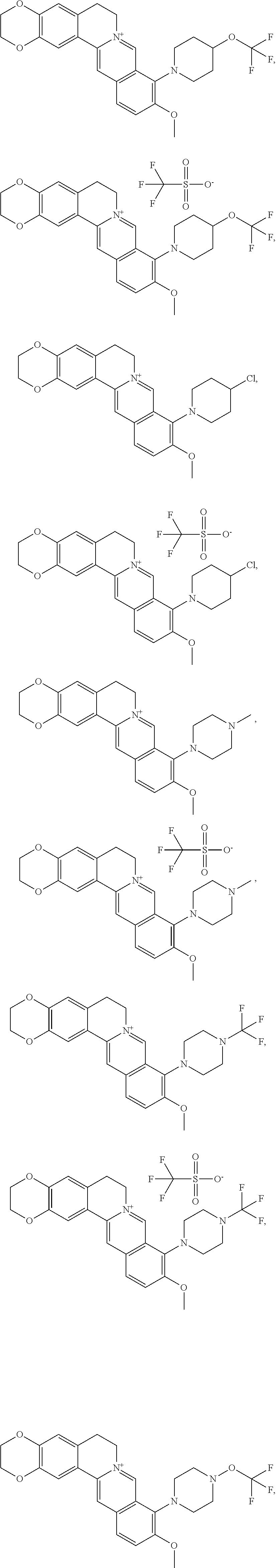

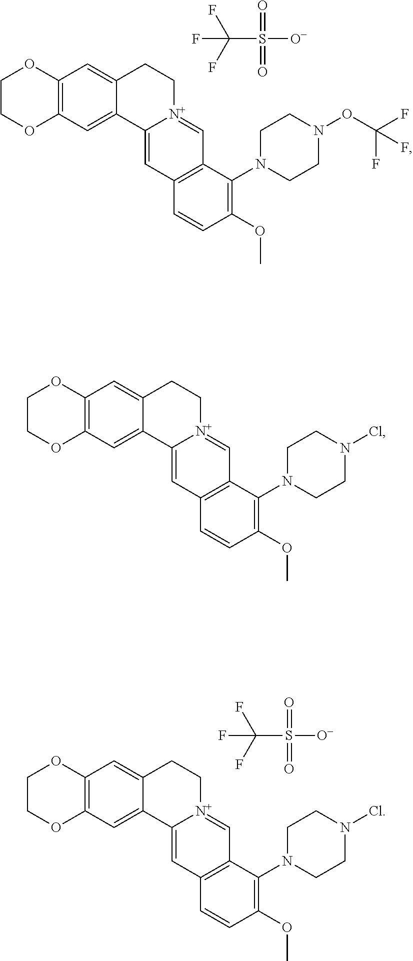 [SMILES: COc1ccc2cc3[n+](cc2c1N1CCN(OC(F)(F)F)CC1)CCc1cc2c(cc1-3)OCCO2 ; O=S(=O)([O-])C(F)(F)F ; COc1ccc2cc3[n+](cc2c1N1CCN(Cl)CC1)CCc1cc2c(cc1-3)OCCO2 ; COc1ccc2cc3[n+](cc2c1N1CCN(Cl)CC1)CCc1cc2c(cc1-3)OCCO2 ; O=S(=O)([O-])C(F)(F)F]

In the second aspect of the present invention, it provides a composition, the composition comprises (a) the compound of formula I, or an optical isomer thereof, or a racemate thereof, or a solvate thereof, or a pharmaceutically acceptable salt thereof, or a deuterated compound thereof according to the first aspect of the present invention.

In another preferred embodiment, the composition is pharmaceutical composition.

In another preferred embodiment, the composition further comprises (b) a pharmaceutically acceptable carrier.

In another preferred embodiment, the dosage form of the composition or preparation is a solid preparation, liquid preparation or semi-solid preparation.

In another preferred embodiment, the dosage form of the composition or preparation is oral preparation, external preparation or injection preparation.

In the third aspect of the present invention, it provides a use of the compound of formula I, or an optical isomer thereof, or a racemate thereof, or a solvate thereof, or a pharmaceutically acceptable salt thereof, or a deuterated compound thereof according to the first aspect of the present invention in the preparation of a composition or a preparation for preventing and/or treating tumor.

In another preferred embodiment, the tumor is human-derived tumor.

In another preferred embodiment, the tumor is human tumor.

In another preferred embodiment, the tumor comprises tumor with low expression, no expression, low activity or no activity of mitochondria permeability transition pore.

In another preferred embodiment, the tumor comprises tumor with low expression, no expression, low activity or no activity of peptidyl-prolyl cis-trans isomerase F.

In another preferred embodiment, the protein identification number of peptidyl-prolyl cis-trans isomerase F is UniProtKB/Swiss Prot: P30405, and the gene identification number of peptidyl-prolyl cis-trans isomerase F is NCBI Entrez Gene: 10105.

In another preferred embodiment, the tumor with low expression or low activity of mitochondria permeability transition pore means the expression level or activity level of mitochondria permeability transition pore in tumor cell is lower than the expression level or activity level of mitochondria permeability transition pore in the same type of cell or a normal cell.

In another preferred embodiment, the low expression or low activity of mitochondria permeability transition pore means the ratio (H1/H0) of the expression level or activity level H1 of mitochondria permeability transition pore in a cell (e.g. tumor cell) to the expression level or activity level H0 of mitochondria permeability transition pore in the same type of cell or a normal cell is <1.0, preferably ≤0.8, more preferably ≤0.7, more preferably ≤0.6, more preferably ≤0.5, more preferably ≤0.4, more preferably ≤0.3, more preferably ≤0.2, more preferably ≤0.1, more preferably ≤0.05, more preferably ≤0.01, more preferably ≤0.005, more preferably ≤0.001, more preferably ≤0.0001, more preferably ≤0.00001, more preferably ≤0.000001, more preferably ≤0.0000001.

In another preferred embodiment, the cell comprises tumor cell.

In another preferred embodiment, the same type of cell comprises the same type of tumor cell.

In another preferred embodiment, the same type of cell comprises the cell (e.g. the same type of tumor cell) with normal expression, high expression, normal activity or high activity of mitochondria permeability transition pore.

In another preferred embodiment, the same type of cell comprises the same type of cell (e.g. the same type of tumor cell) with normal expression, high expression, normal activity or high activity of mitochondria permeability transition pore

In another preferred embodiment, the normal cell comprises normal tissue cell (e.g. tumor origin cell, tumor-adjacent cell or para-tumor tissue cell).

In another preferred embodiment, the normal cell comprises normal tissue cell (e.g. tumor origin cell, tumor-adjacent cell or para-tumor tissue cell) with normal expression or normal activity of mitochondria permeability transition pore.

In another preferred embodiment, H0 refers to the expression level or activity level H0 of mitochondria permeability transition pore in the cell with normal expression, high expression, normal activity or high activity of mitochondria permeability transition pore.

In another preferred embodiment, the cell with normal expression, high expression, normal activity or high activity of mitochondria permeability transition pore comprises the cell that is not sensitive to the compound of formula I, or an optical isomer thereof, or a racemate thereof, or a solvate thereof, or a pharmaceutically acceptable salt thereof, or a deuterated compound thereof.

In another preferred embodiment, the tumor with low expression or low activity of peptidyl-prolyl cis-trans isomerase F means the expression level or activity level of peptidyl-prolyl cis-trans isomerase F in tumor cell is lower than the expression level or activity level of peptidyl-prolyl cis-trans isomerase F in the same type of cell or a normal cell.

In another preferred embodiment, the low expression or low activity of peptidyl-prolyl cis-trans isomerase F means the ratio (P1/P0) of the expression level or activity level P1 of peptidyl-prolyl cis-trans isomerase F in a cell (e.g. tumor cell) to the expression level or activity level P0 of peptidyl-prolyl cis-trans isomerase F in the same type of cell or a normal cell is <1.0, preferably ≤0.8, more preferably ≤0.7, more preferably ≤0.6, more preferably ≤0.5, more preferably ≤0.4, more preferably ≤0.3, more preferably ≤0.2, more preferably ≤0.1, more preferably ≤0.05, more preferably ≤0.01, more preferably ≤0.005, more preferably ≤0.001, more preferably ≤0.0001, more preferably ≤0.00001, more preferably ≤0.000001, more preferably ≤0.0000001.

In another preferred embodiment, the cell comprises the same type of cell.

In another preferred embodiment, the same type of cell comprises the same type of tumor cell.

In another preferred embodiment, the same type of cell comprises the cell (e.g. the same type of tumor cell) with normal expression, high expression, normal activity or high activity of peptidyl-prolyl cis-trans isomerase F.

In another preferred embodiment, the same type of cell comprises the same type of cell (e.g. the same type of tumor cell) with normal expression, high expression, normal activity or high activity of peptidyl-prolyl cis-trans isomerase F.

In another preferred embodiment, the normal cell comprises normal tissue cell (e.g. tumor origin cell, tumor-adjacent cell or para-tumor tissue cell).

In another preferred embodiment, the normal cell comprises normal tissue cell (e.g. tumor origin cell, tumor-adjacent cell or para-tumor tissue cell) with normal expression or normal activity of peptidyl-prolyl cis-trans isomerase F.

In another preferred embodiment, P0 refers to the expression level or activity level H0 of peptidyl-prolyl cis-trans isomerase F in the cell with normal expression, high expression, normal activity or high activity of peptidyl-prolyl cis-trans isomerase F.

In another preferred embodiment, the cell with normal expression, high expression, normal activity or high activity of peptidyl-prolyl cis-trans isomerase F comprises the cell that is not sensitive to the compound of formula I, or an optical isomer thereof, or a racemate thereof, or a solvate thereof, or a pharmaceutically acceptable salt thereof, or a deuterated compound thereof.

In another preferred embodiment, the mitochondria permeability transition pore inhibitor is administered to achieve low expression, no expression, low activity or no activity of mitochondria permeability transition pore in tumor.

In another preferred embodiment, the peptidyl-prolyl cis-trans isomerase F inhibitor is administered to achieve low expression, no expression, low activity or no activity of peptidyl-prolyl cis-trans isomerase F.

In another preferred embodiment, the inhibitor comprises specific inhibitor.

In another preferred embodiment, the mitochondria permeability transition pore inhibitor comprises inhibitor that can achieve low expression, no expression, low activity or no activity of mitochondria permeability transition pore in tumor.

In another preferred embodiment, the peptidyl-prolyl cis-trans isomerase F inhibitor comprises inhibitor that can achieve low expression, no expression, low activity or no activity of peptidyl-prolyl cis-trans isomerase F.

In another preferred embodiment, the mitochondria permeability transition pore inhibitor is selected from the group consisting of Cyclosporin A, CyP-D protein inhibitor, peroxide scavenger, and combinations thereof.

In another preferred embodiment, the peptidyl-prolyl cis-trans isomerase F inhibitor comprises shRNA.

In another preferred embodiment, the nucleotide sequence of shRNA is

GTTCTTCATCTGCACCATAAA.

In another preferred embodiment, the tumor comprises tumor with low or no expression of NNMT gene.

In another preferred embodiment, the tumor comprises tumor with high expression of DNA methylase.

In another preferred embodiment, the DNA methylase is selected from the group consisting of DNMT1, DNMT3a, DNMT3b, and combinations thereof.

In another preferred embodiment, the tumor comprises tumor with high expression of DNMT1.

In another preferred embodiment, the tumor comprises tumor with high expression of DNMT3a.

In another preferred embodiment, the tumor comprises tumor with high expression of DNMT3b.

In another preferred embodiment, the tumor comprises tumor with high expression of UHRF1.

In another preferred embodiment, the tumor comprises tumor with high methylation level of nucleotide site of NNMT gene.

In another preferred embodiment, the methylation of nucleotide site of NNMT gene comprises the methylation of cytosine nucleotide site of NNMT gene.

In another preferred embodiment, the methylation of nucleotide site of NNMT gene comprises the methylation of cytosine of NNMT gene.

In another preferred embodiment, the methylation of nucleotide site of NNMT gene comprises the methylation of the 5′carbon atom on the cytosine of the NNMT gene.

In another preferred embodiment, the tumor comprises tumor with high methylation level of DNA CpG site of NNMT gene.

In another preferred embodiment, the methylation of DNA CpG site of NNMT gene comprises the methylation of cytosine nucleotide site of DNA CpG site of NNMT gene.

In another preferred embodiment, the methylation of DNA CpG site of NNMT gene comprises the methylation of cytosine of DNA CpG site of NNMT gene.

In another preferred embodiment, the methylation of DNA CpG site of NNMT gene comprises the methylation of the 5′carbon atom on the cytosine of DNA CpG site of NNMT gene.

In another preferred embodiment, the NNMT gene is human-derived NNMT gene.

In another preferred embodiment, the NNMT gene is human NNMT gene.

In another preferred embodiment, the tumor with low or no expression of NNMT gene means that no NNMT protein can be detected in 1 μg of protein extracted from tumor using NNMT antibody, preferably in 5 μg of protein extracted from tumor, more preferably in 10 μg of protein extracted from tumor, more preferably in 100 μg of protein extracted from tumor, preferably in 1000 μg of protein extracted from tumor.

In another preferred embodiment, the tumor with low or no expression of NNMT gene means the expression level of NNMT gene in tumor cell is lower than that in the same type of cell or a normal cell.

In another preferred embodiment, the low or no expression of NNMT gene means the ratio (E1/E0) of the expression E1 of NNMT gene in a cell (e.g. tumor cell) to the expression E0 of NNMT gene in the same type of cell or a normal cell is <1.0, preferably ≤0.7, more preferably ≤0.6, more preferably ≤0.5, more preferably ≤0.4, more preferably ≤0.3, more preferably ≤0.2, more preferably ≤0.1, more preferably ≤0.05, more preferably ≤0.01, more preferably ≤0.005, more preferably ≤0.001, more preferably ≤0.0001, more preferably ≤0.00001, more preferably ≤0.000001, more preferably ≤0.0000001.

In another preferred embodiment, the cell comprises tumor cell.

In another preferred embodiment, the same type of cell comprises the same type of tumor cell.

In another preferred embodiment, the same type of cell comprises the cell (e.g. the same type of tumor cell) with normal or high expression of NNMT gene.

In another preferred embodiment, the same type of cell comprises the same type of cell with normal or high expression of NNMT gene.

In another preferred embodiment, the normal cell comprises normal tissue cell (e.g. tumor origin cell, tumor-adjacent cell or para-tumor tissue cell).

In another preferred embodiment, the normal cell comprises normal tissue cell (e.g. tumor origin cell, tumor-adjacent cell or para-tumor tissue cell) with normal expression of NNMT gene.

In another preferred embodiment, E0 refers to the expression level of NNMT gene in the cell with normal or high expression of NNMT gene.

In another preferred embodiment, the cell with normal or high expression of NNMT gene comprises the cell that is not sensitive to the compound of formula I, or an optical isomer thereof, or a racemate thereof, or a solvate thereof, or a pharmaceutically acceptable salt thereof, or a deuterated compound thereof.

In another preferred embodiment, the tumor with high expression of DNA methylase means that DNA methylase can be detected in 20 μg of protein extracted from tumor using DNA methylase antibody, preferably in 5 μg of protein extracted from tumor, more preferably in 1 μg of protein extracted from tumor, more preferably in 0.2 μg of protein extracted from tumor, more preferably in 0.05 μg of protein extracted from tumor, more preferably in 0.01 μg of protein extracted from tumor.

In another preferred embodiment, the tumor with high expression of DNA methylase means the expression level of DNA methylase in tumor cell is higher than that in the same type of cell or a normal cell.

In another preferred embodiment, the tumor with high expression of DNA methylase means the ratio (A1/A0) of the expression level A1 of DNA methylase in the tumor cell to the expression level A0 of DNA methylase in the same type of cell or a normal cell is >1.0, preferably ≥1.2, more preferably ≥1.5, more preferably ≥2, more preferably ≥3, more preferably ≥5, more preferably ≥8, more preferably ≥10, more preferably ≥15, more preferably ≥20, more preferably ≥30, more preferably ≥50, such as 2-50.

In another preferred embodiment, the same type of cell comprises the same type of tumor cell.

In another preferred embodiment, the same type of cell comprises the cell (e.g. the same type of tumor cell) with normal or low expression of DNA methylase.

In another preferred embodiment, the same type of cell comprises the same type of cell with normal or low expression of DNA methylase.

In another preferred embodiment, the normal cell comprises normal tissue cell (e.g. tumor origin cell, tumor-adjacent cell or para-tumor tissue cell).

In another preferred embodiment, the normal cell comprises normal tissue cell (e.g. tumor origin cell, tumor-adjacent cell or para-tumor tissue cell) with normal expression of DNA methylase.

In another preferred embodiment, A0 refers to the expression level of DNA methylase in the cell with normal or low expression of DNA methylase.

In another preferred embodiment, the cell with normal or low expression of DNA methylase comprises the cell that is not sensitive to the compound of formula I, or an optical isomer thereof, or a racemate thereof, or a solvate thereof, or a pharmaceutically acceptable salt thereof, or a deuterated compound thereof.

In another preferred embodiment, the tumor with high expression of DNMT1 means that DNMT1 protein can be detected in 20 μg of protein extracted from tumor using DNMT1 antibody, preferably in 5 μg of protein extracted from tumor, more preferably in 1 μg of protein extracted from tumor, more preferably in 0.2 μg of protein extracted from tumor, more preferably in 0.05 μg of protein extracted from tumor, more preferably in 0.01 μg of protein extracted from tumor.

In another preferred embodiment, the tumor with high expression of DNMT1 means the expression level of DNMT1 in tumor type of cell is higher than that in the same type of cell or a normal cell.

In another preferred embodiment, the tumor with high expression of DNMT1 means the ratio (B1/B0) of the expression level B1 of DNMT1 in the tumor cell to the expression level B0 of DNMT1 in the same type of cell or a normal cell is >1.0, preferably ≥1.2, more preferably ≥1.5, more preferably ≥2, more preferably ≥3, more preferably ≥5, more preferably ≥8, more preferably ≥10, more preferably ≥15, more preferably ≥20, more preferably ≥30, more preferably ≥50, such as 2-50.

In another preferred embodiment, the same type of cell comprises the same type of tumor cell.

In another preferred embodiment, the same type of cell comprises the cell (e.g. the same type of tumor cell) with normal or low expression of DNMT1.

In another preferred embodiment, the same type of cell comprises the same type of cell with normal or low expression of DNMT1.

In another preferred embodiment, the normal cell comprises normal tissue cell (e.g. tumor origin cell, tumor-adjacent cell or para-tumor tissue cell).

In another preferred embodiment, the normal cell comprises normal tissue cell (e.g. tumor origin cell, tumor-adjacent cell or para-tumor tissue cell) with normal expression of DNMT1.

In another preferred embodiment, B0 refers to the expression level of DNMT1 in the cell with normal or low expression of DNMT1.

In another preferred embodiment, the cell with normal or low expression of DNMT1 comprises the cell that is not sensitive to the compound of formula I, or an optical isomer thereof, or a racemate thereof, or a solvate thereof, or a pharmaceutically acceptable salt thereof, or a deuterated compound thereof.

In another preferred embodiment, the tumor with high expression of DNMT3a means that DNMT3a protein can be detected in 20 μg of protein extracted from tumor using DNMT3a antibody, preferably in 5 μg of protein extracted from tumor, more preferably in 1 μg of protein extracted from tumor, more preferably in 0.2 μg of protein extracted from tumor, more preferably in 0.05 μg of protein extracted from tumor, more preferably in 0.01 μg of protein extracted from tumor.

In another preferred embodiment, the tumor with high expression of DNMT3a means the expression level of DNMT3a in tumor cell is higher than that in the same type of cell or a normal cell.

In another preferred embodiment, the tumor with high expression of DNMT3a means the ratio (C1/C0) of the expression level C1 of DNMT3a in the tumor cell to the expression level C0 of DNMT3a in the same type of cell or a normal cell is >1.0, preferably ≥1.2, more preferably ≥1.5, more preferably ≥2, more preferably ≥3, more preferably ≥5, more preferably ≥8, more preferably ≥10, more preferably ≥15, more preferably ≥20, more preferably ≥30, more preferably ≥50, such as 2-50.

In another preferred embodiment, the same type of cell comprises the same type of tumor cell.

In another preferred embodiment, the same type of cell comprises the cell (e.g. the same type of tumor cell) with normal or low expression of DNMT3a.

In another preferred embodiment, the same type of cell comprises the same type of cell with normal or low expression of DNMT3a.

In another preferred embodiment, the normal cell comprises normal tissue cell (e.g. tumor origin cell, tumor-adjacent cell or para-tumor tissue cell).

In another preferred embodiment, the normal cell comprises normal tissue cell (e.g. tumor origin cell, tumor-adjacent cell or para-tumor tissue cell) with normal expression of DNMT3a.

In another preferred embodiment, C0 refers to the expression level of DNMT3a in the cell with normal or low expression of DNMT3a.

In another preferred embodiment, the cell with normal or low expression of DNMT3a comprises the cell that is not sensitive to the compound of formula I, or an optical isomer thereof, or a racemate thereof, or a solvate thereof, or a pharmaceutically acceptable salt thereof, or a deuterated compound thereof.

In another preferred embodiment, the tumor with high expression of DNMT3b means that DNMT3b protein can be detected in 20 μg of protein extracted from tumor using DNMT3b antibody, preferably in 5 μg of protein extracted from tumor, more preferably in 1 μg of protein extracted from tumor, more preferably in 0.2 μg of protein extracted from tumor, more preferably in 0.05 μg of protein extracted from tumor, more preferably in 0.01 μg of protein extracted from tumor.

In another preferred embodiment, the tumor with high expression of DNMT3b means the expression level of DNMT3b in tumor type of cell is higher than that in the same type of cell or a normal cell.

In another preferred embodiment, the tumor with high expression of DNMT3b means the ratio (D1/DO) of the expression level D1 of DNMT3b in the tumor cell to the expression level DO of DNMT3b in the same type of cell or a normal cell is >1.0, preferably ≥1.2, more preferably ≥1.5, more preferably ≥2, more preferably ≥3, more preferably ≥5, more preferably ≥8, more preferably ≥10, more preferably ≥15, more preferably ≥20, more preferably ≥30, more preferably ≥50, such as 2-50.

In another preferred embodiment, the same type of cell comprises the same type of tumor cell.

In another preferred embodiment, the same type of cell comprises the cell (e.g. the same type of tumor cell) with normal or low expression of DNMT3b.

In another preferred embodiment, the same type of cell comprises the same type of cell with normal or low expression of DNMT3b.

In another preferred embodiment, the normal cell comprises normal tissue cell (e.g. tumor origin cell, tumor-adjacent cell or para-tumor tissue cell).

In another preferred embodiment, the normal cell comprises normal tissue cell (e.g. tumor origin cell, tumor-adjacent cell or para-tumor tissue cell) with normal expression of DNMT3b

In another preferred embodiment, DO refers to the expression level of DNMT3b in the cell with normal or low expression of DNMT3b.

In another preferred embodiment, the cell with normal or low expression of DNMT3b comprises the cell that is not sensitive to the compound of formula I, or an optical isomer thereof, or a racemate thereof, or a solvate thereof, or a pharmaceutically acceptable salt thereof, or a deuterated compound thereof.

In another preferred embodiment, the tumor with high expression of UHRF1 means that UHRF1 protein can be detected in 20 μg of protein extracted from tumor using UHRF1 antibody, preferably in 5 μg of protein extracted from tumor, more preferably in 1 μg of protein extracted from tumor, more preferably in 0.2 μg of protein extracted from tumor, more preferably in 0.05 μg of protein extracted from tumor, more preferably in 0.01 μg of protein extracted from tumor.

In another preferred embodiment, the tumor with high expression of UHRF1 means the expression level of UHRF1 in tumor type of cell is higher than that in the same type of cell or a normal cell.

In another preferred embodiment, the tumor with high expression of UHRF1 means the ratio (F1/F0) of the expression level F1 of UHRF1 in the tumor cell to the expression level F0 of UHRF1 in the same type of cell or a normal cell is >1.0, preferably ≥1.2, more preferably ≥1.5, more preferably ≥2, more preferably ≥3, more preferably ≥5, more preferably ≥8, more preferably ≥10, more preferably ≥15, more preferably ≥20, more preferably ≥30, more preferably ≥50, such as 2-50.

In another preferred embodiment, the same type of cell comprises the same type of tumor cell.

In another preferred embodiment, the same type of cell comprises the cell (e.g. the same type of tumor cell) with normal or low expression of UHRF1.

In another preferred embodiment, the same type of cell comprises the same type of cell with normal or low expression of UHRF1.

In another preferred embodiment, the normal cell comprises normal tissue cell (e.g. tumor origin cell, tumor-adjacent cell or para-tumor tissue cell).

In another preferred embodiment, the normal cell comprises normal tissue cell (e.g. tumor origin cell, tumor-adjacent cell or para-tumor tissue cell) with normal expression of UHRF1 In another preferred embodiment, F0 refers to the expression level of UHRF1 in the cell with normal or low expression of UHRF1.

In another preferred embodiment, the cell with normal or low expression of UHRF1 comprises the cell that is not sensitive to the compound of formula I, or an optical isomer thereof, or a racemate thereof, or a solvate thereof, or a pharmaceutically acceptable salt thereof, or a deuterated compound thereof.

In another preferred embodiment, the high methylation level of nucleotide site of NNMT gene means the methylation level of nucleotide site of NNMT gene in a cell (e.g. tumor cell) is higher than that in the same type of cell or a normal cell.

In another preferred embodiment, the high methylation level of nucleotide site of NNMT gene means the ratio (L1/L0) of the methylation level L1 of nucleotide site of NNMT gene in a cell (e.g. tumor cell) to the methylation level L0 of nucleotide site of NNMT gene in the same type of cell or a normal cell is >1.0, preferably ≥1.2, more preferably ≥1.5, more preferably ≥2, more preferably ≥3, more preferably ≥5, more preferably ≥8, more preferably ≥10, more preferably ≥15, more preferably ≥20, more preferably ≥30, more preferably ≥50, such as 2-50.

In another preferred embodiment, the high methylation level of nucleotide site of NNMT gene means the methylation level of nucleotide site of NNMT gene in a cell (e.g. tumor cell) is ≥1%, more preferably ≥3%, more preferably ≥5%, more preferably ≥10%, more preferably ≥15%, more preferably ≥20%, more preferably ≥25%, more preferably ≥30%, more preferably ≥40%, more preferably ≥50%.

In another preferred embodiment, the cell comprises tumor cell.

In another preferred embodiment, the same type cell comprises the same type of tumor cell.

In another preferred embodiment, the same type of cell comprises the cell (e.g. the same type of tumor cell) with normal or low methylation level of nucleotide site of NNMT gene.

In another preferred embodiment, the same type of cell comprises the same type of cell with normal or low methylation level of nucleotide site of NNMT gene.

In another preferred embodiment, the normal cell comprises normal tissue cell (e.g. tumor origin cell, tumor-adjacent cell or para-tumor tissue cell).

In another preferred embodiment, the normal cell comprises normal tissue cell (e.g. tumor origin cell, tumor-adjacent cell or para-tumor tissue cell) with normal methylation level of nucleotide site of NNMT gene.

In another preferred embodiment, L0 refers to methylation level of nucleotide site of NNMT gene in the cell with normal or low methylation level of nucleotide site of NNMT gene.

In another preferred embodiment, the cell with normal or low methylation level of nucleotide site of NNMT gene comprises the cell that is not sensitive to the compound of formula I, or an optical isomer thereof, or a racemate thereof, or a solvate thereof, or a pharmaceutically acceptable salt thereof, or a deuterated compound thereof.

In another preferred embodiment, the high methylation level of nucleotide site of NNMT gene means the methylation level (M %) of nucleotide site of NNMT gene in a cell (e.g. tumor cell) is ≥3% and ≤M1%, wherein M1 is any positive integer from 3 to 100.

In another preferred embodiment, M1 is 5, 10, 15, 20, 25, 30, 35, 40, 45, 50, 55, 60, 65, 70, 80, 85, 90, 95 or 100.

In another preferred embodiment, the methylation level of nucleotide site of NNMT gene refers to the ratio of the number of methylated nucleotides to the number of all nucleotides in the NNMT gene.

In another preferred embodiment, the methylation level of nucleotide site of NNMT gene comprises the methylation level of nucleotide site in promoter region of NNMT gene.

In another preferred embodiment, the nucleotide sequence of the promoter region of NNMT gene is as shown in SEQ ID NO: 1.

In another preferred embodiment, the methylation level of nucleotide site of NNMT gene comprises the methylation level of nucleotide sites from 1050 bp before the transcription start site to 499 bp after the transcription start site in NNMT gene.

In another preferred embodiment, the sites from 1050 bp before the transcription start site to 499 bp after the transcription start site in NNMT gene is sites 951-2500 of nucleotide sequence as shown in SEQ ID NO: 1.

In another preferred embodiment, the methylation level of nucleotide site of NNMT gene comprises the methylation level of nucleotide sites from 1050 bp to 193 bp before the transcription start site in NNMT gene.

In another preferred embodiment, the sites from 1050 bp to 193 bp before the transcription start site in NNMT gene is sites 951-1808 of nucleotide sequence as shown in SEQ ID NO: 1.

In another preferred embodiment, the methylation level of nucleotide site of NNMT gene comprises the methylation level of nucleotide sites from 840 bp to 469 bp before the transcription start site in NNMT gene.

In another preferred embodiment, the sites from 840 bp to 469 bp before the transcription start site in NNMT gene is sites 1161-1532 of nucleotide sequence as shown in SEQ ID NO: 1.

In another preferred embodiment, the methylation level of nucleotide site of NNMT gene comprises the methylation level of nucleotide site between any two sites (including the two sites itself) selected from group consisting of site 114165695, site 114165730, site 114165769, site 114165804, site 114165938, site 114166050 and site 114166066 on human chromosome 11.

In another preferred embodiment, the methylation level of nucleotide site of NNMT gene comprises the methylation level of nucleotide sites selected from group consisting of site 114165695 on human chromosome 11, site 114165730 on human chromosome 11, site 114165769 on human chromosome 11, site 114165804 on human chromosome 11, site 114165938 on human chromosome 11, site 114166050 on human chromosome 11, site 114166066 on human chromosome 11, and combinations thereof.

In another preferred embodiment, the methylation level of nucleotide site of NNMT gene comprises the methylation level of nucleotide site between any two sites (including the two sites itself) selected from group consisting of site 1161, site 1196, site 1235, site 1270, site 1404, site 1516 and site 1532 in nucleotide sequence of SEQ ID NO: 1.

In another preferred embodiment, the methylation level of nucleotide site of NNMT gene comprises the methylation level of nucleotide sites selected from group consisting of site 1161 in SEQ ID NO: 1, site 1196 in SEQ ID NO: 1, site 1235 in SEQ ID NO: 1, site 1270 in SEQ ID NO: 1, site 1404 in SEQ ID NO: 1, site 1516 in SEQ ID NO: 1, site 1532 in SEQ ID NO: 1, and combinations thereof.

In another preferred embodiment, the high methylation level of DNA CpG site of NNMT gene means the methylation level of DNA CpG site of NNMT gene in a cell (e.g. tumor cell) is higher than that in the same type of cell or a normal cell.

In another preferred embodiment, the high methylation level of DNA CpG site of NNMT gene means the ratio (G1/G0) of the methylation level G1 of DNA CpG site of NNMT gene in a cell (e.g. tumor cell) to the methylation level G0 of DNA CpG site of NNMT gene in the same type of cell or a normal cell is >1.0, preferably ≥1.2, more preferably ≥1.5, more preferably ≥2, more preferably ≥3, more preferably ≥5, more preferably ≥8, more preferably ≥10, more preferably ≥15, more preferably ≥20, more preferably ≥30, more preferably ≥50, such as 2-50.

In another preferred embodiment, the high methylation level of DNA CpG site of NNMT gene means the methylation level of DNA CpG site of NNMT gene in a cell (e.g. tumor cell) is ≥1%, more preferably ≥3%, more preferably ≥5%, more preferably ≥10%, more preferably ≥15%, more preferably ≥20%, more preferably ≥25%, more preferably ≥30%, more preferably ≥40%, more preferably ≥50%.

In another preferred embodiment, the cell comprises tumor cell.

In another preferred embodiment, the same type of cell comprises the same type of tumor cell.

In another preferred embodiment, the same type of cell comprises the cell (e.g. the same type of tumor cell) with normal or low methylation level of DNA CpG site of NNMT gene.

In another preferred embodiment, the same type of cell comprises the same type of cell with normal or low methylation level of DNA CpG site of NNMT gene.

In another preferred embodiment, the normal cell comprises normal tissue cell (e.g. tumor origin cell, tumor-adjacent cell or para-tumor tissue cell).

In another preferred embodiment, the normal cell comprises normal tissue cell (e.g. tumor origin cell, tumor-adjacent cell or para-tumor tissue cell) with normal methylation level of DNA CpG site of NNMT gene.

In another preferred embodiment, G0 refers to the methylation level of DNA CpG site of NNMT gene in the cell with normal or low methylation level of DNA CpG site of NNMT gene.

In another preferred embodiment, the cell with normal or low methylation level of DNA CpG site of NNMT gene comprises the cell that is not sensitive to the compound of formula I, or an optical isomer thereof, or a racemate thereof, or a solvate thereof, or a pharmaceutically acceptable salt thereof, or a deuterated compound thereof.

In another preferred embodiment, the high methylation level of DNA CpG site of NNMT gene means the methylation level (M %) of DNA CpG site of NNMT gene in a cell (e.g. tumor cell) is ≥3% and ≤M2%, wherein M2 is any positive integer from 3 to 100.

In another preferred embodiment, M2 is 5, 10, 15, 20, 25, 30, 35, 40, 45, 50, 55, 60, 65, 70, 80, 85, 90, 95 or 100.

In another preferred embodiment, the methylation level of DNA CpG site of NNMT gene refers to the ratio of the number of methylated CpG nucleotides to the number of all nucleotides in the NNMT gene.

In another preferred embodiment, the methylation level of DNA CpG site of NNMT gene refers to the ratio of the number of methylated DNA CpG nucleotides to the number of all CpG nucleotides in the NNMT gene.

In another preferred embodiment, the methylation level of DNA CpG site of NNMT gene refers to the ratio of the number of methylated DNA CpG sites to the number of all DNA CpG sites in the NNMT gene.

In another preferred embodiment, the methylation level of DNA CpG site of NNMT gene refers to the ratio of the number of methylated DNA CpG nucleotides to the number of all DNA CpG nucleotides in the NNMT gene.

In another preferred embodiment, the methylation level of DNA CpG site of NNMT gene comprises the methylation level of DNA CpG site in promoter region of NNMT gene.

In another preferred embodiment, the nucleotide sequence of the promoter region of NNMT gene is as shown in SEQ ID NO: 1.

In another preferred embodiment, the methylation level of DNA CpG site of NNMT gene comprises the methylation level of DNA CpG sites from 1050 bp before the transcription start site to 499 bp after the transcription start site in NNMT gene.

In another preferred embodiment, the sites from 1050 bp before the transcription start site to 499 bp after the transcription start site in NNMT gene is sites 951-2500 of nucleotide sequence as shown in SEQ ID NO: 1.

In another preferred embodiment, the methylation level of DNA CpG site of NNMT gene comprises the methylation level of the DNA CpG sites from 1050 bp to 193 bp before the transcription start site in NNMT gene.

In another preferred embodiment, the sites from 1050 bp to 193 bp before the transcription start site in NNMT gene is sites 951-1808 of nucleotide sequence as shown in SEQ ID NO: 1.

In another preferred embodiment, the methylation level of DNA CpG site of NNMT gene comprises the methylation level of DNA CpG sites from 840 bp to 469 bp before the transcription start site in NNMT gene.

In another preferred embodiment, the sites from 840 bp to 469 bp before the transcription start site in NNMT gene is sites 1161-1532 of nucleotide sequence as shown in SEQ ID NO: 1.

In another preferred embodiment, the methylation level of DNA CpG site of NNMT gene comprises the methylation level of DNA CpG site between any two sites (including the two sites itself) selected from group consisting of site 114165695, site 114165730, site 114165769, site 114165804, site 114165938, site 114166050 and site 114166066 on human chromosome 11.

In another preferred embodiment, the methylation level of DNA CpG site of NNMT gene comprises the methylation level of sites selected from group consisting of site 114165695 on human chromosome 11, site 114165730 on human chromosome 11, site 114165769 on human chromosome 11, site 114165804 on human chromosome 11, site 114165938 on human chromosome 11, site 114166050 on human chromosome 11, site 114166066 on human chromosome 11, and combinations thereof.

In another preferred embodiment, the methylation level of DNA CpG site of NNMT gene comprises the methylation level of DNA CpG site between any two sites (including the two sites itself) selected from group consisting of site 1161, site 1196, site 1235, site 1270, site 1404, site 1516 and site 1532 in nucleotide sequence of SEQ ID NO: 1.

In another preferred embodiment, the methylation level of DNA CpG site of NNMT gene comprises the methylation level of sites selected from group consisting of site 1161 in SEQ ID NO: 1, site 1196 in SEQ ID NO: 1, site 1235 in SEQ ID NO: 1, site 1270 in SEQ ID NO: 1, site 1404 in SEQ ID NO: 1, site 1516 in SEQ ID NO: 1, site 1532 in SEQ ID NO: 1, and combinations thereof.

In another preferred embodiment, the NNMT gene inhibitor is administered to achieve low or no expression of the NNMT gene in tumor.

In another preferred embodiment, the DNA methylase promoter is administered to achieve high expression of DNA methylase in tumor.

In another preferred embodiment, the DNMT1 promoter is administered to achieve high expression of DNMT1 in tumor.

In another preferred embodiment, the DNMT3a promoter is administered to achieve high expression of DNMT3a in tumor.

In another preferred embodiment, the DNMT3b promoter is administered to achieve high expression of DNMT3b in tumor.

In another preferred embodiment, the UHRF1 promoter is administered to achieve high expression of UHRF1 in tumor.

In another preferred embodiment, the methylation promoter of nucleotide site of NNMT gene is administered to achieve high methylation level of nucleotide site of NNMT gene in tumor.

In another preferred embodiment, the methylation promoter of DNA CpG site of NNMT gene is administered to achieve high methylation level of DNA CpG site of NNMT gene in tumor.

In another preferred embodiment, the inhibitor comprises specific inhibitor.

In another preferred embodiment, the promoter comprises specific promoter.

In another preferred embodiment, the NNMT gene inhibitor comprises inhibitor that can achieve low or no expression of the NNMT gene in tumor.

In another preferred embodiment, the DNA methylase promoter comprises promoter that can achieve high expression of DNA methylase in tumor.

In another preferred embodiment, the DNMT1 promoter comprises promoter that can achieve high expression of DNMT1 in tumor.

In another preferred embodiment, the DNMT3a promoter comprises promoter that can achieve high expression of DNMT3a in tumor.

In another preferred embodiment, the DNMT3b promoter comprises promoter that can achieve high expression of DNMT3b in tumor.

In another preferred embodiment, the UHRF1 promoter comprises promoter that can achieve high expression of UHRF1 in tumor.

In another preferred embodiment, the methylation promoter of nucleotide site of NNMT gene comprises promoter that can achieve high methylation level of nucleotide site of NNMT gene in tumor.

In another preferred embodiment, the methylation promoter of DNA CpG site of NNMT gene comprises promoter that can achieve high methylation promoter of DNA CpG site of NNMT gene in tumor.

In another preferred embodiment, the tumor is selected from the group consisting of lung cancer, renal carcinoma, breast cancer, colon cancer, lymphoma, leukemia, pancreatic cancer, brain tumor, liver cancer, prostate cancer, and combinations thereof.

In another preferred embodiment, the lung cancer is selected from the group consisting of non-small cell lung cancer, small cell lung cancer, and combinations thereof.

In another preferred embodiment, the lung cancer cell comprises NCI-H82 cell.

In another preferred embodiment, the colon cancer comprises colon adenocarcinoma.

In another preferred embodiment, the colon cancer cell comprises SW48 cell.

In another preferred embodiment, the breast cancer cell comprises MDA-MB-453 cell.

In another preferred embodiment, the breast cancer comprises triple negative breast cancer.

In another preferred embodiment, the lymphoma is selected from the group consisting of B-cell lymphoma, skin T-cell lymphoma, and combinations thereof.

In another preferred embodiment, the lymphoma comprises diffuse large B-cell lymphoma.

In another preferred embodiment, the brain tumor is selected from the group consisting of brain glioblastoma, neuroglioma, brain medulloblastoma, brain neuroblastoma, and combination thereof.

In another preferred embodiment, the brain medulloblastoma comprises cerebellar medulloblastoma.

In another preferred embodiment, the brain glioblastoma comprises glioblastoma multiforme.

In another preferred embodiment, the brain tumor comprises glioblastoma.

In another preferred embodiment, the brain tumor cell comprises one or more of GB-1 cell, Daoy cell, and SF126 cell.

In another preferred embodiment, the renal carcinoma is selected from the group consisting of clear cell renal cell adenocarcinoma, renal carcinoma Wilms, and combination thereof.

In another preferred embodiment, the renal carcinoma comprises clear cell renal cell adenocarcinoma.

In another preferred embodiment, the renal carcinoma comprises renal carcinoma Wilms.

In another preferred embodiment, the renal carcinoma cell comprises renal carcinoma Wilms cell.

In another preferred embodiment, the renal carcinoma cell comprises one or more of G-401 cell and 786-O cell.

In another preferred embodiment, the pancreatic cancer cell comprises CFPAC-1 cell.

In another preferred embodiment, the leukemia is selected from the group consisting of T-lymphocyte leukemia, myeloid leukemia, and combinations thereof.

In another preferred embodiment, the T-lymphocytic leukemia comprises acute T-lymphocytic leukemia.

In another preferred embodiment, the myeloid leukemia comprises type M4 of acute myeloid leukemia.

In another preferred embodiment, the myeloid leukemia comprises FAB type M4 of acute myeloid leukemia.

In another preferred embodiment, the expression comprises protein expression and/or mRNA expression.

In another preferred embodiment, the composition or preparation is a pharmaceutical composition or pharmaceutical preparation.

In another preferred embodiment, the composition or preparation further comprises a pharmaceutically acceptable carrier.

In another preferred embodiment, the expression is mRNA expression or protein expression.

In another preferred embodiment, the dosage form of the composition or preparation is a solid preparation, liquid preparation or semi-solid preparation.

In another preferred embodiment, the dosage form of the composition or preparation is oral preparation, external preparation or injection preparation.

In the fourth aspect of the present invention, it provides a marker for determining whether the compound of formula I, or an optical isomer thereof, or a racemate thereof, or a solvate thereof, or a pharmaceutically acceptable salt thereof, or a deuterated compound thereof according to the first aspect of the present invention is suitable for use in the prevention and/or treatment of patient tumor, the marker comprises mitochondria permeability transition pore, peptidyl-prolyl cis-trans isomerase F, NNMT gene, DNA methylase, UHRF1, the methylation of nucleotide site of NNMT gene, and/or the methylation of DNA CpG site of NNMT gene.

In another preferred embodiment, the marker comprises the expression level or activity of mitochondria permeability transition pore, the expression level or activity of peptidyl-prolyl cis-trans isomerase F, the expression level of NNMT gene, the expression level of DNA methylase, the expression level of UHRF1, the methylation level of nucleotide site of NNMT gene, and/or the methylation level of DNA CpG site of NNMT gene.

In another preferred embodiment, the DNA methylase is selected from the group consisting of DNMT1, DNMT3a, DNMT3b, and combinations thereof.

In another preferred embodiment, the mitochondria permeability transition pore, peptidyl-prolyl cis-trans isomerase F, NNMT gene, DNA methylase, UHRF1, the methylation of nucleotide site of NNMT gene, and/or the methylation of DNA CpG site of NNMT gene comprises the mitochondria permeability transition pore, peptidyl-prolyl cis-trans isomerase F, NNMT gene, DNA methylase, UHRF1, the methylation of nucleotide site of NNMT gene, and/or the methylation of DNA CpG site of NNMT gene in the tumor cell.

In another preferred embodiment, the compound of formula I, or an optical isomer thereof, or a racemate thereof, or a solvate thereof, or a pharmaceutically acceptable salt thereof, or a deuterated compound thereof according to the first aspect of the present invention is suitable for use in the prevention and/or treatment of patient tumor with low expression, no expression, low activity or no activity of mitochondria permeability transition pore, low expression, no expression, low activity or no activity of peptidyl-prolyl cis-trans isomerase F, low or no expression of NNMT gene, high expression of DNA methylase, high expression of UHRF1, high methylation level of nucleotide site of NNMT gene, and/or high methylation level of DNA CpG site of NNMT gene.

In another preferred embodiment, the compound of formula I, or an optical isomer thereof, or a racemate thereof, or a solvate thereof, or a pharmaceutically acceptable salt thereof, or a deuterated compound thereof according to the first aspect of the present invention is not suitable for use in the prevention and/or treatment of patient tumor with high expression or high activity of mitochondria permeability transition pore, high expression or high activity of peptidyl-prolyl cis-trans isomerase F, high expression of NNMT gene, low expression of DNA methylase, low expression of UHRF1, low methylation level of nucleotide site of NNMT gene, and/or low methylation level of DNA CpG site of NNMT gene.

In another preferred embodiment, “the compound of formula I, or an optical isomer thereof, or a racemate thereof, or a solvate thereof, or a pharmaceutically acceptable salt thereof, or a deuterated compound thereof according to the first aspect of the present invention is suitable for use in the prevention and/or treatment of patient tumor” comprises “the patient tumor is sensitive to the compound of formula I, or an optical isomer thereof, or a racemate thereof, or a solvate thereof, or a pharmaceutically acceptable salt thereof, or a deuterated compound thereof according to the first aspect of the present invention”.

In another preferred embodiment, “the compound of formula I, or an optical isomer thereof, or a racemate thereof, or a solvate thereof, or a pharmaceutically acceptable salt thereof, or a deuterated compound thereof according to the first aspect of the present invention is not suitable for use in the prevention and/or treatment of patient tumor” comprises “the patient tumor is not sensitive to the compound of formula I, or an optical isomer thereof, or a racemate thereof, or a solvate thereof, or a pharmaceutically acceptable salt thereof, or a deuterated compound thereof according to the first aspect of the present invention”.

In another preferred embodiment, the high expression or high activity of mitochondria permeability transition pore means the ratio (H1/H0) of the expression level or activity level H1 of mitochondria permeability transition pore in a cell (e.g. tumor cell) to the expression level or activity level H0 of mitochondria permeability transition pore in the same type of cell or a normal cell (e.g. para-tumor tissue cell) is >1.0, preferably ≥1.2, more preferably ≥1.5, more preferably ≥2, more preferably ≥3, more preferably ≥5, more preferably ≥8, more preferably ≥10, more preferably ≥15, more preferably ≥20, more preferably ≥30, more preferably ≥50, such as 2-50.

In another preferred embodiment, the high expression or high activity of peptidyl-prolyl cis-trans isomerase F means the ratio (P1/P0) of the expression level or activity level P1 of peptidyl-prolyl cis-trans isomerase F in a cell (e.g. tumor cell) to the expression level or activity level P0 of peptidyl-prolyl cis-trans isomerase F in the same type of cell or a normal cell (e.g. para-tumor tissue cell) is ≥1.0, preferably ≥1.2, more preferably ≥1.5, more preferably ≥2, more preferably ≥3, more preferably ≥5, more preferably ≥8, more preferably ≥10, more preferably ≥15, more preferably ≥20, more preferably ≥30, more preferably ≥50, such as 2-50.

In another preferred embodiment, the high expression of NNMT gene means the ratio (E1/E0) of the expression E1 of NNMT gene in a cell (e.g. tumor cell) to the expression E0 of NNMT gene in the same type of cell or a normal cell is >1.0, preferably ≥1.2, more preferably ≥1.5, more preferably ≥2, more preferably ≥3, more preferably ≥5, more preferably ≥8, more preferably ≥10, more preferably ≥15, more preferably ≥20, more preferably ≥30, more preferably ≥50, such as 2-50.

In another preferred embodiment, the tumor with low expression of DNA methylase means the ratio (A1/A0) of the expression level A1 of DNA methylase in the tumor cell to the expression level A0 of DNA methylase in the same type of cell or a normal cell is <1.0, preferably ≤0.7, more preferably ≤0.6, more preferably ≤0.5, more preferably ≤0.4, more preferably ≤0.3, more preferably ≤0.2, more preferably ≤0.1, more preferably ≤0.05, more preferably ≤0.01, more preferably ≤0.005, more preferably ≤0.001, more preferably ≤0.0001, more preferably ≤0.00001, more preferably ≤0.000001, more preferably ≤0.0000001.

In another preferred embodiment, the tumor with low expression of UHRF1 means the ratio (F1/F0) of the expression level F1 of UHRF1 in the tumor cell to the expression level F0 of UHRF1 in the same type of cell or a normal cell is <1.0, preferably ≤0.7, more preferably ≤0.6, more preferably ≤0.5, more preferably ≤0.4, more preferably ≤0.3, more preferably ≤0.2, more preferably ≤0.1, more preferably ≤0.05, more preferably ≤0.01, more preferably ≤0.005, more preferably ≤0.001, more preferably ≤0.0001, more preferably ≤0.00001, more preferably ≤0.000001, more preferably ≤0.0000001.

In another preferred embodiment, the low methylation level of nucleotide site of NNMT gene means the ratio (L1/L0) of the methylation level L1 of nucleotide site of NNMT gene in a cell (e.g. tumor cell) to the methylation level L0 of nucleotide site of NNMT gene in the same type of cell or a normal cell is <1.0, preferably ≤0.7, more preferably ≤0.6, more preferably ≤0.5, more preferably ≤0.4, more preferably ≤0.3, more preferably ≤0.2, more preferably ≤0.1, more preferably ≤0.05, more preferably ≤0.01, more preferably ≤0.005, more preferably ≤0.001, more preferably ≤0.0001, more preferably ≤0.00001, more preferably ≤0.000001, more preferably ≤0.0000001.

In another preferred embodiment, the low methylation level of DNA CpG site of NNMT gene means the ratio (G1/G0) of the methylation level G1 of DNA CpG site of NNMT gene in a cell (e.g. tumor cell) to the methylation level G0 of DNA CpG site of NNMT gene in the same type of cell or a normal cell is <1.0, preferably ≤0.7, more preferably ≤0.6, more preferably ≤0.5, more preferably ≤0.4, more preferably ≤0.3, more preferably ≤0.2, more preferably ≤0.1, more preferably ≤0.05, more preferably ≤0.01, more preferably ≤0.005, more preferably ≤0.001, more preferably ≤0.0001, more preferably ≤0.00001, more preferably ≤0.000001, more preferably ≤0.0000001.

In the fifth aspect of the present invention, it provides a detection kit, which comprises: (i) a detection reagent for detecting the expression level or activity of mitochondria permeability transition pore, the expression level or activity of peptidyl-prolyl cis-trans isomerase F, the expression level of NNMT gene, the expression level of DNA methylase, the expression level of UHRF1, the methylation level of nucleotide site of NNMT gene, and/or the methylation level of DNA CpG site of NNMT gene.

In another preferred embodiment, the test sample of the detection kit comprises tumor.

In another preferred embodiment, the expression of NNMT gene is the expression of mRNA and/or protein.

In the sixth aspect of the present invention, it provides a use of the detection kit according to the fifth aspect of the present invention in the preparation of concomitant diagnose kit for determining whether the compound of formula I, or an optical isomer thereof, or a racemate thereof, or a solvate thereof, or a pharmaceutically acceptable salt thereof, or a deuterated compound thereof according to the first aspect of the present invention is suitable for use in the prevention and/or treatment of patient tumor.

In another preferred embodiment, the concomitant diagnose kit further comprises instruction or label.

In another preferred embodiment, the instruction or label records that the compound of formula I, or an optical isomer thereof, or a racemate thereof, or a solvate thereof, or a pharmaceutically acceptable salt thereof, or a deuterated compound thereof according to the first aspect of the present invention is suitable for use in the prevention and/or treatment of patient tumor with low expression, no expression, low activity or no activity of mitochondria permeability transition pore, low expression, no expression, low activity or no activity of peptidyl-prolyl cis-trans isomerase F, low or no expression of NNMT gene, high expression of DNA methylase, high expression of UHRF1, high methylation level of nucleotide site of NNMT gene, and/or high methylation level of DNA CpG site of NNMT gene.

In another preferred embodiment, the instruction or label records that the compound of formula I, or an optical isomer thereof, or a racemate thereof, or a solvate thereof, or a pharmaceutically acceptable salt thereof, or a deuterated compound thereof according to the first aspect of the present invention is not suitable for use in the prevention and/or treatment of patient tumor with high expression or high activity of mitochondria permeability transition pore, high expression or high activity of peptidyl-prolyl cis-trans isomerase F, high expression of NNMT gene, low expression of DNA methylase, low expression of UHRF1, low methylation level of nucleotide site of NNMT gene, and/or low methylation level of DNA CpG site of NNMT gene.

In another preferred embodiment, “the compound of formula I, or an optical isomer thereof, or a racemate thereof, or a solvate thereof, or a pharmaceutically acceptable salt thereof, or a deuterated compound thereof according to the first aspect of the present invention is suitable for use in the prevention and/or treatment of patient tumor” is as described in the fourth aspect of the present invention.

In another preferred embodiment, “the compound of formula I, or an optical isomer thereof, or a racemate thereof, or a solvate thereof, or a pharmaceutically acceptable salt thereof, or a deuterated compound thereof according to the first aspect of the present invention is not suitable for use in the prevention and/or treatment of patient tumor” is as described in the fourth aspect of the present invention.

In the seventh aspect of the present invention, it provides a medicine kit, which comprises:
(i) a detection reagent for detecting the expression level or activity of mitochondria permeability transition pore, the expression level or activity of peptidyl-prolyl cis-trans isomerase F, the expression level of NNMT gene, the expression level of DNA methylase, the expression level of UHRF1, the methylation level of nucleotide site of NNMT gene, and/or the methylation level of DNA CpG site of NNMT gene; and
(ii) the compound of formula I, or an optical isomer thereof, or a racemate thereof, or a solvate thereof, or a pharmaceutically acceptable salt thereof, or a deuterated compound thereof according to the first aspect of the present invention.

In another preferred embodiment, the detection sample comprises tumor.

In another preferred embodiment, the medicine kit further comprises instruction or label.

In another preferred embodiment, the instruction or label records that the compound of formula I, or an optical isomer thereof, or a racemate thereof, or a solvate thereof, or a pharmaceutically acceptable salt thereof, or a deuterated compound thereof according to the first aspect of the present invention is suitable for use in the prevention and/or treatment of patient tumor with low expression, no expression, low activity or no activity of mitochondria permeability transition pore, low expression, no expression, low activity or no activity of peptidyl-prolyl cis-trans isomerase F, low or no expression of NNMT gene, high expression of DNA methylase, high expression of UHRF1, high methylation level of nucleotide site of NNMT gene, and/or high methylation level of DNA CpG site of NNMT gene.

In another preferred embodiment, the instruction or label records that the compound of formula I, or an optical isomer thereof, or a racemate thereof, or a solvate thereof, or a pharmaceutically acceptable salt thereof, or a deuterated compound thereof according to the first aspect of the present invention is not suitable for use in the prevention and/or treatment of patient tumor with high expression or high activity of mitochondria permeability transition pore, high expression or high activity of peptidyl-prolyl cis-trans isomerase F, high expression of NNMT gene, low expression of DNA methylase, low expression of UHRF1, low methylation level of nucleotide site of NNMT gene, and/or low methylation level of DNA CpG site of NNMT gene.

In the eighth aspect of the present invention, it provides a method for preventing and/or treating tumor, which comprises administering the compound of formula I, or an optical isomer thereof, or a racemate thereof, or a solvate thereof, or a pharmaceutically acceptable salt thereof, or a deuterated compound thereof according to the first aspect of the present invention to a subject in need, thereby preventing and/or treating tumor.

In another preferred embodiment, the tumor is as described in the third aspect of the invention.

In another preferred embodiment, the subject is human and non-human mammals (rodent, rabbit, monkey, livestock, dog, cat, etc.).

In another preferred embodiment, the method comprises:
firstly achieving low expression, no expression, low activity or no activity of mitochondria permeability transition pore, low expression, no expression, low activity or no activity of peptidyl-prolyl cis-trans isomerase F, low or no expression of NNMT gene, high expression of DNA methylase, high expression of UHRF1, high methylation level of nucleotide site of NNMT gene, and/or high methylation level of DNA CpG site of NNMT gene in the subject tumor, then administering the compound of formula I, or an optical isomer thereof, or a racemate thereof, or a solvate thereof, or a pharmaceutically acceptable salt thereof, or a deuterated compound thereof to prevent and/or treat tumor.

In another preferred embodiment, the method comprises:
firstly administering mitochondria permeability transition pore inhibitor, peptidyl-prolyl cis-trans isomerase F inhibitor, NNMT gene inhibitor, DNA methylase promoter, UHRF1 promoter, methylation promoter of nucleotide site of NNMT gene, and/or methylation promoter of DNA CpG site of NNMT gene to achieve low expression, no expression, low activity or no activity of mitochondria permeability transition pore, low expression, no expression, low activity or no activity of peptidyl-prolyl cis-trans isomerase F, low or no expression of NNMT gene, high expression of DNA methylase, high expression of UHRF1, high methylation level of nucleotide site of NNMT gene, and/or high methylation level of DNA CpG site of NNMT gene in the subject tumor, then administering the compound of formula I, or an optical isomer thereof, or a racemate thereof, or a solvate thereof, or a pharmaceutically acceptable salt thereof, or a deuterated compound thereof to prevent and/or treat tumor.

In another preferred embodiment, the mitochondria permeability transition pore inhibitor, peptidyl-prolyl cis-trans isomerase F inhibitor, NNMT gene inhibitor, DNA methylase promoter, UHRF1 promoter, methylation promoter of nucleotide site of NNMT gene, and/or methylation promoter of DNA CpG site of NNMT gene is as described in the third aspect of the invention.

In the ninth aspect of the present invention, it provides a device or system, the device or system comprises:
(i) a detection module, the detection module is used to detect the expression level or activity of mitochondria permeability transition pore, the expression level or activity of peptidyl-prolyl cis-trans isomerase F, the expression level of NNMT gene, the expression level of DNA methylase, the expression level of UHRF1, the methylation level of nucleotide site of NNMT gene, and/or the methylation level of DNA CpG site of NNMT gene;
(ii) an output module, the output module comprises the information as follows:
the compound of formula I, or an optical isomer thereof, or a racemate thereof, or a solvate thereof, or a pharmaceutically acceptable salt thereof, or a deuterated compound thereof according to the first aspect of the present invention is suitable for use in the prevention and/or treatment of patient tumor with low expression, no expression, low activity or no activity of mitochondria permeability transition pore, low expression, no expression, low activity or no activity of peptidyl-prolyl cis-trans isomerase F, low or no expression of NNMT gene, high expression of DNA methylase, high expression of UHRF1, high methylation level of nucleotide site of NNMT gene, and/or high methylation level of DNA CpG site of NNMT gene; and/or
the compound of formula I, or an optical isomer thereof, or a racemate thereof, or a solvate thereof, or a pharmaceutically acceptable salt thereof, or a deuterated compound thereof according to the first aspect of the present invention is not suitable for use in the prevention and/or treatment of patient tumor with high expression or high activity of mitochondria permeability transition pore, high expression or high activity of peptidyl-prolyl cis-trans isomerase F, high expression of NNMT gene, low expression of DNA methylase, low expression of UHRF1, low methylation level of nucleotide site of NNMT gene, and/or low methylation level of DNA CpG site of NNMT gene.

In another preferred embodiment, the detection sample comprises tumor.

In another preferred embodiment, the device comprises a gene detector or protein detector.

In another preferred embodiment, the device or system further comprises sample injection module.

In another preferred embodiment, the injection module is used to inject tumor cell extract.

In another preferred embodiment, the device or system further comprises data processing module.

In another preferred embodiment, the expression level or activity of mitochondria permeability transition pore, the expression level or activity of peptidyl-prolyl cis-trans isomerase F, the expression level of NNMT gene, the expression level of DNA methylase, the expression level of UHRF1, the methylation level of nucleotide site of NNMT gene, and/or the methylation level of DNA CpG site of NNMT gene can be obtained by the procession of the data processing module.

In the tenth aspect of the present invention, it provides a use of mitochondria permeability transition pore inhibitor, peptidyl-prolyl cis-trans isomerase F inhibitor, NNMT gene inhibitor, DNA methylase promoter, UHRF1 promoter, methylation promoter of nucleotide site of NNMT gene, and/or methylation promoter of DNA CpG site of NNMT gene in the preparation of a composition or a preparation for enhancing the anti-tumor effect of anti-tumor drug.

In another preferred embodiment, the mitochondria permeability transition pore inhibitor comprises inhibitor that can achieve low expression, no expression, low activity or no activity of mitochondria permeability transition pore in tumor.

In another preferred embodiment, the peptidyl-prolyl cis-trans isomerase F inhibitor comprises inhibitor that can achieve low expression, no expression, low activity or no activity of peptidyl-prolyl cis-trans isomerase F.

In another preferred embodiment, the NNMT gene inhibitor comprises inhibitor that can achieve low or no expression of the NNMT gene in tumor.

In another preferred embodiment, the DNA methylase is selected from the group consisting of DNMT1, DNMT3a, DNMT3b, and combinations thereof.

In another preferred embodiment, the DNA methylase promoter comprises promoter that can achieve high expression of DNA methylase in tumor.

In another preferred embodiment, the DNA methylase promoter comprises DNMT1 promoter.

In another preferred embodiment, the DNMT1 promoter comprises promoter that can achieve high expression of DNMT1 in tumor.

In another preferred embodiment, the DNA methylase promoter comprises DNMT3a promoter.

In another preferred embodiment, the DNMT3a promoter comprises promoter that can achieve high expression of DNMT3a in tumor.

In another preferred embodiment, the DNA methylase promoter comprises DNMT3b promoter.

In another preferred embodiment, the DNMT3b promoter comprises promoter that can achieve high expression of DNMT3b in tumor.

In another preferred embodiment, the UHRF1 promoter comprises promoter that can achieve high expression of UHRF1 in tumor.

In another preferred embodiment, the methylation promoter of nucleotide site of NNMT gene comprises promoter that can achieve high methylation level of nucleotide site of NNMT gene in tumor.

In another preferred embodiment, the methylation promoter of DNA CpG site of NNMT gene comprises promoter that can achieve high methylation level of nucleotide site of NNMT gene in tumor.

In another preferred embodiment, the inhibitor comprises specific inhibitor.

In another preferred embodiment, the promoter comprises specific promoter.

In another preferred embodiment, the anti-tumor drug comprises the compound of formula I, or an optical isomer thereof, or a racemate thereof, or a solvate thereof, or a pharmaceutically acceptable salt thereof, or a deuterated compound thereof according to the first aspect of the present invention.

In another preferred embodiment, the tumor is as described in the third aspect of the invention.

In another preferred embodiment, the mitochondria permeability transition pore inhibitor is selected from the group consisting of Cyclosporin A, CyP-D protein inhibitor, peroxide scavenger, and combinations thereof.

In another preferred embodiment, the peptidyl-prolyl cis-trans isomerase F inhibitor comprises shRNA.

In another preferred embodiment, the nucleotide sequence of shRNA is

GTTCTTCATCTGCACCATAAA.

In another preferred embodiment, the dosage form of the composition or preparation is a solid preparation, liquid preparation or semi-solid preparation.

In another preferred embodiment, the dosage form of the composition or preparation is oral preparation, external preparation or injection preparation.

In another preferred embodiment, the dosage form of the composition or preparation is tablet, injection, infusion, paste, gel, solution, microsphere or film.

In the eleventh aspect of the present invention, it provides an active ingredient combination, the active ingredient combination comprises:
(1) a first active ingredient, the first active ingredient comprises anti-tumor drug; and
(2) a second active ingredient, the second active ingredient comprises mitochondria permeability transition pore inhibitor, peptidyl-prolyl cis-trans isomerase F inhibitor, NNMT gene inhibitor, DNA methylase promoter, UHRF1 promoter, methylation promoter of nucleotide site of NNMT gene, and/or methylation promoter of DNA CpG site of NNMT gene.

In another preferred embodiment, the anti-tumor drug comprises the compound of formula I, or an optical isomer thereof, or a racemate thereof, or a solvate thereof, or a pharmaceutically acceptable salt thereof, or a deuterated compound thereof according to the first aspect of the present invention.

In another preferred embodiment, the mitochondria permeability transition pore inhibitor, peptidyl-prolyl cis-trans isomerase F inhibitor, NNMT gene inhibitor, DNA methylase promoter, UHRF1 promoter, methylation promoter of nucleotide site of NNMT gene, and/or methylation promoter of DNA CpG site of NNMT gene is as described in the tenth aspect of the invention.

In another preferred embodiment, the molar ratio of the first active ingredient to the second active ingredient is 0.01-600:1, preferably 0.05-500:1, more preferably 0.1-400:1, more preferably 0.2-200:1, more preferably 0.5-100:1, more preferably 0.5-80:1, most preferably 1-50:1.

In another preferred embodiment, at least one active ingredient is independent in the active ingredient combination.

In another preferred embodiment, the first active ingredient and the second active ingredient are independent of each other in the active ingredient combination.

In the twelfth aspect of the present invention, it provides a composition, the composition comprises:
(1) a first active ingredient, the first active ingredient comprises anti-tumor drug; and
(2) a second active ingredient, the second active ingredient comprises mitochondria permeability transition pore inhibitor, peptidyl-prolyl cis-trans isomerase F inhibitor, NNMT gene inhibitor, DNA methylase promoter, UHRF1 promoter, methylation promoter of nucleotide site of NNMT gene, and/or methylation promoter of DNA CpG site of NNMT gene.

In another preferred embodiment, the anti-tumor drug comprises the compound of formula I, or an optical isomer thereof, or a racemate thereof, or a solvate thereof, or a pharmaceutically acceptable salt thereof, or a deuterated compound thereof according to the first aspect of the present invention.

In another preferred embodiment, the mitochondria permeability transition pore inhibitor, peptidyl-prolyl cis-trans isomerase F inhibitor, NNMT gene inhibitor, DNA methylase promoter, UHRF1 promoter, methylation promoter of nucleotide site of NNMT gene, and/or methylation promoter of DNA CpG site of NNMT gene is as described in the tenth aspect of the invention.

In another preferred embodiment, the composition is a pharmaceutical composition.

In another preferred embodiment, the pharmaceutical composition further comprises a pharmaceutically acceptable carrier.

In another preferred embodiment, the dosage form of the composition or preparation is a solid preparation, liquid preparation or semi-solid preparation.

In another preferred embodiment, the dosage form of the composition or preparation is oral preparation, external preparation or injection preparation.

In another preferred embodiment, the content of the first active ingredient is 0.01-99.99 wt %, preferably 0.1-99.9 wt %, more preferably 1-99 wt %, more preferably 10-99 wt %, most preferably 20-99 wt %, based on the total weight of the active ingredient in the composition.

In another preferred embodiment, the content of the second active ingredient is 0.01-99.99 wt %, preferably 0.1-99.9 wt %, more preferably 1-99 wt %, more preferably 10-99 wt %, most preferably 20-99 wt %, based on the total weight of the active ingredient in the composition. In the thirteenth aspect of the present invention, it provides a medicine kit, the medicine kit comprises:
(A) a first preparation comprising a first active ingredient, the first active ingredient comprises anti-tumor drug; and
(B) a second preparation comprising a second active ingredient, the second active ingredient comprises mitochondria permeability transition pore inhibitor, peptidyl-prolyl cis-trans isomerase F inhibitor, NNMT gene inhibitor, DNA methylase promoter, UHRF1 promoter, methylation promoter of nucleotide site of NNMT gene, and/or methylation promoter of DNA CpG site of NNMT gene.

In another preferred embodiment, the anti-tumor drug comprises the compound of formula I, or an optical isomer thereof, or a racemate thereof, or a solvate thereof, or a pharmaceutically acceptable salt thereof, or a deuterated compound thereof according to the first aspect of the present invention.

In another preferred embodiment, the mitochondria permeability transition pore inhibitor, peptidyl-prolyl cis-trans isomerase F inhibitor, NNMT gene inhibitor, DNA methylase promoter, UHRF1 promoter, methylation promoter of nucleotide site of NNMT gene, and/or methylation promoter of DNA CpG site of NNMT gene is as described in the tenth aspect of the invention.

In another preferred embodiment, the medicine kit further comprises a user manual.

In another preferred embodiment, the first preparation and the second preparation are independent preparation of each other.

In another preferred embodiment, the first preparation and the second preparation are combined preparation.

In another preferred embodiment, the user manual records that the first preparation and the second preparation are used together to enhance anti-tumor activity of anti-tumor drug.

In another preferred embodiment, the method for using together means the second preparation comprising a second active ingredient is administered firstly, then the first preparation comprising a first active ingredient is administered.

In the fourteenth aspect of the present invention, it provides a method for inhibiting tumor cell, which comprises contacting the tumor cell with the compound of formula I, or an optical isomer thereof, or a racemate thereof, or a solvate thereof, or a pharmaceutically acceptable salt thereof, or a deuterated compound thereof according to the first aspect of the present invention, thereby inhibiting tumor cell.

In another preferred embodiment, the method is vitro method.

In another preferred embodiment, the method is non-therapeutic and non-diagnostic method.

In another preferred embodiment, the contact is performed in vitro culture.

In another preferred embodiment, the tumor is as described in the third aspect of the invention.

In another preferred embodiment, the method comprises:
firstly achieving low expression, no expression, low activity or no activity of mitochondria permeability transition pore, low expression, no expression, low activity or no activity of peptidyl-prolyl cis-trans isomerase F, low or no expression of NNMT gene, high expression of DNA methylase, high expression of UHRF1, high methylation level of nucleotide site of NNMT gene, and/or high methylation level of DNA CpG site of NNMT gene in the tumor cell, then contacting the tumor cell with the compound of formula I, or an optical isomer thereof, or a racemate thereof, or a solvate thereof, or a pharmaceutically acceptable salt thereof, or a deuterated compound thereof according to the first aspect of the present invention, thereby inhibiting tumor cell.

In another preferred embodiment, the method comprises:
firstly administering mitochondria permeability transition pore inhibitor, peptidyl-prolyl cis-trans isomerase F inhibitor, NNMT gene inhibitor, DNA methylase promoter, UHRF1 promoter, methylation promoter of nucleotide site of NNMT gene, and/or methylation promoter of DNA CpG site of NNMT gene to achieve low expression, no expression, low activity or no activity of mitochondria permeability transition pore, low expression, no expression, low activity or no activity of peptidyl-prolyl cis-trans isomerase F, low or no expression of NNMT gene, high expression of DNA methylase, high expression of UHRF1, high methylation level of nucleotide site of NNMT gene, and/or high methylation level of DNA CpG site of NNMT gene in the tumor cell, then contacting the tumor cell with the compound of formula I, or an optical isomer thereof, or a racemate thereof, or a solvate thereof, or a pharmaceutically acceptable salt thereof, or a deuterated compound thereof according to the first aspect of the present invention, thereby inhibiting tumor cell.

In another preferred embodiment, the mitochondria permeability transition pore inhibitor, peptidyl-prolyl cis-trans isomerase F inhibitor, NNMT gene inhibitor, DNA methylase promoter, UHRF1 promoter, methylation promoter of nucleotide site of NNMT gene, and/or methylation promoter of DNA CpG site of NNMT gene is as described in the tenth aspect of the invention.

In the fifteenth aspect of the present invention, it provides a use of medicine kit according to the seventh aspect of the present invention in the preparation of medicine box for preventing and/or treating tumor.

In another preferred embodiment, the medicine box further comprises instruction or label.

In another preferred embodiment, the instruction or label records that the compound of formula I, or an optical isomer thereof, or a racemate thereof, or a solvate thereof, or a pharmaceutically acceptable salt thereof, or a deuterated compound thereof according to the first aspect of the present invention is suitable for use in the prevention and/or treatment of patient tumor with low expression, no expression, low activity or no activity of mitochondria permeability transition pore, low expression, no expression, low activity or no activity of peptidyl-prolyl cis-trans isomerase F, low or no expression of NNMT gene, high expression of DNA methylase, high expression of UHRF1, high methylation level of nucleotide site of NNMT gene, and/or high methylation level of DNA CpG site of NNMT gene.

In another preferred embodiment, the instruction or label records that the compound of formula I, or an optical isomer thereof, or a racemate thereof, or a solvate thereof, or a pharmaceutically acceptable salt thereof, or a deuterated compound thereof according to the first aspect of the present invention is not suitable for use in the prevention and/or treatment of patient tumor with high expression or high activity of mitochondria permeability transition pore, high expression or high activity of peptidyl-prolyl cis-trans isomerase F, high expression of NNMT gene, low expression of DNA methylase, low expression of UHRF1, low methylation level of nucleotide site of NNMT gene, and/or low methylation level of DNA CpG site of NNMT gene.

It should be understood that, in the present invention, each of the technical features specifically described above and below can be combined with each other, thereby constituting new or preferred technical solutions which need not be redundantly described one-by-one.

### DESCRIPTION OF THE DRAWINGS

FIG. 1 shows the expression level of PPIF protein using Western Blot test, wherein, Con shRNA refers to the expression level of PPIF protein in Daoy cell transfected with empty virus vector without carrying shRNA that can specifically induce the degradation of PPIF mRNA; PPIF shRNA refers to the expression level of PPIF protein in Daoy cell transfected with virus vector carrying shRNA that can specifically induce the degradation of PPIF mRNA.

FIG. 2 shows the relative cell viability of Daoy cell having inactive mPTP and Daoy cell having active mPTP, wherein, Con shRNA refers to the relative viability of Daoy cell transfected with empty virus vector without carrying shRNA that can specifically induce the degradation of PPIF mRNA; PPIF shRNA refers to the relative viability of Daoy cell transfected with virus vector carrying shRNA that can specifically induce the degradation of PPIF mRNA.

FIG. 3 shows the expression content of NNMT protein in Con-NCI-H82 cell and ov-NNMT NCI-H82 cell using Western Blot test, wherein, Con-NCI-H82 refers to the expression content of NNMT protein in NCI-H82 cell transfected with empty virus vector without carrying NNMT gene and shRNA, which is used as control; ov-NNMT NCI-H82 refers to the expression content of NNMT protein in NCI-H82 cell transfected with virus vector carrying NNMT gene.

FIG. 4 shows the expression content of DNMT1 protein in Con-NCI-H82 cell and sh-DNMT1 NCI-H82 cell using Western Blot test, wherein, Con-NCI-H82 refers to the expression content of DNMT1 protein in NCI-H82 cell transfected with empty virus vector without carrying NNMT gene and shRNA, which is used as control; sh-DNMT1 NCI-H82 refers to the expression content of DNMT1 protein in NCI-H82 cell transfected with virus vector carrying shRNA.

FIG. 5 shows the relative cell viability of Con-NCI-H82 cell and ov-NNMT NCI-H82 cell, wherein, Con-NCI-H82 refers to the cell viability of NCI-H82 cell transfected with empty virus vector without carrying NNMT gene and shRNA, which is used as control; ov-NNMT NCI-H82 refers to the cell viability of NCI-H82 cell transfected with virus vector carrying NNMT gene.

FIG. 6 shows the relative cell viability of Con-NCI-H82 cell and sh-DNMT1 NCI-H82 cell, wherein, Con-NCI-H82 refers to the cell viability of NCI-H82 cell transfected with empty virus vector without carrying NNMT gene and shRNA, which is used as control; sh-DNMT1 NCI-H82 refers to the cell viability of NCI-H82 cell transfected with virus vector carrying shRNA.

FIG. 7 shows the correlation between the expression of NNMT and the expression of DNMT1, UHRF1, DNMT3a and DNMT3b in tumor cells.

FIG. 8 shows the expression of NNMT gene in tumor cells sensitive and insensitive to the compounds of the present invention.

FIG. 9 shows the methylation level of DNA CpG site of promoter region of NNMT gene in tumor cells sensitive and insensitive to the compounds of the present invention.

FIG. 10 shows the methylation level of DNA CpG sites from 1050 bp before the transcription start site to 499 bp after the transcription start site in NNMT gene in tumor cells sensitive and insensitive to the compounds of the present invention.

FIG. 11 shows the methylation level of DNA CpG sites from 1050 bp to 193 bp before the transcription start site in NNMT gene in tumor cells sensitive and insensitive to the compounds of the present invention.

FIG. 12 shows the methylation level of DNA CpG sites of specific NNMT gene (ie, site 114165695, site 114165730, site 114165769, site 114165804, site 114165938, site 114166050, site 114166066 on human chromosome 11) in tumor cells sensitive and insensitive to the compounds of the present invention, black dot indicates that the relevant sites are methylated, white white dot indicates that the relevant sites are not methylated, SST refers to the transcription start site, and Chr11 refers to human chromosome 11 according to human genome version GCF_00000 1405.25 (GRCh37. p13).

### EMBODIMENTS FOR CARRYING OUT THE INVENTION

Based on an extensive and intensive research, the inventors have unexpectedly found the compound of the present invention has excellent precision treatment effect on tumor with low expression, no expression, low activity or no activity of mitochondria permeability transition pore, low expression, no expression, low activity or no activity of peptidyl-prolyl cis-trans isomerase F, low or no expression of NNMT gene, high expression of DNA methylase, high expression of UHRF1, high methylation level of nucleotide site of NNMT gene, and/or high methylation level of DNA CpG site of NNMT gene. On this basis, the inventors has completed the present invention.

### Terms

As used herein, the term “comprise”, “comprising”, and “containing” are used interchangeably, which not only comprise closed definitions, but also semi-closed and open definitions. In other words, the term comprises “consisting of” and “essentially consisting of”.

As used herein, the term “anti-tumor cancer” and “anti-tumor drug” are used interchangeably.

As used herein, the term “cancer” and “tumor” are used interchangeably.

As used herein, the term “a cell” refers to a cell (e.g. single tumor cell) or a group of cells containing multiple similar cells ((e.g. a tumor tissue).

As used herein, “the compound of present invention is suitable for use in the prevention and/or treatment of patient tumor” comprises “patient tumor is sensitive to compound of present invention”.

As used herein, “the compound of present invention is not suitable for use in the prevention and/or treatment of patient tumor” comprises “patient tumor is not sensitive to compound of present invention”.

As used herein, the term “high methylation level of DNA CpG site”, “high level of DNA CpG site methylation” and “high methylation of DNA CpG site” are used interchangeably.

As used herein, the term “low methylation level of DNA CpG site”, “low level of DNA CpG site methylation” and “low methylation of DNA CpG site” are used interchangeably.

As used herein, the term “methylation of CpG site”, “methylation of CpG nucleotide” and “CpG methylation” are used interchangeably.

As used herein, the term “IC50” and “IC₅₀” are used interchangeably, which refers to 50% inhibiting concentration, ie, the concentration of the inhibitor when 50% inhibitory effect is achieved.

As used herein, the term “methylation of CpG site”, “methylation of CpG nucleotide” and “CpG methylation” are used interchangeably.

As used herein, the term “P/S” refers to adding penicillin and streptomycin into the culture medium.

As used herein, mitochondria permeability transition pore is abbreviated as mPTP.

As used herein, peptidyl-prolyl cis-trans isomerase F is abbreviated as PPIF.

As used herein, the term “NNMT” refers to Nicotinamide N-Methyltransferase.

As used herein, the term “bp” refers to base pair.

As used herein, the term “SST” refers to the transcription start site.

As used herein, the term “Chr11” refers to human chromosome 11 according to human genome version GCF_000001405.25 (GRCh37. p13).

As used herein, the term “human chromosome 11” refers to human chromosome 11 according to human genome version GCF_000001405.25 (GRCh37. p13).

As used herein, the terms “before the transcription start site” and “after the transcription start site” do not comprise the transcription start site itself.

As used herein, the terms “site 114165695 on human chromosome 11” refers to nucleotide of site 114165695 on human chromosome 11, and the like.

As used herein, S-adenosyl methionine is abbreviated as SAM.

As used herein, the gene expression comprises the protein expression of the gene and/or the mRNA expression of the gene.

As used herein, the term “DNMT3a” and “DNMT3A” are used interchangeably, which refers to DNA methyltransferase 3a.

As used herein, the term “DNMT3b” and “DNMT3B” are used interchangeably, which refers to DNA methyltransferase 3b.

As used herein, the term “DNMT1” refers to DNA methyltransferase 1.

As used herein, the term “UHRF1” refers to ubiquitin-like with PHD and ring finger domain 1.

As used herein, the term “SF126” and “SF-126” are used interchangeably.

As used herein, the term “deuterated” refers to one or more hydrogen atoms in a compound or group are substituted by deuterium. The deuterated can be mono-substituted, di-substituted, multi-substituted or fully-substituted.

As used herein, the term “solvate” refers to a complex formed by the coordination of a compound with a solvent molecule in a specific ratio.

As used herein, the term “MS-ESI” refers to Electro Spray Ionization-Mass Spectroscopy.

As used herein, the term “1H NMR” refers to H Nuclear Magnetic Resonance Spectra.

It should be understood that the skilled in the art can choose the substituents and substituted forms on the compound of the present invention to obtain chemically stable compounds, the compound can be synthesized by the techniques known in the art and the methods described below.

If the compound is substituted by more than one substituents, it should be understood that the substituents can be on the same carbon or different carbons, as long as a stable structure is obtained.

As used herein, the term “substitute” or “substituted” means the hydrogen atom on the group is substituted by non-hydrogen atom group, but it needs to meet its valence requirements and the substituted compound is chemically stable, that is, the substituted compound does not spontaneously undergo transformations such as cyclization and elimination, etc.

As used herein, “R₁”, “R1” and “R¹” have the same meaning and can be used interchangeably, the other similar definitions have the same meaning.

As used herein, “—” denotes the linking site of the group.

As used herein, the term “alkyl” refers to a saturated hydrocarbon group with linear chain (ie, unbranched chain) or branched chain, or a combination of linear and branched chains, the alkyl only have carbon and hydrogen atoms. When the number of carbon atoms is limited in front of the alkyl (e.g. C1-C6 alkyl), it refers to the number of carbon atoms (e.g. 1-6) in the alkyl, for example, C1-C4 alkyl refers to an alkyl having 1-4 carbon atoms. Representative examples comprise but are not limited to methyl, ethyl, propyl, isopropyl, butyl, isobutyl, sec-butyl, tert-butyl, or the like.

As used herein, the term “halogen” refers to F, Cl, Br or I.

As used herein, the term “halo” means the group is substituted by halogen.

As used herein, the term “haloalkyl” means that one or more (preferably 1, 2, 3 or 4) hydrogens on the alkyl are substituted by halogen, the alkyl and halogen are as defined above.

When the number of carbon atoms is limited in front of the haloalkyl (e.g. C1-C8 haloalkyl), it refers to the number of carbon atoms (e.g. 1-8) in the haloalkyl, for example, C1-C6 haloalkyl refers to an haloalkyl having 1-6 carbon atoms. Representative examples comprise but are not limited to —CF₃, —CHF₂, monofluoroisopropyl, difluorobutyl, or the like.

As used herein, the term “cycloalkyl” refers to a cyclic group having a saturated or partially saturated monocyclic ring, bicyclic ring or polycyclic ring (fused ring, bridged ring or spiro ring) hydrocarbon group. When the number of carbon atoms is limited in front of the cycloalkyl (e.g. C3-C12 cycloalkyl), it refers to the number of ring carbon atoms (e.g. 3-12) in the cycloalkyl. For example, C3-C8 cycloalkyl refers to a saturated or partially saturated monocycloalkyl or dicycloalkyl having 3-8 ring carbon atoms, comprising cyclopropyl, cyclobutyl, cyclopentyl, cycloheptyl, or the like. The term “spirocycloalkyl” refers to a bicyclic or polycyclic group that shares a carbon atom (referred to spiro-atom) between single rings, the spirocycloalkyl can have one or more double bonds, but no ring has a fully conjugated R electron system. Fused cycloalkyl refers to all carbon bicyclic or polycyclic group in which each rings in the system shares an adjacent pair of carbon atoms with other rings, one or more rings can have one or more double bonds, but no ring has a fully conjugated π electron system. Bridged cycloalkyl refers to all carbon polycyclic group in which any two rings share two non-directly connected carbon atoms, the bridged cycloalkyl can have one or more double bonds, but no ring has a fully conjugated 11 electron system. The representative examples of cycloalkyl comprise but are not limited to

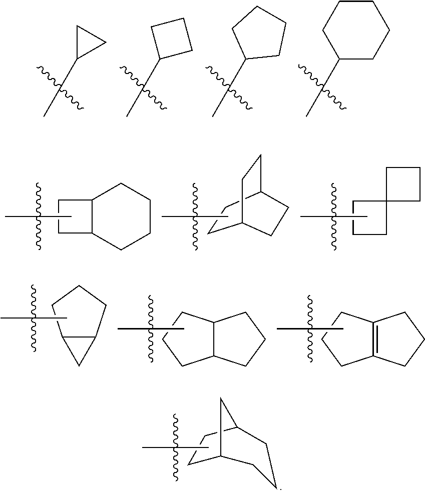

As used herein, the term “halocycloalkyl” means that one or more (preferably 1, 2, 3 or 4) hydrogens on cycloalkyl are substituted by halogen, the cycloalkyl and halogen are as defined above, When the number of carbon atoms is limited in front of the halocycloalkyl (e.g, C3-C8 halocycloalkyl), it refers to the number of ring carbon atoms (e.g. 3-8) in the halocycloalkyl, for example, C3-C8 halocycloalkyl refers to an halocycloalkyl having 3-8 ring carbon atoms. Representative examples comprises but are not limited to monofluorocyclopropyl, monochlorocyclobutyl, monofluorocyclopentyl, difluorocycloheptyl, or the like.

As used herein, the term “alkoxyl” refers to R—O— group, wherein R is alkyl, the alkyl is as defined above. When the number of carbon atoms is limited in front of the alkoxyl, for example, C1-C8 alkoxyl means that the alkyl in the alkoxyl has 1-8 carbon atoms. Representative examples of alkoxyl comprise but are not limited to methoxyl, ethoxyl, n-propoxyl, isopropoxyl, tert-butoxyl, or the like.

As used herein, the term “alkylthio” refers to R—S— group, wherein R is alkyl, the alkyl is as defined above. When the number of carbon atoms is limited in front of the alkylthio, for example, C1-C8 alkylthio means that the alkyl in the alkylthio has 1-8 carbon atoms. Representative examples of alkylthio comprise but are not limited to methylthio, ethylthio, n-propylthio, isopropylthio, tert-butylthio, or the like.

As used herein, the term “haloalkoxyl” refers to haloalkyl-O—, wherein the haloalkyl is as defined above. When the number of carbon atoms is limited in front of the haloalkoxyl, for example, C1-C6 haloalkoxyl refers to C1-C6 haloalkyl-O—, ie, the haloalkoxyl having 1-6 carbon atoms. Representative examples comprise but are not limited to monofluoromethoxyl, monofluoroethoxyl, bisfluorobutoxyl, or the like.

As used herein, the term “haloalkylthio” refers to haloalkyl-S—, wherein the haloalkyl is as defined above. When the number of carbon atoms is limited in front of the haloalkylthio, for example, C1-C6 haloalkylthio refers to C1-C6 haloalkyl-S—, ie, the haloalkylthio having 1-6 carbon atoms. Representative examples of haloalkylthio comprise but are not limited to monofluoromethylthio, monofluoroethylthio, difluorobutylthio, or the like.

As used herein, the term “cycloalkoxyl” refers to R—O—, wherein R is cycloalkyl, the cycloalkyl is as defined above. When the number of carbon atoms is limited in front of the cycloalkoxyl, for example, C3-C8 cycloalkoxyl means that the cycloalkyl in the cycloalkoxyl has 3-8 ring carbon atoms. Representative examples of cycloalkoxyl comprise but are not limited to cyclopropyloxyl, cyclobutoxyl, or the like.

As used herein, the term “cycloalkylthio” refers to R—S— group, wherein R is cycloalkyl, the cycloalkyl is as defined above. When the number of carbon atoms is limited in front of the cycloalkylthio, for example, C3-C8 cycloalkylthio means that the cycloalkyl in the cycloalkylthio has 3-8 ring carbon atoms. Representative examples of cycloalkylthio comprise but are not limited to cyclopropylthio, cyclobutythio, or the like.

As used herein, the term “halocycloalkoxyl” means that one or more (preferably 1, 2, 3 or 4) hydrogens on the cycloalkoxyl are substituted by halogen, the cycloalkoxyl and halogen are as defined above. When the number of carbon atoms is limited in front of the halocycloalkoxyl (e.g. C3-C8 halocycloalkoxyl), it refers to the number of ring carbon atoms (e.g. 3-8) in the halocycloalkoxyl, for example, C3-C8 halocycloalkoxyl refers to an halocycloalkoxyl having 3-8 ring carbon atoms. Representative examples comprises but are not limited to monofluorocyclopropyl-O—, monochlorocyclobutyl-O—, monofluorocyclopentyl-O—, difluorocycloheptyl-O—, or similar functional groups, or the like.

As used herein, the term “halocycloalkylthio” means that one or more (preferably 1, 2, 3 or 4) hydrogens on the cloalkylthio are substituted by halogen, the cycloalkoxyl and halogen are as defined above. When the number of carbon atoms is limited in front of the halocycloalkylthio (e.g. C3-C8 halocycloalkylthio), it refers to the number of ring carbon atoms (e.g. 3-8) in the halocycloalkylthio, for example, C3-C8 halocycloalkylthio refers to an halocycloalkylthio having 3-8 ring carbon atoms. Representative examples comprises but are not limited to monofluorocyclopropyl-S—, monochlorocyclobutyl-S—, monofluorocyclopentyl-S—, difluorocycloheptyl-S—, or similar functional groups, or the like.

As used herein, the term “heterocycloalkane ring” refers to fully saturated or partially unsaturated ring (comprising but not limited to such as 3-7 membered monocyclic ring, 7-11 membered bicyclic ring, or 8-16 membered tricyclic ring), at least one heteroatom is present in a ring with at least one carbon atom. When the number of members is limited in front of the heterocycloalkane ring, it refers to the number of ring atoms in the heterocycloalkane ring, for example, 3-16 membered heterocycloalkane ring refers to a heterocycloalkane ring having 3-16 ring atoms. Each heterocyclic ring having heteroatoms can have one or more (e.g. 1, 2, 3 or 4) heteroatoms, each of heteroatoms is independently selected from the group consisting of nitrogen atom, oxygen atom or sulfur atom, wherein the nitrogen atom or sulfur atom can be oxidized, and the nitrogen atom can also be quaternized. Representative examples of monocyclic heterocycloalkane ring comprise but are not limited to azetidine ring, oxetane ring, tetrahydrofuran ring, piperidine ring, piperazine ring. Polycyclic heterocycloalkane ring comprises spiro, fused and bridged ring, the spiro, fused and bridged heterocycloalkane ring is optionally linked with other rings by single bond, or further linked with other cycloalkane ring and heterocycloalkane ring by any two or more atoms on the ring.

As used herein, the term “heterocycloalkyl” refers to fully saturated or partially unsaturated cyclic (comprising but not limited to such as 3-7 membered monocyclic ring, 7-11 membered bicyclic ring, or 8-16 membered tricyclic ring) group, at least one heteroatom is present in a ring with at least one carbon atom, the linking site of the group is located on the ring having heteroatoms. When the number of members is limited in front of the heterocycloalkyl, it refers to the number of ring atoms in the heterocycloalkyl, for example, 3-16 membered heterocycloalkyl refers to a heterocycloalkyl having 3-16 ring atoms. Each heterocyclic ring having heteroatoms can have one or more (e.g. 1, 2, 3 or 4) heteroatoms, each of heteroatoms is independently selected from the group consisting of nitrogen atom, oxygen atom or sulfur atom, wherein the nitrogen atom or sulfur atom can be oxidized, and the nitrogen atom can also be quaternized.

Representative examples of monocyclic heterocycloalkyl comprise but are not limited to azetidinyl, oxetanyl, tetrahydrofuranyl, piperidinyl, piperazinyl. Polycyclic heterocycloalkyl comprises spiro, fused and bridged heterocyclyl, the spiro, fused and bridged heterocycloalkyl is optionally linked with other groups by single bond, or further linked with other cycloalkane rings and heterocycloalkane ring by any two or more atoms on the ring.

As used herein, the term “aryl” refers to an full carbon monocyclic ring or fused polycyclic ring (i.e., a ring that shares adjacent carbon atom pairs) group with a conjugated π electron system, which is aromatic cyclic hydrocarbon compound group. When the number of carbon atoms is limited in front of the aryl, it refers to the number of ring carbon atoms in the aryl, for example, C6-C12 aryl means that the aryl has 6-12 ring carbon atoms, such as phenyl and naphthyl.

As used herein, the term “heteroaryl” refers to aromatic heterocyclic ring group having one to more (preferably 1, 2, 3 or 4) heteroatoms, at least one heteroatom is present in a ring with at least one carbon atom. The heteroaryl can be monocyclic ring, or polycyclic ring (bicyclic, tricyclic or polycyclic ring) fused together or covalently connected. Each of heterocyclic ring having heteroatom can have one or more (e.g. 1, 2, 3, 4) heteroatoms independently selected from the group consisting of oxygen, sulfur and nitrogen. When the number of members is limited in front of the heteroaryl, it refers to the number of ring atoms in the heteroaryl, for example, 5-12 membered heteroaryl refers to a heteroaryl having 5-12 ring atoms. Representative examples comprise but are not limited to pyrrolyl, pyrazolyl, imidazolyl, thiazolyl, furanyl, pyridyl, pyrimidinyl, etc.

As used herein, the term “carboxyl” refers to —COOH or -alkyl-COOH, the alkyl is as defined above. For example, “C2-C4 carboxyl” refers to —C1-C3 alkyl-COOH. Representative examples of carboxyl comprise but are not limited to —COOH, —CH₂COOH, or the like.

As used herein, the term “ester group” refers to R—C(O)—O— or —C(O)—O—R, wherein, R is alkyl, the alkyl is defined as above. For example, “C₂-C₄ ester group” refers to C₁-C₃ alkyl-C(O)—O— or —C(O)—O—C₁-C₃ alkyl. Representative examples of ester group comprise but are not limited to CH₃C(O)O—, C₂H₅C(O)O—, (CH₃)₂CHC(O)O—, —C(O)OCH₃, —C(O)OC₂H₅, or the like.

As used herein, the term “amide group” refers to R—C(O)—NH— or —C(O)—NH—R, wherein, R is alkyl, the alkyl is defined as above. For example, “C₂-C₄ amide group” refers to C₁-C₃ alkyl-C(O)—NH— or —C(O)—NH—C₁-C₃ alkyl. Representative examples of amide group comprise but are not limited to CH₃C(O)—NH—, C₂H₅C(O)—NH—, (CH₃)₂CHC(O)—NH—, —C(O)—NH—CH₃, —C(O)—NH—C₂H₅, or the like.

As used herein, the term —C(O)— and

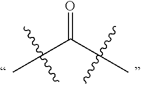 [SMILES: *C(*)=O]

are used interchangeably.

As used alone or as part of other substituent, the term “amino” refers to —NH₂.

As used alone or as part of other substituent, the term ‘nitro’ refers to —NO₂.

As used alone or as part of other substituent, the term “cyano” refers to —CN.

As used alone or as part of other substituent, the term “hydroxyl” refers to —OH.

As used alone or as part of other substituent, the term “sulfhydryl” refers to —SH.

As used alone or as part of other substituent, the term “trifluoromethyl” refers to

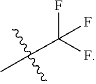 [SMILES: *C(F)(F)F]

As used alone or as part of other substituent, the term “trifluoromethoxyl” refers to

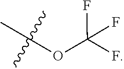 [SMILES: *OC(F)(F)F]

In present invention, it should be understood that all substituents are unsubstituted, unless explicitly described herein as “substituted”. The specific substituent is the substituent as described above or the substituent in each Example. The “substituted” means that one or more hydrogen atoms on specific group are substituted by specific substituent. Preferably, each “substituted” means that one or more (preferably 1, 2, 3, 4, 5, 6, 7, or 8) hydrogen atoms on the ring or group are independently substituted by substituent selected from the group consisting of C1-C10 alkyl, C3-C8 cycloalkyl, C1-C10 haloalkyl, C3-C8 halocycloalkyl, C3-C8 cycloalkoxyl, C3-C8 cycloalkylthio, C3-C8 halocycloalkoxyl, C3-C8 halocycloalkylthio, halogen, nitro, —CN, hydroxyl, sulfhydryl, amino, C1-C4 carboxyl, C2-C8 ester group, C2-C4 amide group, C1-C10 alkoxyl, C1-C10 alkylthio, C1-C10 haloalkoxyl, C1-C10 haloalkylthio, C6-C12 aryl, 5-12 membered heteroaryl, 3-8 membered heterocycloalkyl. Unless otherwise specified, each substituted group can have a substituent selected from a specified group at any substituted position of the group, the substitution can be the same or different at each substituted position.

In the present invention, the term “prevention” refers to a method of preventing the occurrence of disease and/or its accompanying symptoms, or protecting a subject from getting disease. The term ‘prevention’ also comprises delaying the occurrence of disease and/or its accompanying symptoms and reducing the risk of getting disease for subject.

In the present invention, the term “treatment” comprises delaying and terminating the progression of the disease, or eliminating the disease, and it does not require 100% inhibition, elimination and reversal. In some embodiments, compared to the level observed in the absence of the compound of present invention, the compound of present invention alleviates, inhibits and/or reverses related disease (e.g. tumor) and its accompanying symptoms, for example, by at least about 10%, at least about 30%, at least about 50%, at least about 80%, at least about 90%, or 100%.

### Compound

As used herein, the terms “compound of the present invention”, “compound of formula I of the present invention” and “compound of formula I” are used interchangeably, and refer to a compound of formula I, or an optical isomer thereof, or a racemate thereof, or a solvate thereof, or a pharmaceutically acceptable salt thereof, or a deuterated compound thereof.

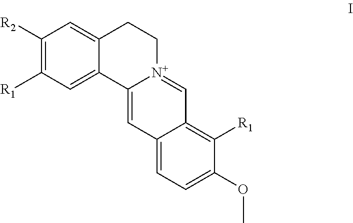

Specifically, the compound of formula I, or an optical isomer thereof, or a racemate thereof, or a solvate thereof, or a pharmaceutically acceptable salt thereof, or a deuterated compound thereof is as described above in the first aspect of the present invention.

The compound of formula I of the present invention can be prepared using the known organic synthesis method in the art. Preferably, the compound of the present invention is the compound prepared in the Examples of the present invention (comprising salt thereof or free form without salt radical) prepared in the Examples of the present invention.

The term “pharmaceutically acceptable salt” refers to a salt formed by the compound of the present invention and an acid or a base, the salt is suitable for use as a drug. The compound of formula I in present invention can be converted into its pharmaceutically acceptable salt using conventional methods. For example, a solution of corresponding acid can be added into the solution of above compounds, and the solvent is removed after the salt is formed, thereby forming the corresponding salt of the compound of the present invention.

### Mitochondria Permeability Transition Pore

In present invention, the mitochondria permeability transition pore is abbreviated as mPTP.

The compound of present invention has excellent precision treatment effect on tumor with low expression, no expression, low activity or no activity of mitochondria permeability transition pore, the tumor with low expression, no expression, low activity or no activity of mitochondria permeability transition pore is sensitive to the compound of present invention.

### Peptidyl-Prolyl Cis-Trans Isomerase F

In present invention, the peptidyl-prolyl cis-trans isomerase F is abbreviated as PPIF.

The compound of present invention has excellent precision treatment effect on tumor with low expression, no expression, low activity or no activity of peptidyl-prolyl cis-trans isomerase F, the tumor with low expression, no expression, low activity or no activity of peptidyl-prolyl cis-trans isomerase F is sensitive to the compound of present invention.

### NNMT Gene

In the present invention, the English name of NNMT is Nicotinamide N-Methyltransferase. Different databases have different identification numbers for NNMT gene as follows: HGNC: 7861; Entrez Gene: 4837; Ensembl: ENSG00000166741; OMIM: 600008; UniProtKB: P40261

According to version GCF_000001405.25 (GRCh37.p13) of human genome, the NNMT gene is located at 114,128,528 bp to 114,184,258 bp on human chromosome 11, the total length of DNA sequence of NNMT gene is 55,731 bp, the NNMT gene comprises promoter region, exon region and intron region, the transcription start site of NNMT gene is at site 114,166,535 bp.

The promoter region of NNMT gene is the nucleotide sequence from the 114164535 bp to 114167034 bp on human chromosome 11, i.e. the sequence from 2000 bp before the transcription start site (bold section) and 499 bp after the transcription start site (non-bold section) in NNMT gene, the total length of promoter region of NNMT gene is 2500 bp, The nucleotide sequence of the promoter region of NNMT gene is as shown in SEQ ID NO: 1 as follows: SEQ ID NO: 1:

SEQ ID NO: 1:
TATCCAAGAGCTATCAGCACTCCCATGTTTATTGTAGCACTGTTCACAA
TAGCCAAGATTTGGAAGTACTCTAAGTGTCCATTAGCAGATGAATGGAT
AAAGACAATGTGGTAATACACATAATGGAGTACTATTCAGTCATAAAGA
AGAATTAGATCCTGTCATTTGCAATAACATGGATGGAACTGGAGGTCAT
AATGTTGAGTGAAATAAACCAGGCACAGAAAGACAAACTTTGCATGTTC
TCACTTATTTATGGGAGCTAAAAACTAAAATAACTGAACTCACAGAGAT
AGAGAGTAGAAGGATGGTTACGAGAGGATGGGAAGGGTAGCGAGGTGGG
TAGGGGGGATGTGGGGATCATTAATGGGTATAAAAAATAGTTAGAGGCC
AGGCGCAGTGGCTCACGCCTGTAATCCCAGCACTTTGGGAGGCCGAGGT
AGGCGGAACACCTGAGGAGTTCAAGACCAGCCTGGCCAATATGATGAAA
CCCCGTCTCTACTAAAAATACAAAAATTAGCTGGGCGTGATGGTGTGCA
CCTGTAGTCCCAGCTGCTTGGGAGGCTGAGGCAGGAGAATCGCTGGAAC
CCAAGAGGTGAAGGTTGCAGTGAGCTGAGATCGCGTCACTGCACTCCAG
CCTGGGTGACAGAGTGAGACTCCACATCAAAAAAAAAAAAAAAAAGTTA
GAAAGATTGAATAAGACCTAATATTTGCTAGCACAACAGGGTGAATATA
GTAAAAAATAATTTATTTGTACCTTCAAAAATAACTAGACAAGTATAAT
TGGGTTGTTTGTAACACACAAAAAATAAGTACTTGAAGTGGTGGATACC
CCATTTACCCTGATGTGATTATTTTGTATTGCAGGCCTCTATCAGAATA
TCTCATGTAACCCATAAATATATACACCTACTCTGTACCCACAAAAAGT
TTTTAAAAAGAAAAATAAATAGCAACCGAAAAAAAAAGAGAGGGAGAAA
AGAAAAAAGAAAAAAAAATCAAGTGCCTGGCTGGGTAGAATAAATTCTA
AGGCCACAATGTTACTGACCATGGGTTTTTTGGCTCTCAGTGTATAGAA
ATTGACACAAGGCCAATAGTCTTCCCAAACATGCTTTACTGGAACTTAC
GCCCTGGCATAAGGGCCACAACAAAAGAGAGAGCGAATTCTCTGGCTTG
CTGACTCCTTGGAAAAAACCGGTAGGGATTTTTTTATTAGGCAAAGCAC
AGGAATTGACGTCAGAGGCAGGATGTGCTGCTGGGCAAAGCATACGAGA
AGTGGGGTATGCAGGTCAGCATTACTTGGTTGCAATGGTTATCTTGAGG
AATGGGCCAACTGGTGGTCTGGCCAGTGGCAACAAGGCTGTAAATCAAT
TATTCAGCATTCCTTCCCAAGGTGGGACACCCGGCAACATTGTTTATCT
CCTAAGGCCAGTTCCTGGAATTAAGTGAAAGGATGACTAATGGACATGT
TGTCAGTGAGGTAGTGGTGTGGGTTTTGTGACCAGTGGGAATGCACGAA
AGAATGCTTTAGCGGGGAGTGAGCTGAAGCCAAGCCCCATCCCTACTCT
GTCTCAAAGTGAGTTCAGAAAAGGGGATTTAAAGAATTCTTTTTTTTTT
TTTTTTTTTTTTTGAGACAGAGTCTTGCTCTGTCGCCCAGGCTGGAGTG
CAGTGGCGCCATCTTGGCTCACTGCAAGCTCCGCCCCCCGGGTTCATGC
CATTCTCCTGCCTCAGCCTCCCAAGTAGCTGGGACTGCAGGTGCCTACC
ACCAAGCCCAGCTAATTTTTTGTATTTTTTTTTTAGTAGAGACGGGGTT
TCACCATGTTAGCCAGGATGGTCTCGATCTCCTGACCTCGTGATCTGCC
CGCCTTAGCCTCCCAAAGTGCTGGGATTACAGGCATGAGCCTCCGCCCC
CGGCCTTAAATAATTCTTAAAGGAAGTAAAGTTAACTTTGAAAGAACTA
TCAGGATTTGGATTGACTGAAAGGAGTGGGGAAGCTTAGGGAGGAGGTG
CTTGCCAGACACTGGGTCATGGCAGTGGTCGGTGAAGCTGCAGTTGCCT
AGGGCAGGGATGGAGAGAGAGTCTGGGCATGAGGAGAGGGTCTCGGGAT
GTTTGGCTGGACTAGATTTTACAGAAAGCCTTATCCAGGCTTTTAAAAT
TACTCTTTCCAGACTTCATCTGAGACTCCTTCTTCAGCCAACATTCCTT
AGCCCTGAATACATTTCCTATCCTCATCTTTCCCTTCTTTTTTTTCCTT
TCTTTTACATGTTTAAATTTAAACCATTCTTCGTGACCCCTTTTCTTGG
GAGATTCATGGCAAGAACGAGAAGAATGATGGTGCTTGTTAGGGGATGT
CCTGTCTCTCTGAACTTTGGGGTCCTATGCATTAAATAATTTTCCTGAC
GAGCTCAAGTGCTCCCTCTGGTCTACAATCCCTGGCGGCTGGCCTTCAT
CCCTTGGGCAAGCATTGCATACAGCTCATGGCCCTCCCTCTACCATACC
C.

In present invention, the site 114165695, site 114165730, site 114165769, site 114165804, site 114165938, site 114166050 and site 114166066 on the human chromosome 11 correspond to the nucleotide site in SEQ ID NO: 1 as shown in Table 1:

TABLE 1

| | Corresponding to the |
|---|---|
| Site on the human | nucleotide site in |
| chromosome 11 | SEQ ID NO: 1 |
| site 114165695 | site 1161 |
| site 114165730 | site 1196 |
| site 114165769 | site 1235 |
| site 114165804 | site 1270 |
| site 114165938 | site 1404 |
| site 114166050 | site 1516 |
| site 114166066 | site 1532 |

### DNA Methylation

DNA methylation is a form of chemical modification of DNA, which can change genetic performance under no change of DNA sequence.

Typically, DNA methylation is the methylation of DNA CpG site. The distribution of CpG binucleotide is very uneven in the human genome, while CpG remains or is higher than normal level in some regions of the genome. The CpG site rich region (also known as CpG island) is mainly located in the promoter region and exon regions of the gene, which is a region rich in CpG dinucleotide. About 60% of the promoters of the gene contains CpG island. The CpG is the abbreviation of cytosine (C)-phosphate (p)-guanine (G).

### Tumor

As used herein, the term “tumor” and “cancer” are used interchangeably.

In a preferred embodiment of the present invention, the tumor comprises tumor with low expression, no expression, low activity or no activity of mitochondria permeability transition pore. Typically, the tumor with low expression, no expression, low activity or no activity of mitochondria permeability transition pore is as described above in the third aspect of the present invention.

In a preferred embodiment of the present invention, the tumor comprises tumor with low expression, no expression, low activity or no activity of peptidyl-prolyl cis-trans isomerase F.

Typically, the tumor with low expression, no expression, low activity or no activity of peptidyl-prolyl cis-trans isomerase F is as described above in the third aspect of the present invention.

In a preferred embodiment of the present invention, the tumor comprises tumor with low or no expression of NNMT gene. Typically, the tumor with low or no expression of NNMT gene is as described above in the third aspect of the present invention.

In a preferred embodiment of the present invention, the tumor comprises tumor with high expression of DNA methylase. Typically, the tumor with high expression of DNA methylase is as described above in the third aspect of the present invention.

The DNA methylase of present invention comprises but is not limited to DNMT1, DNMT3a, DNMT3b, and combinations thereof. Preferably, the DNA methylase comprises DNMT1.

In a preferred embodiment of the present invention, the tumor comprises tumor with high expression of DNMT1. Typically, the tumor with high expression of DNMT1 is as described above in the third aspect of the present invention.

In a preferred embodiment of the present invention, the tumor comprises tumor with high expression of DNMT3a. Typically, the tumor with high expression of DNMT3a is as described above in the third aspect of the present invention.

In a preferred embodiment of the present invention, the tumor comprises tumor with high expression of DNMT3b. Typically, the tumor with high expression of DNMT3b is as described above in the third aspect of the present invention.

In a preferred embodiment of the present invention, the tumor comprises tumor with high expression of UHRF1 (ubiquitin-like with PHD and ring finger domain 1). Typically, the tumor with high expression of UHRF1 is as described above in the third aspect of the present invention.

In a preferred embodiment of the present invention, the tumor comprises tumor with high methylation level of nucleotide site of NNMT gene. Typically, the tumor with high methylation level of nucleotide site of NNMT gene is as described above in the third aspect of the present invention.

In a preferred embodiment of the present invention, the tumor comprises tumor with high methylation level of DNA CpG site of NNMT gene. Typically, the tumor with high methylation level of DNA CpG site of NNMT gene is as described above in the third aspect of the present invention.

Typically, the tumor of present invention is as described above in the third aspect of the present invention.

In present invention, the type of tumors corresponding to tumor cell lines are shown in Table 2

TABLE 2

| Tumor cell line | The corresponding type of tumor |
|---|---|
| NCI-H82 | Human small cell lung cancer cell |
| G-401 | Human renal carcinoma Wilms cell |
| MDA-MB-453 | Breast cancer cell |
| SW48 | Human colon adenocarcinoma cell |
| GB-1 | Human brain glioblastoma cell |
| CFPAC-1 | Human pancreatic cancer cell |
| SF126 | Human glioblastoma multiforme cell |
| 786-O | Clear cell renal cell adenocarcinoma cell |

### Anti-Tumor Drug

The anti-tumor drug can be the compound of formula I, or an optical isomer thereof, or a racemate thereof, or a solvate thereof, or a pharmaceutically acceptable salt thereof, or a deuterated compound thereof of the present invention.

### Use

A use of the compound of formula I of the present invention for preventing and/or treating tumor is provided.

Specifically, the compound of present invention has more remarkable and excellent precision treatment effect on tumor with low expression, no expression, low activity or no activity of mitochondria permeability transition pore, low expression, no expression, low activity or no activity of peptidyl-prolyl cis-trans isomerase F, low or no expression of NNMT gene, high expression of DNA methylase, high expression of UHRF1, high methylation level of nucleotide site of NNMT gene, and/or high methylation level of DNA CpG site of NNMT gene.

The present invention further provides a method for preventing and/or treating tumor, which comprises administering the compound of the present invention to a subject in need. Therefore, during the prevention and/or treatment of tumor, firstly administering mitochondria permeability transition pore inhibitor, peptidyl-prolyl cis-trans isomerase F inhibitor, NNMT gene inhibitor, DNA methylase promoter, UHRF1 promoter, methylation promoter of nucleotide site of NNMT gene, and/or methylation promoter of DNA CpG site of NNMT gene to achieve low expression, no expression, low activity or no activity of mitochondria permeability transition pore, low expression, no expression, low activity or no activity of peptidyl-prolyl cis-trans isomerase F, low or no expression of NNMT gene, high expression of DNA methylase, high expression of UHRF1, high methylation level of nucleotide site of NNMT gene, and/or high methylation level of DNA CpG site of NNMT gene in the subject tumor, then administering the compound of the present invention to prevent and/or treat tumor.

### Composition or Preparation, Active Ingredient Combination, Medical Kit and Administration Method

Preferably, the composition of the present invention is pharmaceutical composition. The compositions of the present invention can comprise a pharmaceutically acceptable carrier.

The term “pharmaceutically acceptable carrier” refers to one or more compatible solid, semi-solid, liquid or gel fillers, which are suitable for use in human or animal and must have sufficient purity and sufficiently low toxicity. The “compatible” means each component and drug active ingredient in the pharmaceutical composition can be blended with each other without significantly reducing the efficacy.

It should be understood that the pharmaceutically acceptable carrier is not particularly limited in the present invention, the carrier can be selected from materials commonly used in the art, or can be obtained by a conventional method, or is commercially available. Some examples of pharmaceutically acceptable carriers are cellulose and its derivatives, polyols (e.g. propylene glycol, glycerin, mannitol, sorbitol, etc.), emulsifier (e.g. Tween), wetting agent (e.g. sodium lauryl sulfate), buffer agent, pH regulator, stabilizer, antioxidant, preservative, bacteriostatic agent, pyrogen-free water, etc.

In a preferred embodiment of the present invention, the dosage form of the composition or preparation is a solid preparation, liquid preparation or semi-solid preparation.

In a preferred embodiment of the present invention, the dosage form of the composition or preparation is oral preparation, external preparation or injection preparation Typically, the dosage form of the composition or preparation is tablet, injection, infusion, paste, gel, solution, microsphere or film.

The pharmaceutical preparation should be matched with the mode of administration. The pharmaceutical preparation of the present invention can also be given together with other synergistic therapeutic drugs before, during or after the administration. When the pharmaceutical composition or preparation is administrated, a safe and effective amount of the drug is administered to a subject in need (e.g. human or non-human mammal). The safe and effective amount is usually at least about 10 μg/kg·bw, and does not exceed about 8 μg/kg·bw in most case, preferably, the dose is about 10 μg/kg·bw to 1 mg/kg·bw. Of course, the specific dose should also take into account the route of administration, the patient's health and other factors, which are within the skill range of skilled doctors.

The main advantages of the present invention comprise:

The present has developed a compound, the compound has excellent precision treatment effect on tumor with low expression, no expression, low activity or no activity of mitochondria permeability transition pore, low expression, no expression, low activity or no activity of peptidyl-prolyl cis-trans isomerase F, low or no expression of NNMT gene, high expression of DNA methylase, high expression of UHRF1, high methylation level of nucleotide site of NNMT gene, and/or high methylation level of DNA CpG site of NNMT gene. The tumor with low expression, no expression, low activity or no activity of mitochondria permeability transition pore, low expression, no expression, low activity or no activity of peptidyl-prolyl cis-trans isomerase F, low or no expression of NNMT gene, high expression of DNA methylase, high expression of UHRF1, high methylation level of nucleotide site of NNMT gene, and/or high methylation level of DNA CpG site of NNMT gene is highly sensitive to the compound of present invention, ie, the compound of present invention has more remarkable and excellent treatment effect on tumor with low expression, no expression, low activity or no activity of mitochondria permeability transition pore, low expression, no expression, low activity or no activity of peptidyl-prolyl cis-trans isomerase F, low or no expression of NNMT gene, high expression of DNA methylase, high expression of UHRF1, high methylation level of nucleotide site of NNMT gene, and/or high methylation level of DNA CpG site of NNMT gene. Therefore, the compound of present invention can realize precise treatment on tumor with low expression, no expression, low activity or no activity of mitochondria permeability transition pore, low expression, no expression, low activity or no activity of peptidyl-prolyl cis-trans isomerase F, low or no expression of NNMT gene, high expression of DNA methylase, high expression of UHRF1, high methylation level of nucleotide site of NNMT gene, and/or high methylation level of DNA CpG site of NNMT gene, thus improving the treatment effect of the compound of present invention on tumor and avoiding administrating the compound of present invention to patient tumor insensitive to such anti-tumor drug. Therefore, the precision treatment of the compound of the present invention on tumor with low expression, no expression, low activity or no activity of mitochondria permeability transition pore, low expression, no expression, low activity or no activity of peptidyl-prolyl cis-trans isomerase F, low or no expression of NNMT gene, high expression of DNA methylase, high expression of UHRF1, high methylation level of nucleotide site of NNMT gene, and/or high methylation level of DNA CpG site of NNMT gene has advantages such as more excellent prevention and treatment effect on tumors, low drug dose, and minimal side effects, which improve the precision prevention and treatment effect on tumor, reduce the side effects and improve patient compliance.

The present invention will be further illustrated below with reference to the specific examples. It should be understood that these examples are only to illustrate the invention but are not to limit the scope of the invention. The experimental methods with no specific conditions described in the following examples are generally performed under the conventional conditions, or according to the manufacturer's instructions. Unless indicated otherwise, parts and percentage are calculated by weight.

### EXAMPLE

Mitochondria permeability transition pore was abbreviated as mPTP.

Peptidyl-prolyl cis-trans isomerase F was abbreviated as PPIF.

DNMT3a refers to DNA methyltransferase 3a, NCBI entrez gene: 1788; Uniprotkb/Swiss-port: Q9Y6K1.

DNMT3b refers to DNA methyltransferase 3b, NCBI entrez gene: 1789; Uniprotkb/Swiss-port: Q9UBC3.

DNMT1 refers to DNA methyltransferase 1, NCBI entrez gene: 1786; Uniprotkb/Swiss-port: P26358.

UHRF1 refers to ubiquitin-like with PHD and ring finger domain 1, NCBI entrez gene: 29128; Uniprotkb/Swiss-port:Q96T88.

NNMT refers to Nicotinamide N-Methyltransferase.

The nucleotide sequence of the promoter region of NNMT gene was as shown in SEQ ID NO: 1.

The nucleotide sites from 1050 bp before the transcription start site to 499 bp after the transcription start site in NNMT gene was sites 951-2500 of nucleotide sequence as shown in SEQ ID NO: 1.

The nucleotide sites from 1050 bp to 193 bp before the transcription start site in NNMT gene was sites 951-1808 of nucleotide sequence as shown in SEQ ID NO: 1.

The nucleotide sites from 840 bp to 469 bp before the transcription start site in NNMT gene was sites 1161-1532 of nucleotide sequence as shown in SEQ ID NO: 1.

### Example 1 Synthesis of Compound SJ0001

The structure of compound SJ0001 was as follows:

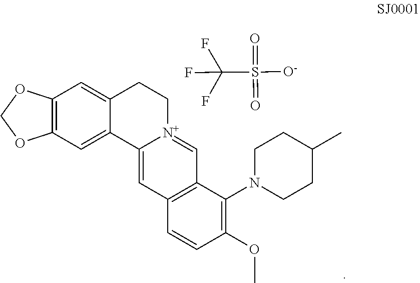 [SMILES: COc1ccc2cc3[n+](cc2c1N1CCC(C)CC1)CCc1cc2c(cc1-3)OCO2 ; O=S(=O)([O-])C(F)(F)F]

### Synthesis Method of Compound SJ0001

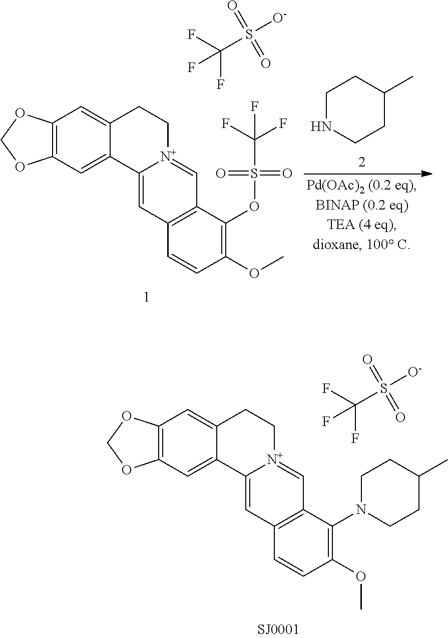

Compound 1 (300 mg, 0.49 mmol, 1.0 eq) and compound 2 (194 mg, 1.96 mmol, 4.0 eq) were dissolved in dioxane (5 mL), triethylamine (198 mg, 1.96 mmol, 4.0 eq), palladium acetate (22 mg, 0.098 mmol, 0.2 eq) and 1,1′-binaphthalene-2,2′-biphenylphosphine (61 mg, 0.098 mmol, 0.2 eq) were added. The mixture was stirred at 100° C. for 16 h under N₂, and the reaction was detected to be performed completely using LCMS. The reaction mixture was rotary dried, the crude product was purified by fast chromatography (dichloromethane/methanol=100/1 to 50/1) to afford compound SJ0001.

MS-ESI: calculated [M]⁺: 403.20, Found: 403.20.

¹H NMR (400 MHz, DMSO-d6): δ 9.62 (s, 1H), 8.87 (s, 1H), 8.10-7.99 (m, 2H), 7.76 (s, 1H), 7.05 (s, 1H), 6.14 (s, 2H), 4.96-4.93 (m, 2H), 3.99 (s, 3H), 3.27-3.25 (m, 2H), 3.20-3.16 (m, 2H), 2.90-2.87 (m, 2H), 1.68-1.51 (m, 5H), 1.01 (s, 3H).

### Example 2 Synthesis of Compound SJ0002

The structure of compound SJ0002 was as follows:

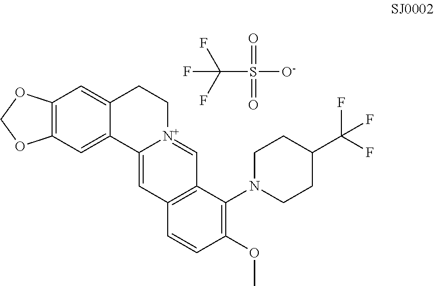 [SMILES: COc1ccc2cc3[n+](cc2c1N1CCC(C(F)(F)F)CC1)CCc1cc2c(cc1-3)OCO2 ; O=S(=O)([O-])C(F)(F)F]

### Synthesis Method of Compound SJ0002

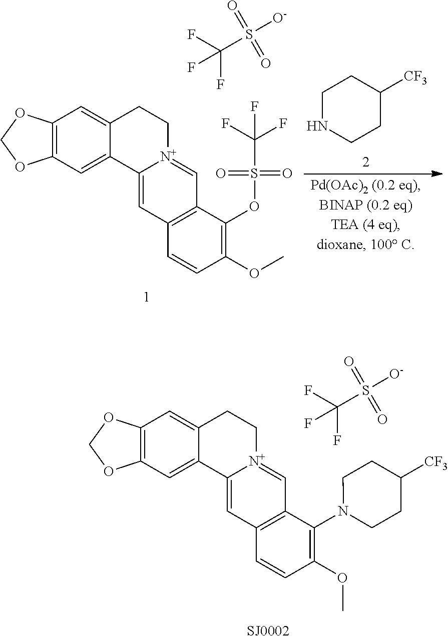 [SMILES: COc1ccc2cc3[n+](cc2c1OS(=O)(=O)C(F)(F)F)CCc1cc2c(cc1-3)OCO2 ; O=S(=O)([O-])C(F)(F)F ; FC(F)(F)C1CCNCC1 ; COc1ccc2cc3[n+](cc2c1N1CCC(C(F)(F)F)CC1)CCc1cc2c(cc1-3)OCO2 ; O=S(=O)([O-])C(F)(F)F]

Compound 1 (480 mg, 0.79 mmol, 1.0 eq) and compound 2 (484 mg, 3.16 mmol, 4.0 eq) were dissolved in dioxane (10 mL), triethylamine (319 mg, 3.16 mmol, 4.0 eq), palladium acetate (35 mg, 0.16 mmol, 0.2 eq) and 1,1′-binaphthalene-2,2′-biphenylphosphine (100 mg, 0.16 mmol, 0.2 eq) were added. The mixture was stirred at 100° C. for 16 h under N₂, and the reaction was detected to be performed completely using LCMS. The reaction mixture was rotary dried, the crude product was purified by fast chromatography (dichloromethane/methanol=100/1 to 50/1) to afford compound SJ0002.

MS-ESI calculated [M]⁺: 457.46, Found: 457.40.

¹H NMR (400 MHz, DMSO-d6): δ 9.70 (s, 1H), 8.89 (s, 1H), 8.12-8.06 (m, 2H), 7.76 (s, 1H), 7.07 (s, 1H), 6.14 (s, 2H), 4.98-4.95 (m, 2H), 4.00 (s, 3H), 3.29-3.27 (m, 2H), 3.19-3.16 (m, 2H), 3.06-2.99 (m, 2H), 1.97-1.84 (m, 5H).

### Example 3 Synthesis of Compound SJ0004

The structure of compound SJ0004 was as follows:

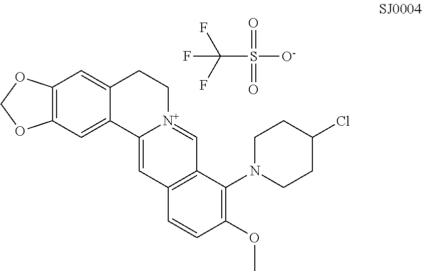 [SMILES: COc1ccc2cc3[n+](cc2c1N1CCC(Cl)CC1)CCc1cc2c(cc1-3)OCO2 ; O=S(=O)([O-])C(F)(F)F]

### Synthesis Method of Compound SJ0004

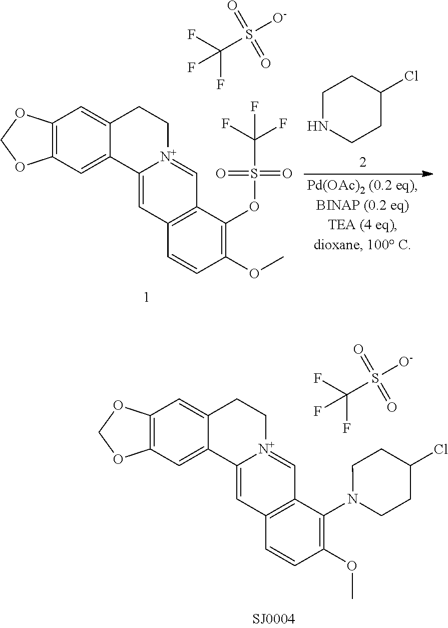 [SMILES: COc1ccc2cc3[n+](cc2c1OS(=O)(=O)C(F)(F)F)CCc1cc2c(cc1-3)OCO2 ; O=S(=O)([O-])C(F)(F)F ; ClC1CCNCC1 ; COc1ccc2cc3[n+](cc2c1N1CCC(Cl)CC1)CCc1cc2c(cc1-3)OCO2 ; O=S(=O)([O-])C(F)(F)F]

Compound 1 (480 mg, 0.79 mmol, 1.0 eq) and compound 2 (378 mg, 3.16 mmol, 4.0 eq) were dissolved in dioxane (10 mL), triethylamine (319 mg, 3.16 mmol, 4.0 eq), palladium acetate (35 mg, 0.16 mmol, 0.2 eq) and 1,1′-binaphthalene-2,2′-biphenylphosphine (100 mg, 0.16 mmol, 0.2 eq) were added. The mixture was stirred at 100° C. for 16 h under N₂, and the reaction was detected to be performed completely using LCMS. The reaction mixture was rotary dried, the crude product was purified by fast chromatography (dichloromethane/methanol=100/1 to 50/1) to afford compound SJ0004.

MS-ESI: calculated [M]+: 423.91, Found: 423.35.

¹H NMR (400 MHz, DMSO-d6): δ 9.73 (s, 1H), 8.88 (s, 1H), 8.12-8.02 (m, 2H), 7.76 (s, 1H), 7.07 (s, 1H), 6.14 (s, 2H), 4.97 (s, 2H), 4.00 (s, 3H), 3.29-3.27 (m, 3H), 3.19-3.17 (m, 2H), 2.99-2.79 (m, 2H), 2.15-1.82 (m, 4H).

### Example 4 Synthesis of Compound AB35528

The structure of compound AB35528 was as follows:

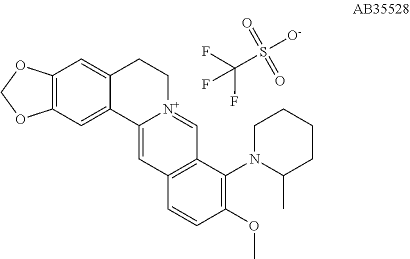 [SMILES: COc1ccc2cc3[n+](cc2c1N1CCCCC1C)CCc1cc2c(cc1-3)OCO2 ; O=S(=O)([O-])C(F)(F)F]

### Synthesis Method of Compound AB35528

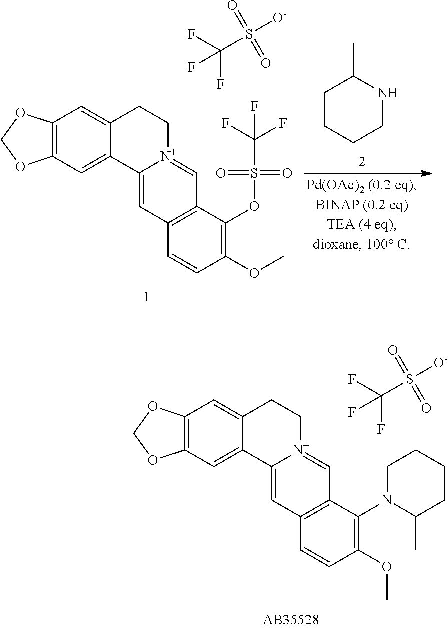

Compound 1 (800 mg, 1.32 mmol, 1.0 eq) and compound 2 (524 mg, 5.28 mmol, 4.0 eq) were dissolved in dioxane (10 mL), triethylamine (533 mg, 5.28 mmol, 4.0 eq), palladium acetate (59 mg, 0.264 mmol, 0.2 eq) and 1,1′-binaphthalene-2,2′-biphenylphosphine (164 mg, 0.264 mmol, 0.2 eq) were added. The mixture was stirred at 100° C. for 16 h under N₂, and the reaction was detected to be performed completely using LCMS. The reaction mixture was rotary dried, the crude product was purified by fast chromatography (dichloromethane/methanol=100/1 to 50/1) to afford compound AB35528.

MS-ESI: calculated [M]+: 403.20, Found: 403.20.

¹H NMR (400 MHz, DMSO-d6): δ 9.68 (s, 1H), 8.92 (s, 1H), 8.13-8.05 (m, 2H), 7.80 (s, 1H), 7.11 (s, 1H), 6.18 (s, 2H), 5.00-4.97 (m, 2H), 4.04 (s, 3H), 3.22-3.21 (m, 3H), 2.92-2.90-3.16 (m, 3H), 1.88-1.71 (m, 4H), 1.24-1.05 (m, 1H), 0.91 (s, 3H).

### Example 5 Synthesis of Compound AB35530

The structure of compound AB35530 was as follows:

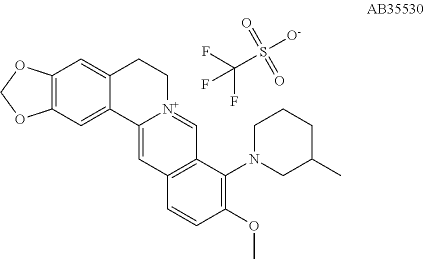 [SMILES: COc1ccc2cc3[n+](cc2c1N1CCCC(C)C1)CCc1cc2c(cc1-3)OCO2 ; O=S(=O)([O-])C(F)(F)F]

### Synthesis Method of Compound AB35530

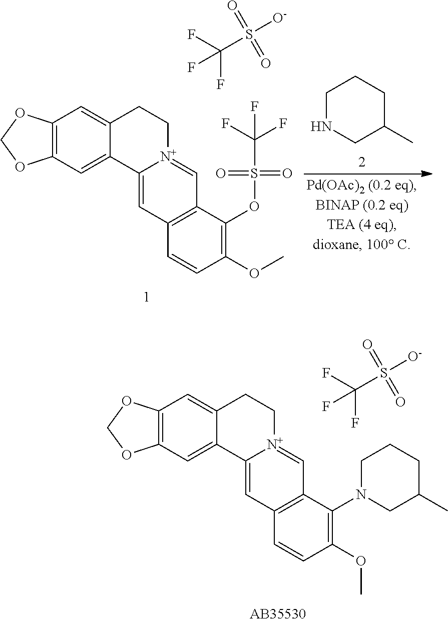

Compound 1 (500 mg, 0.83 mmol, 1.0 eq) and compound 2 (329 mg, 3.32 mmol, 4.0 eq) were dissolved in dioxane (10 mL), triethylamine (335 mg, 3.32 mmol, 4.0 eq), palladium acetate (37 mg, 0.166 mmol, 0.2 eq) and 1,1′-binaphthalene-2,2′-biphenylphosphine (103 mg, 0.166 mmol, 0.2 eq) were added. The mixture was stirred at 100° C. for 16 h under N₂, and the reaction was detected to be performed completely using LCMS. The reaction mixture was rotary dried, the crude product was purified by fast chromatography (dichloromethane/methanol=100/1 to 50/1) to afford compound AB35530.

MS-ESI: calculated [M]⁺: 403.20, Found: 403.20.

¹H NMR (400 MHz, DMSO-d6): δ 9.64 (s, 1H), 8.88 (s, 1H), 8.11-8.00 (m, 2H), 7.76 (s, 1H), 7.07 (s, 1H), 6.14 (s, 2H), 4.95-4.92 (m, 2H), 4.00 (s, 3H), 3.29-3.18 (m, 3H), 2.90 (m, 3H), 2.47-2.46 (m, 4H), 1.20 (s, 1H), 0.86 (s, 3H).

### Example 6 Synthesis of Compound AB35540

The structure of compound AB35540 was as follows:

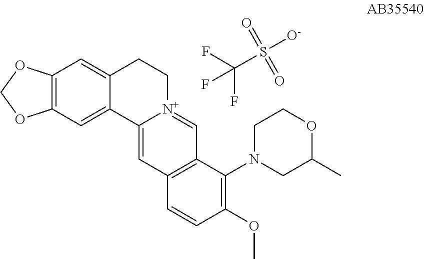 [SMILES: COc1ccc2cc3[n+](cc2c1N1CCOC(C)C1)CCc1cc2c(cc1-3)OCO2 ; O=S(=O)([O-])C(F)(F)F]

### Synthesis Method of Compound AB35540

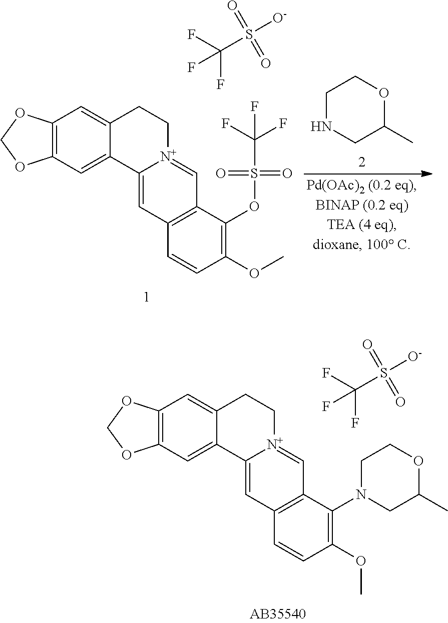

Compound 1 (500 mg, 0.83 mmol, 1.0 eq) and compound 2 (336 mg, 3.32 mmol, 4.0 eq) were dissolved in dioxane (10 mL), triethylamine (335 mg, 3.32 mmol, 4.0 eq), palladium acetate (37 mg, 0.166 mmol, 0.2 eq) and 1,1′-binaphthalene-2,2′-biphenylphosphine (103 mg, 0.166 mmol, 0.2 eq) were added. The mixture was stirred at 100° C. for 16 h under N₂, and the reaction was detected to be performed completely using LCMS. The reaction mixture was rotary dried, the crude product was purified by fast chromatography (dichloromethane/methanol=100/1 to 50/1) to afford compound AB35540.

MS-ESI: calculated [M]+: 405.18, Found: 405.20.

¹H NMR (400 MHz, DMSO-d6): δ 9.78 (s, 1H), 8.91 (s, 1H), 8.15-8.06 (m, 2H), 7.78 (s, 1H), 7.08 (s, 1H), 6.15 (s, 2H), 4.97-4.94 (m, 2H), 4.04 (s, 3H), 3.91-3.84 (m, 3H), 3.47-3.42 (m, 1H), 3.19-3.10 (m, 3H), 2.85-2.75 (m, 2H), 1.10 (d, J=4.0 Hz, 3H).

### Example 7 Synthesis of Compound AB35551

The structure of compound AB35551 was as follows:

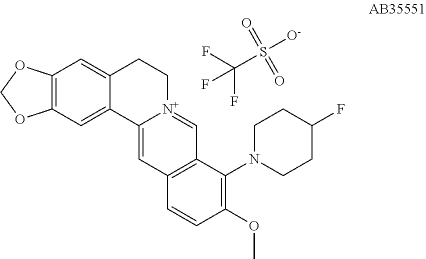 [SMILES: COc1ccc2cc3[n+](cc2c1N1CCC(F)CC1)CCc1cc2c(cc1-3)OCO2 ; O=S(=O)([O-])C(F)(F)F]

### Synthesis Method of Compound AB35551

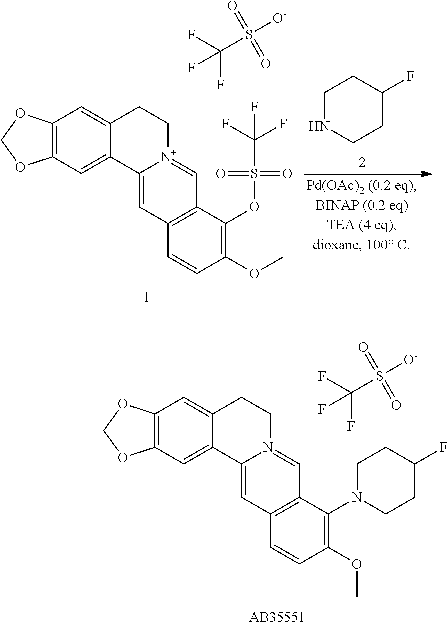 [SMILES: COc1ccc2cc3[n+](cc2c1OS(=O)(=O)C(F)(F)F)CCc1cc2c(cc1-3)OCO2 ; O=S(=O)([O-])C(F)(F)F ; FC1CCNCC1 ; COc1ccc2cc3[n+](cc2c1N1CCC(F)CC1)CCc1cc2c(cc1-3)OCO2 ; O=S(=O)([O-])C(F)(F)F]

Compound 1 (500 mg, 0.83 mmol, 1.0 eq) and compound 2 (329 mg, 3.32 mmol, 4.0 eq) were dissolved in dioxane (10 mL), triethylamine (335 mg, 3.32 mmol, 4.0 eq), palladium acetate (37 mg, 0.166 mmol, 0.2 eq) and 1,1′-binaphthalene-2,2′-biphenylphosphine (103 mg, 0.166 mmol, 0.2 eq) were added. The mixture was stirred at 100° C. for 16 h under N₂, and the reaction was detected to be performed completely using LCMS. The reaction mixture was rotary dried, the crude product was purified by fast chromatography (dichloromethane/methanol=100/1 to 50/1) to afford compound AB35551.

MS-ESI: calculated [M]+: 407.18, Found: 407.15.

¹H NMR (400 MHz, DMSO-d6): δ 9.72 (s, 1H), 8.88 (s, 1H), 8.11-8.02 (m, 2H), 7.76 (s, 1H), 7.06 (s, 1H), 6.14 (s, 2H), 4.97-4.94 (m, 2H), 4.00 (s, 3H), 3.63-3.51 (m, 1H), 3.27-3.23 (m, 2H), 3.20-3.17 (m, 2H), 3.04-2.75 (m, 2H), 3.21-1.91 (m, 4H).

### Example 8 Synthesis of Compound AB35555

The structure of compound AB35555 was as follows:

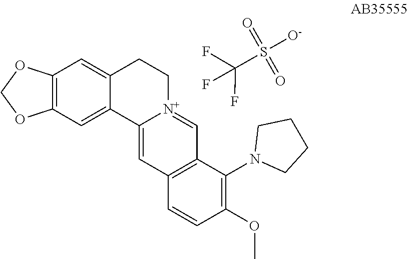 [SMILES: COc1ccc2cc3[n+](cc2c1N1CCCC1)CCc1cc2c(cc1-3)OCO2 ; O=S(=O)([O-])C(F)(F)F]

### Synthesis Method of Compound AB35555

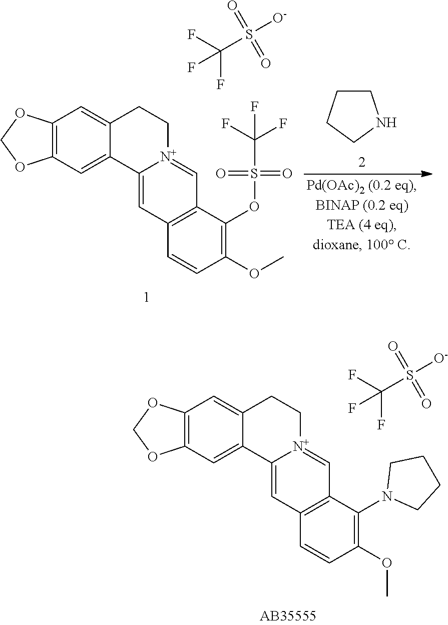 [SMILES: O=S(=O)([O-])C(F)(F)F ; COc1ccc2cc3[n+](cc2c1OS(=O)(=O)C(F)(F)F)CCc1cc2c(cc1-3)OCO2 ; C1CCNC1 ; COc1ccc2cc3[n+](cc2c1N1CCCC1)CCc1cc2c(cc1-3)OCO2 ; O=S(=O)([O-])C(F)(F)F]

Compound 1 (500 mg, 0.83 mmol, 1.0 eq) and compound 2 (236 mg, 3.32 mmol, 4.0 eq) were dissolved in dioxane (10 mL), triethylamine (335 mg, 3.32 mmol, 4.0 eq), palladium acetate (37 mg, 0.166 mmol, 0.2 eq) and 1,1′-binaphthalene-2,2′-biphenylphosphine (103 mg, 0.166 mmol, 0.2 eq) were added. The mixture was stirred at 100° C. for 16 h under N₂, and the reaction was detected to be performed completely using LCMS. The reaction mixture was rotary dried, the crude product was purified by fast chromatography (dichloromethane/methanol=100/1 to 50/1) to afford compound AB35555.

MS-ESI: calculated [M]+: 375.17, Found: 375.15.

¹H NMR (400 MHz, DMSO-d6): δ 9.78 (s, 1H), 8.90 (s, 1H), 8.16-8.02 (m, 2H), 7.79 (s, 1H), 7.09 (s, 1H), 6.17 (s, 2H), 4.96-4.93 (m, 2H), 4.04 (s, 3H), 3.35-3.33 (m, 4H), 3.22-3.19 (m, 2H), 2.05-2.02 (m, 4H).

### Example 9 Synthesis of Compound AB35556

The structure of compound AB35556 was as follows:

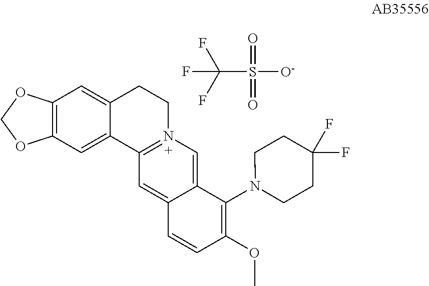

### Synthesis Method of Compound AB35556

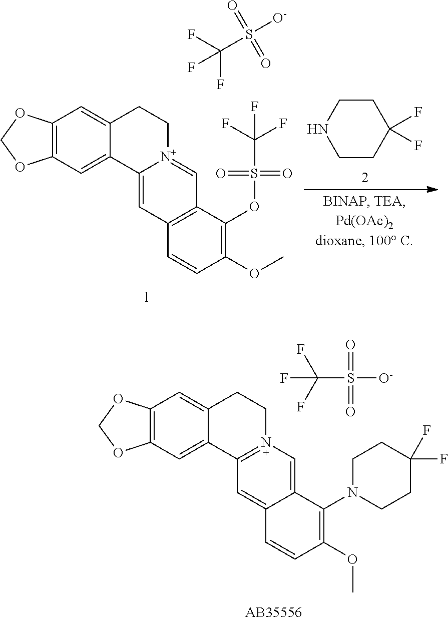

Compound 1 (500 mg, 0.83 mmol, 1.0 eq) and compound 2 (402 mg, 3.32 mmol, 4.0 eq) were dissolved in dioxane (10 mL), triethylamine (335 mg, 3.32 mmol, 4.0 eq), palladium acetate (37 mg, 0.166 mmol, 0.2 eq) and 1,1′-binaphthalene-2,2′-biphenylphosphine (103 mg, 0.166 mmol, 0.2 eq) were added. The mixture was stirred at 100° C. for 16 h under N₂, and the reaction was detected to be performed completely using LCMS. The reaction mixture was rotary dried, the crude product was purified by fast chromatography (dichloromethane/methanol=100/1 to 50/1) to afford compound AB35556.

MS-ESI: calculated [M]⁺: 425.17, Found: 425.15.

¹H NMR (400 MHz, DMSO-d6): δ 9.85 (s, 1H), 8.93 (s, 1H), 8.16-8.08 (m, 2H), 7.80 (s, 1H), 7.10 (s, 1H), 6.17 (s, 2H), 5.01-4.98 (m, 2H), 4.05 (s, 3H), 3.51-3.45 (m, 2H), 3.24-3.21 (m, 2H), 3.10-3.02 (m, 2H), 2.47-2.37 (m, 2H), 2.15-2.08 (m, 2H).

### Example 10 Synthesis of Compound AB35575

The structure of compound AB35575 was as follows:

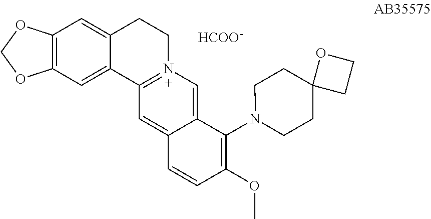

### Synthesis Method of Compound AB35575

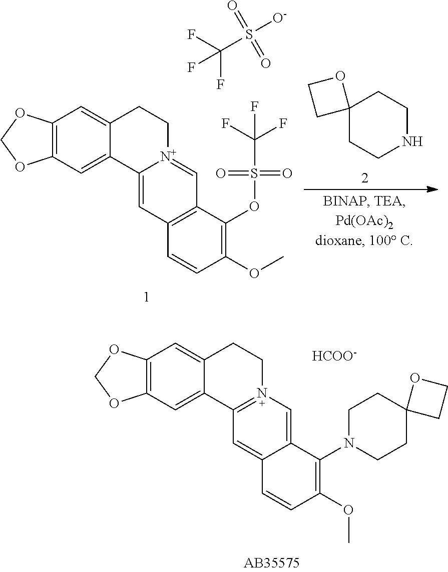

Compound 1 (500 mg, 0.83 mmol, 1.0 eq) and compound 2 (422 mg, 3.32 mmol, 4.0 eq) were dissolved in dioxane (10 mL), triethylamine (335 mg, 3.32 mmol, 4.0 eq), palladium acetate (38 mg, 0.17 mmol, 0.2 eq) and 1,1′-binaphthalene-2,2′-biphenylphosphine (106 mg, 0.17 mmol, 0.2 eq) were added. The mixture was stirred at 100° C. for 16 h under N₂, and the reaction was detected to be performed completely using LCMS. The reaction mixture was rotary dried, the crude product was purified by reversed preparation chromatography (acetonitrile/water+0.01% formic acid) to afford compound AB35575.

MS-ESI: calculated [M]⁺: 431.20, Found: 431.10.

¹H NMR (400 MHz, DMSO-d6): δ 9.69 (s, 1H), 8.88 (s, 1H), 8.10-8.00 (m, 2H), 7.76 (s, 1H), 7.06 (s, 1H), 6.14 (s, 2H), 4.96-4.93 (m, 2H), 4.45-4.42 (m, 2H), 3.98 (s, 3H), 3.69-3.40 (m, 2H), 3.19-3.16 (m, 2H), 3.01-2.74 (m, 2H), 2.45-2.42 (m, 2H), 2.07-2.04 (m, 4H).

### Example 11 Synthesis of Compound AB36413

The structure of compound AB36413 was as follows:

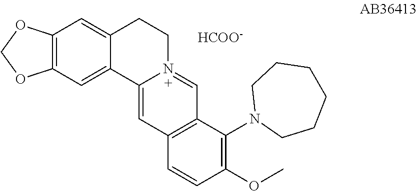

### Synthesis Method of Compound AB36413

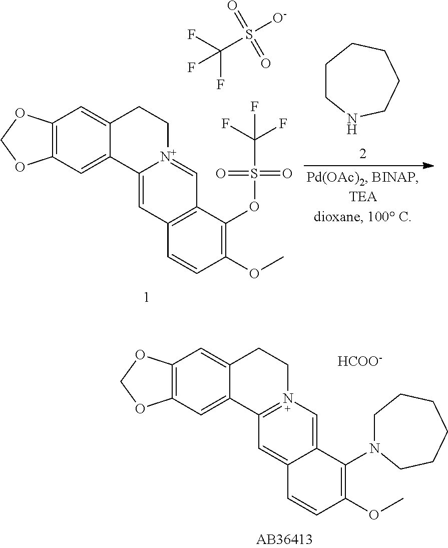

Compound 1 (700 mg, 1.16 mmol, 1.0 eq) and compound 2 (460 mg, 4.64 mmol, 4.0 eq) were dissolved in dioxane (10 mL), triethylamine (469 mg, 4.64 mmol, 4.0 eq), palladium acetate (52 mg, 0.23 mmol, 0.2 eq) and 1,1′-binaphthalene-2,2′-biphenylphosphine (143 mg, 0.23 mmol, 0.2 eq) were added. The mixture was stirred at 100° C. for 16 h under N₂, and the reaction was detected to be performed completely using LCMS. The reaction mixture was rotary dried, the crude product was purified by reversed preparation chromatography (acetonitrile/water+0.01% formic acid) to afford compound AB36413.

MS-ESI: calculated [M]⁺: 403.20, Found: 403.20.

¹H NMR (400 MHz, DMSO-d6): δ 9.63 (s, 1H), 8.91 (s, 1H), 8.17-8.05 (m, 2H), 7.79 (s, 1H), 7.10 (s, 1H), 6.17 (s, 2H), 4.98-4.95 (m, 2H), 4.04 (s, 3H), 3.24-3.18 (m, 7H), 1.79 (s, 8H).

### Example 12 Synthesis of Compound AB36492

The structure of compound AB36492 was as follows:

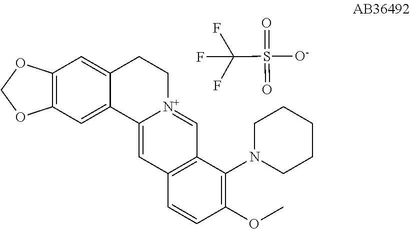 [SMILES: COc1ccc2cc3[n+](cc2c1N1CCCCC1)CCc1cc2c(cc1-3)OCO2 ; O=S(=O)([O-])C(F)(F)F]

### Synthesis Method of Compound AB36492

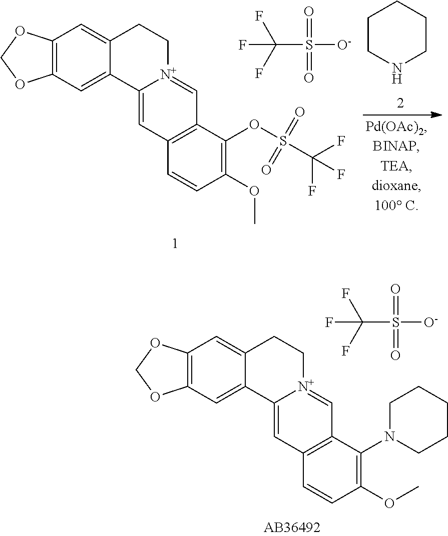 [SMILES: COc1ccc2cc3[n+](cc2c1OS(=O)(=O)C(F)(F)F)CCc1cc2c(cc1-3)OCO2 ; O=S(=O)([O-])C(F)(F)F ; C1CCNCC1 ; COc1ccc2cc3[n+](cc2c1N1CCCCC1)CCc1cc2c(cc1-3)OCO2 ; O=S(=O)([O-])C(F)(F)F]

Compound 1 (500 mg, 0.83 mmol, 1.0 eq) and compound 2 (283 mg, 3.32 mmol, 4.0 eq) were dissolved in dioxane (10 mL), triethylamine (335 mg, 3.32 mmol, 4.0 eq), palladium acetate (37 mg, 0.166 mmol, 0.2 eq) and 1,1′-binaphthalene-2,2′-biphenylphosphine (103 mg, 0.166 mmol, 0.2 eq) were added. The mixture was stirred at 100° C. for 16 h under N₂. The reaction mixture was rotary dried, the crude product was purified by fast chromatography (dichloromethane/methanol=100/1 to 50/1) to afford compound AB36492.

MS-ESI: calculated [M]⁺: 389.19, Found: 389.15.

¹H NMR (400 MHz, DMSO-d6): δ 9.67 (s, 1H), 8.91 (s, 1H), 8.13 (d, J=12.0 Hz, 1H), 8.04 (d, J=8.0 Hz, 1H), 7.79 (s, 1H), 7.10 (s, 1H), 6.17 (s, 2H), 4.99-4.96 (m, 2H), 4.03 (s, 3H), 3.30-3.24 (m, 2H), 3.23-3.19 (m, 2H), 3.10-2.92 (m, 2H), 1.87-1.43 (m, 6H).

### Example 13

Compound SJ0003 was prepared by conventional synthesis methods, the structure of compound SJ0003 was as follows:

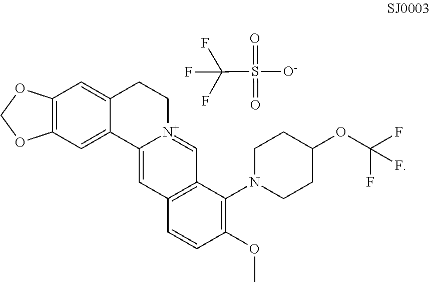 [SMILES: COc1ccc2cc3[n+](cc2c1N1CCC(OC(F)(F)F)CC1)CCc1cc2c(cc1-3)OCO2 ; O=S(=O)([O-])C(F)(F)F]

### Example 14

Compound SJ0005 was prepared by conventional synthesis methods, the structure of compound SJ0005 was as follows:

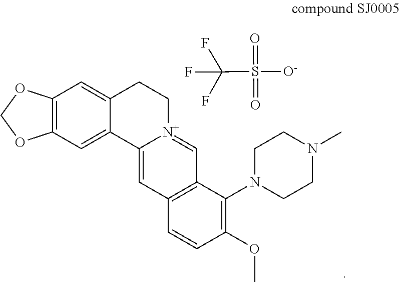 [SMILES: COc1ccc2cc3[n+](cc2c1N1CCN(C)CC1)CCc1cc2c(cc1-3)OCO2 ; O=S(=O)([O-])C(F)(F)F]

### Example 15

Compound SJ0006 was prepared by conventional synthesis methods, the structure of compound SJ0006 was as follows:

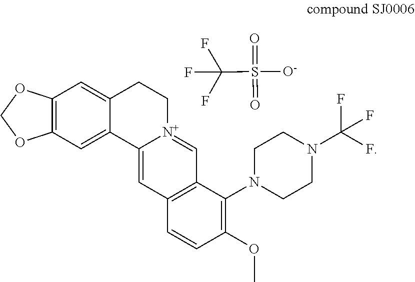 [SMILES: COc1ccc2cc3[n+](cc2c1N1CCN(C(F)(F)F)CC1)CCc1cc2c(cc1-3)OCO2 ; O=S(=O)([O-])C(F)(F)F]

### Example 16

Compound SJ0007 was prepared by conventional synthesis methods, the structure of compound SJ0007 was as follows:

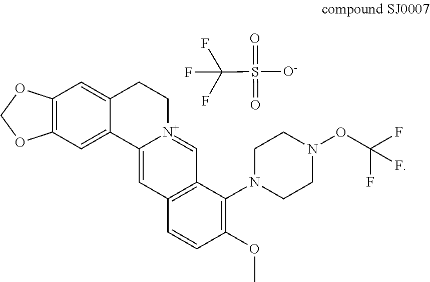 [SMILES: COc1ccc2cc3[n+](cc2c1N1CCN(OC(F)(F)F)CC1)CCc1cc2c(cc1-3)OCO2 ; O=S(=O)([O-])C(F)(F)F]

### Example 17

Compound SJ0008 was prepared by conventional synthesis methods, the structure of compound SJ0008 was as follows:

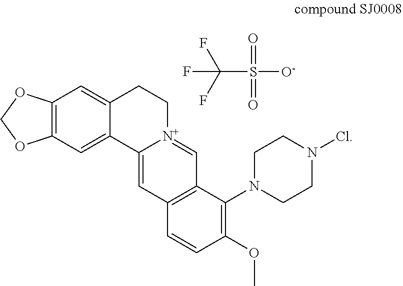 [SMILES: COc1ccc2cc3[n+](cc2c1N1CCN(Cl)CC1)CCc1cc2c(cc1-3)OCO2 ; O=S(=O)([O-])C(F)(F)F]

### Example 18

Compound SJ0009 was prepared by conventional synthesis methods, the structure of compound SJ0009 was as follows:

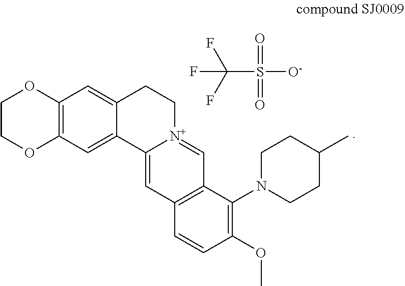 [SMILES: COc1ccc2cc3[n+](cc2c1N1CCC(C)CC1)CCc1cc2c(cc1-3)OCCO2 ; O=S(=O)([O-])C(F)(F)F]

### Example 19

Compound SJ0010 was prepared by conventional synthesis methods, the structure of compound SJ0010 was as follows:

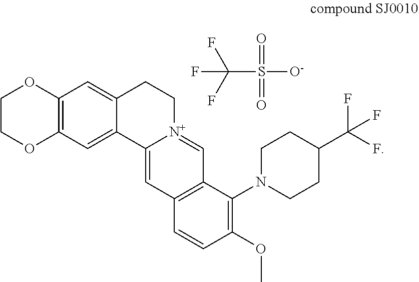 [SMILES: COc1ccc2cc3[n+](cc2c1N1CCC(C(F)(F)F)CC1)CCc1cc2c(cc1-3)OCCO2 ; O=S(=O)([O-])C(F)(F)F]

### Example 20

Compound SJ0011 was prepared by conventional synthesis methods, the structure of compound SJ0011 was as follows:

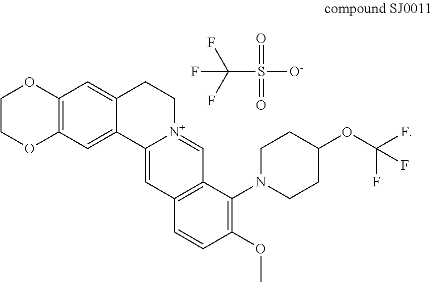 [SMILES: COc1ccc2cc3[n+](cc2c1N1CCC(OC(F)(F)F)CC1)CCc1cc2c(cc1-3)OCCO2 ; O=S(=O)([O-])C(F)(F)F]

### Example 21

Compound SJ0012 was prepared by conventional synthesis methods, the structure of compound SJ0012 was as follows:

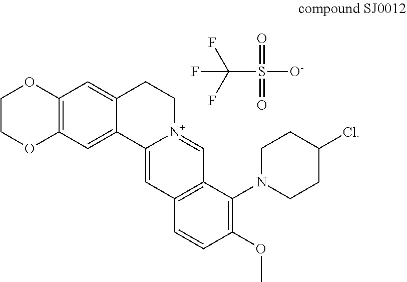 [SMILES: COc1ccc2cc3[n+](cc2c1N1CCC(Cl)CC1)CCc1cc2c(cc1-3)OCCO2 ; O=S(=O)([O-])C(F)(F)F]

### Example 22

Compound SJ0013 was prepared by conventional synthesis methods, the structure of compound SJ0013 was as follows:

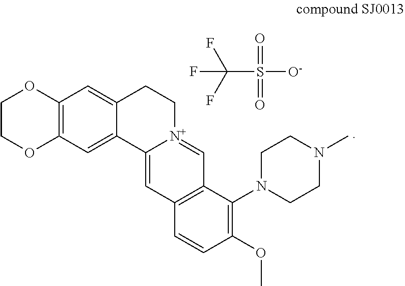 [SMILES: COc1ccc2cc3[n+](cc2c1N1CCN(C)CC1)CCc1cc2c(cc1-3)OCCO2 ; O=S(=O)([O-])C(F)(F)F]

### Example 23

Compound SJ0014 was prepared by conventional synthesis methods, the structure of compound SJ0014 was as follows:

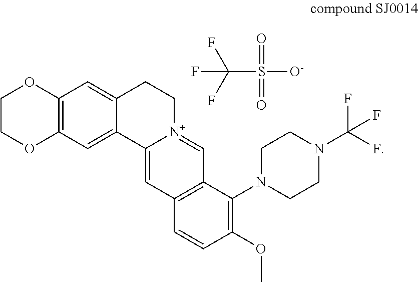 [SMILES: COc1ccc2cc3[n+](cc2c1N1CCN(C(F)(F)F)CC1)CCc1cc2c(cc1-3)OCCO2 ; O=S(=O)([O-])C(F)(F)F]

### Example 24

Compound SJ0015 was prepared by conventional synthesis methods, the structure of compound SJ0015 was as follows:

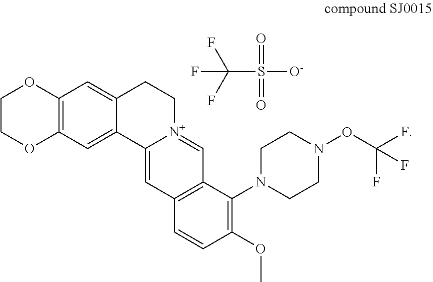 [SMILES: COc1ccc2cc3[n+](cc2c1N1CCN(OC(F)(F)F)CC1)CCc1cc2c(cc1-3)OCCO2 ; O=S(=O)([O-])C(F)(F)F]

### Example 25

Compound SJ0016 was prepared by conventional synthesis methods, the structure of compound SJ0016 was as follows:

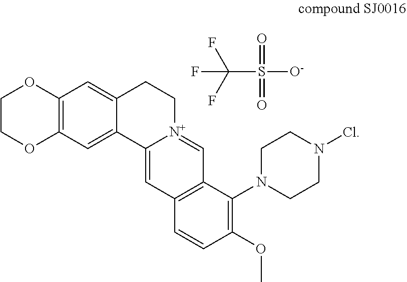 [SMILES: COc1ccc2cc3[n+](cc2c1N1CCN(Cl)CC1)CCc1cc2c(cc1-3)OCCO2 ; O=S(=O)([O-])C(F)(F)F]

### Example 26

### The Activity of Mitochondria Permeability Transition Pore in Related Cell was Investigated.

### Experimental Background:

The mitochondria permeability transition pore (mPTP) was a non-specific channel on the inner membrane of mitochondria, which could allow small molecules with molecular less than 1.5 KD to pass freely, and its activity was affected by peroxides (such as H₂O₂), pH and calcium ions in mitochondria. Some cells had active mitochondria permeability transition pore, while some cells had inactive mitochondria permeability transition pore. For cells with active mitochondria permeability transition pore, adding peroxide (such as H₂O₂) could increase the activity of mitochondria permeability transition pore, resulting in a decrease in mitochondria membrane potential. For cells with inactive mitochondria permeability transition pore, adding peroxide (such as H₂O₂) had no significant effect on the activity of the mitochondria permeability transition pore, and the mitochondria membrane potential had no significant change. Based on this principle, whether the mPTP was active or inactive in specific cell could be determined by measuring the change of potential difference of the mitochondria membrane under peroxide stimulation

### Experimental Method and Result:

Daoy cell (human medulloblastoma cell, ATCC no. HTB-186) were cultured in 10% fetal bovine serum-containing DMEM (+p/s), and then 1.5 μM Cyclosporin A (CSA, CSA could effectively inhibit the activity of mitochondria permeability transition pore) was added to the medium, and the cell cultured in medium without the addition of CsA was used as blank control. The mitochondria membrane potential difference was detected by Tetramethylrhodamine (TMRM), the high fluorescence intensity of TMRM represented the high membrane potential difference, the result was shown in Table 3.

TABLE 3
Relative TMRM signal intensity (%) in
Daoy cell after different treatments

| | |
|---|---|
| Cell | Daoy cell |

| | | | | |
|---|---|---|---|---|
| CsA | − | − | + | + |
| H₂O₂ | − | + | − | + |
| mean value | 100 | 82 | 125 | 121 |
| Standard deviation | 7 | 11 | 14 | 11 |
| Repeat times | 3 | 3 | 3 | 3 |
| P value | | <0.05 | <0.01 | |

Remarks: “+” represented existence, “−” represented none.

It could be seen from Table 3 that as for normal Daoy cell cultured in medium without the addition of CSA, the membrane potential was significantly down-regulated under the action of H₂O₂, indicating that the mPTP was active in Daoy cell. However, as for normal Daoy cell cultured in medium with the addition of CSA (CsA could inhibit the activity of mitochondria permeability transition pore (mPTP)), the membrane potential was not down-regulated under the action of H₂O₂, indicating that active mPTP of Daoy cell was inhibited by CsA and the mPTP of Daoy cell become inactive, H₂O₂ did not cause a decrease in membrane potential in this case.

Therefore, Table 3 showed that the mitochondria permeability transition pore was active in Daoy cell.

### Example 27

The inhibitory effect of the compounds prepared in the Examples of the present invention on the Daoy cell with normal mPTP activity, and the Daoy cell with low mPTP activity which was constructed by transfecting the Daoy cell with shRNA that target PPIF mRNA.

The peptidyl-prolyl cis-trans isomerase F was abbreviated as PPIF, the protein identification number was UniProtKB/Swiss Prot: P30405, and the gene identification number was NCBI Entrez Gene: 10105

1. Construction of Daoy Cell Having Inactive Mitochondria Permeability Transition Pore (mPTP)

### 1.1 Experimental Background:

The activity of mitochondria permeability transition pore (mPTP) was regulated by the PPIF protein. When PPIF protein was inhibited, mPTP activity was significantly decreased. PPIF protein activity was restricted by the expression level of PPIF protein in cell. The PPIF protein expression level in Daoy cell can be specifically decreased by transfecting the Daoy cell with shRNA that could specifically induce the degradation of PPIF mRNA, thereby constructing Daoy cell having inactive mitochondria permeability transition pore (mPTP) (abbreviated as mPTP-inactive Daoy cell).

### 1.2 Experiment Method and Result:

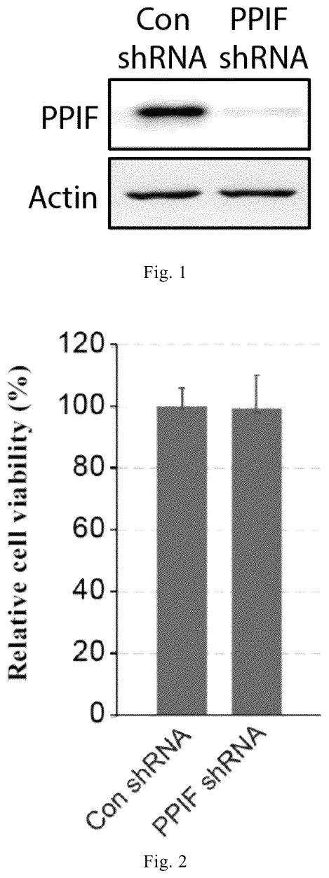

A viral vector carrying shRNA that could specifically induces the degradation of PPIF mRNA was obtained using cloning technology, the shRNA (the nucleotide sequence was GTTCTTCATCTGCACCATAAA (SEQ ID NO: 2)) carried by viral vector could specifically induce the degradation of PPIF mRNA, the Daoy cell was transfected with the viral vector carrying shRNA that could specifically induce the degradation of PPIF mRNA, and the Daoy cell transfected with empty virus vector without carrying shRNA that can specifically induce the degradation of PPIF mRNA was used as control. The expression level of PPIF protein in Daoy cell was detected using Western blot technique, and the result was shown in FIG. 1. The FIG. 1 showed the PPIF in the Daoy cell transfected with the shRNA that could specifically induce the degradation of PPIF (mPTP-regulated protein) mRNA was basically not expressed (i.e. PPIF shRNA in FIG. 1), while the PPIF in the Daoy cell not transfected with the shRNA that could specifically induce the degradation of PPIF mRNA was normally expressed (i.e. Con shRNA in FIG. 1, as the control). The mitochondria membrane potential difference was detected in the Daoy cell transfected with empty virus vector without carrying shRNA that could specifically induce the degradation of PPIF mRNA and the Daoy cell transfected with virus vector carrying shRNA that could specifically induce the degradation of PPIF mRNA using Tetramethylrhodamine (TMRM) according to the method described in Example 26 above, the high fluorescence intensity of TMRM represented the high membrane potential difference, the results were shown in Table 4 and Table 5.

TABLE 4
Relative TMRM signal intensity (%) in the Daoy cell transfected with
empty virus vector without carrying shRNA that could specificly
induce the degradation of PPIF mRNA after different treatments

| | The Daoy cell transfected with empty virus | | | |
|---|---|---|---|---|
| | vector without carrying shRNA that could specificly | | | |
| Cell | induce the degradation of PPIF mRNA | | | |
| CsA | − | − | + | + |
| H₂O₂ | − | + | − | + |
| mean value | 100 | 79 | 131 | 126 |
| Standard deviation | 6 | 7 | 10 | 8 |
| Repeat times | 3 | 3 | 3 | 3 |

TABLE 5
Relative TMRM signal intensity (%) in the Daoy cell transfected
with virus vector carrying shRNA that could specificly induce
the degradation of PPIF mRNA after different treatments

| | The Daoy cell transfected with virus vector carrying | | | |
|---|---|---|---|---|
| | shRNA that could specificly induce the degradation | | | |
| Cell | of PPIF mRNA | | | |
| CsA | − | − | + | + |
| H₂O₂ | − | + | − | + |
| mean value | 100 | 95 | 105 | 101 |
| Standard deviation | 5 | 6 | 8 | 11 |
| Repeat times | 3 | 3 | 3 | 3 |

The Table 4 and Table 5 showed the mitochondria permeability transition pore (mPTP) was active in the Daoy cell transfected with empty virus vector without carrying shRNA that could specifically induce the degradation of PPIF mRNA, the mitochondria permeability transition pore (mPTP) is inactive in the Daoy cell transfected with virus vector carrying shRNA that could specifically induce the degradation of PPIF mRNA, therefore, the Daoy cell having inactive mitochondria permeability transition pore (mPTP) was successfully constructed by transfecting the Daoy cell with shRNA that could specifically induce the degradation of PPIF (mPTP-regulated protein) mRNA.

The cell viability of the mPTP-inactive Daoy cell constructed by transfecting the Daoy cell with shRNA that could specifically induce the degradation of PPIF mRNA and the mPTP-active Daoy cell not transfected with shRNA that could specifically induce the degradation of PPIF mRNA was detected using the Promega CellTiter-Glo kit which reflected cell viability by measuring intracellular ATP content, the results was shown in FIG. 2, it could be seen from FIG. 2 that cell viability of mPTP-inactive Daoy cell constructed by transfecting the Daoy cell with shRNA that could specifically induce the degradation of PPIF mRNA and the mPTP-active Daoy cell not transfected with shRNA that could specifically induce the degradation of PPIF mRNA was almost the same, and the difference in cell viability was not statistically significant.

2. The Correlation Between the Inhibitory Effect of the Compounds Prepared in the Examples of the Present Invention on Tumor Cell and the Activity Level of mPTP was Investigated

### 2.1 Experimental Background:

The inhibitory effect (IC₅₀ value) of the compounds prepared in the Examples of the present invention on the mPTP-active Daoy cell not transfected with shRNA that could specifically induce the degradation of PPIF mRNA and the mPTP-inactive Daoy cell constructed by transfecting the Daoy cell with shRNA that could specifically induce the degradation of PPIF mRNA was detected using Promega CellTiter-Glo kit which reflected cell viability by directly measured intracellular ATP content.

### 2.2 Experiment Method and Result:

The mPTP-active Daoy cell not transfected with shRNA that could specifically induce the degradation of PPIF mRNA and the mPTP-inactive Daoy cell constructed by transfecting the Daoy cell with shRNA that could specifically induce the degradation of PPIF mRNA were cultured in 10% fetal bovine serum-containing DMEM medium (+p/s), the 50% inhibiting concentration (IC₅₀) of the compounds prepared in the Examples of present invention on the two types of cells was detected, and the experimental results were shown in Table 6:

TABLE 6
the inhibitory effect of the compounds prepared in
the Examples of present invention on the mPTP-active Daoy
cell and mPTP-inactive Daoy cell (IC₅₀, μM)

| | mPTP-active Daoy cell | mPTP-inactive Daoy cell |
|---|---|---|
| Compound | (IC₅₀, μM) | (IC₅₀, μM) |
| Compound SJ0001 | 1.35 | 0.52 |
| Compound SJ0002 | 5.13 | 1.56 |
| Compound SJ0004 | 7.45 | 2.41 |
| Compound SJ0009 | 2.06 | 0.47 |
| Compound SJ0010 | 6.33 | 2.09 |
| Compound SJ0012 | 6.96 | 2.08 |
| Compound AB35528 | 1.49 | 0.42 |
| Compound AB35530 | 2.43 | 0.55 |
| Compound AB35540 | 15.65 | 5.85 |
| Compound AB35551 | 5.82 | 1.97 |
| Compound AB35555 | 1.58 | 0.45 |
| Compound AB35556 | 7.31 | 3.14 |
| Compound AB35575 | 26.71 | 5.85 |
| Compound AB36413 | 1.26 | 0.32 |
| Compound AB36492 | 1.65 | 0.46 |

Noted:
IC₅₀ refered to 50% inhibiting concentration, ie, the concentration of the inhibitory compound when 50% inhibitory effect was achieved. The mPTP-active Daoy cell refered to the Daoy cell transfected with empty virus vector without carrying shRNA that can specificly induce the degradation of PPIF mRNA; the mPTP-inactive Daoy cell refered to the Daoy cell transfected with virus vector carrying shRNA that can specificly induce the degradation of PPIF mRNA.

As was shown in Table 6, the compounds prepared in the Examples of the present invention had significant inhibitory effect on mPTP-inactive Daoy cell, while the compounds prepared in the Examples of the present invention had weaker inhibitory effect on mPTP-active Daoy cell. Decreasing the mPTP activity in Daoy cell could significantly improve the inhibitory effect of the compounds prepared in the Examples of the present invention on tumor, therefore, the compounds prepared in the Examples of the present invention had more significant inhibitory effect on mPTP-inactive Daoy cell, the mPTP-inactive Daoy cell was highly sensitive to the compounds prepared in the Examples of the present invention. Therefore, the compounds prepared in the Examples of the present invention had excellent precision treatment effect on mPTP-inactive Daoy cell. As mentioned above, when PPIF protein was inhibited, mPTP activity was significantly decreased, inhibiting the expression level of PPIF protein could decrease the mPTP activity in Daoy cell. Therefore, the compounds prepared in the Examples of the present invention had more significant inhibitory effect on Daoy cell with low expression of PPIF protein, while the compounds prepared in the Examples of the present invention had weaker inhibitory effect on Daoy cell with normal expression of PPIF protein, indicating that decreasing the expression level of PPIF protein could significantly improve the inhibitory effect of the compounds prepared in the Examples of the present invention on tumor. Therefore, the compounds prepared in the Examples of the present invention had more significant inhibitory effect on Daoy cell with low expression of PPIF protein, i.e, the Daoy cell with low expression of PPIF protein was more sensitive to the compounds prepared in the Examples of the present invention compared with the Daoy cell with normal expression of PPIF protein. The compounds prepared in the Example of the present invention had excellent precision treatment on Daoy cell with low expression of PPIF protein.

### Example 28

The role of expression level of NNMT (Nicotinamide N-Methyltransferase) and DNMT1 (DNA methyltransferase 1) in the sensitivity of NCI-H82 cell (Human small cell lung cancer cell) to the compounds prepared in the Examples of the present invention was determined

### Experiment Method and Result:

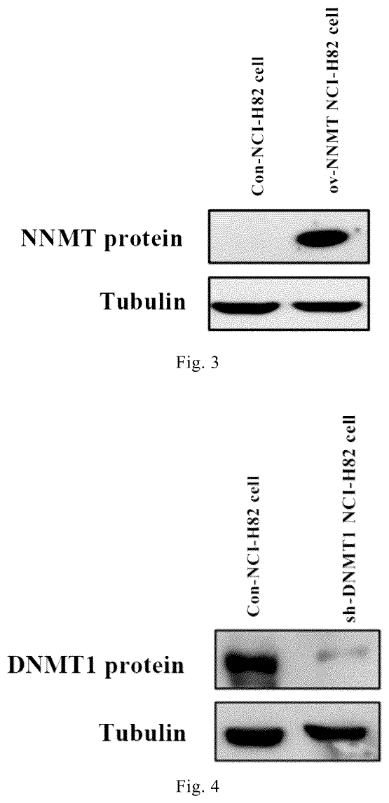

The expression of NNMT protein in NCI-H82 cell was overexpressed by inserting the NNMT gene into NCI-H82 cell using a viral vector, and the NNMT protein-overexpressing NCI-H82 cell (ov-NNMT NCI-H82 cell) was obtained. The expression of DNMT1 in NCI-H82 cell was knocked down by transfecting shRNA (the nucleotide sequence of shRNA was GATCCGGGATGAGTCCATCAAGGAAGATTCAAGAGATCTTCCTTGATGGACTCATCCTTTTTTG (SEQ ID No: 3)) using a viral vector, and the NCI-H82 cell with low expression of DNMT1 protein (sh-DNMT1 NCI-H82 cell) was obtained. The control NCI-H82 cell (Con-NCI-H82 cell) was obtained by transfecting NCI-H82 cell with empty virus vector without carrying NNMT gene and shRNA. The expressing content of NNMT protein and DNMT1 protein in the Con-NCI-H82 cell, the expressing content of NNMT protein in the ov-NNMT NCI-H82 cell, and the expressing content of DNMT1 protein in the sh-DNMT1 NCI-H82 cell were detected using Western Blot assay (as shown in FIG. 3 and FIG. 4), it could be seen from FIG. 3 and FIG. 4 that the NNMT protein was overexpressed in the ov-NNMT NCI-H82 cell and the expression of DNMT1 protein was knocked down (i.e, low expression) in the sh-DNMT1 NCI-H82 cell compared with the expression level of NNMT protein and DNMT1 protein in the Con-NCI-H82 cell.

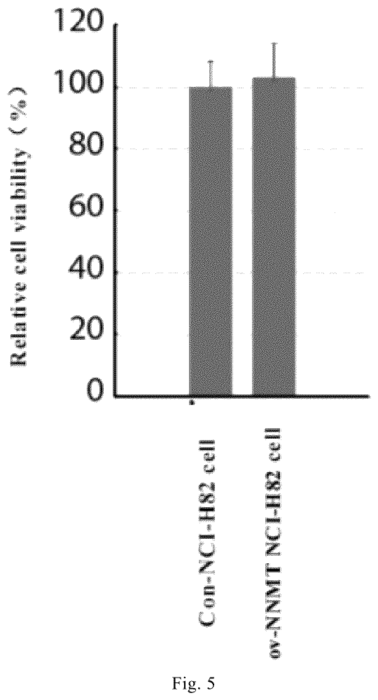

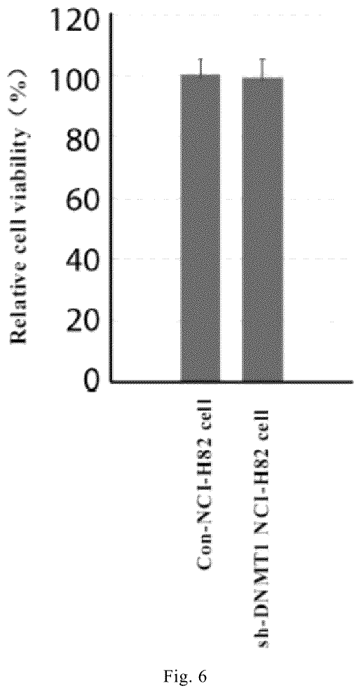

The cell viability of Con-NCI-H82 cell, ov-NNMT NCI-H82 cell and sh-DNMT1 NCI-H82 cell was detected using the Promega CellTiter-Glo kit which reflected cell viability by measuring intracellular ATP content (as shown in FIG. 5 and FIG. 6), it could be seen from FIG. 5 and FIG. 6 that the cell viability of Con-NCI-H82 cell, ov-NNMT NCI-H82 cell and sh-DNMT1 NCI-H82 cell was almost the same, and the difference in cell viability was not statistically significant.

The inhibitory effect (IC₅₀ value) of the compounds prepared in the Examples of the present invention on Con-NCI-H82 cell, ov-NNMT NCI-H82 cell and sh-DNMT1 NCI-H82 cell was detected using Promega CellTiter-Glo kit which directly measured intracellular ATP content, the result was shown in Table 7 below:

TABLE 7
the inhibitory effect of the compounds prepared in the
Examples of the present invention on Con-NCI-H82 cell, ov-
NNMT NCI-H82 cell and sh-DNMT1 NCI-H82 cell (IC₅₀, μM)

| | Con- | OV-NNMT | sh-DNMT1 |
|---|---|---|---|
| | NCI-H82 | NCI-H82 | NCI-H82 |
| Compound | (μM) | (μM) | (μM) |
| Compound SJ0001 | 0.14 | 0.58 | 1.12 |
| Compound SJ0002 | 0.32 | 0.88 | 2.03 |
| Compound SJ0004 | 0.74 | 1.94 | 2.96 |
| Compound SJ0009 | 0.18 | 0.55 | 1.33 |
| Compound SJ0010 | 0.45 | 0.89 | 2.54 |
| Compound SJ0012 | 0.97 | 2.35 | 3.88 |
| Compound AB35528 | 0.27 | 0.68 | 1.48 |
| Compound AB35530 | 0.32 | 0.72 | 1.76 |
| Compound AB35540 | 5.05 | 10.36 | 18.98 |
| Compound AB35551 | 1.57 | 3.48 | 5.96 |
| Compound AB35555 | 0.37 | 1.02 | 2.19 |
| Compound AB35556 | 2.57 | 5.85 | 9.86 |
| Compound AB35575 | 4.07 | 8.96 | 14.75 |
| Compound AB36413 | 0.29 | 0.85 | 1.58 |
| Compound AB36492 | 0.21 | 0.68 | 1.46 |

Note: IC₅₀ refered to 50% inhibiting concentration, ie, the concentration of the compound when 50% inhibitory effect was achieved. Con-NCI-H82 refered to the NCI-H82 cell transfected with empty virus vector without carrying NNMT gene and shRNA, which was used as control; ov-NNMT NCI-H82 refered to the NCI-H82 cell transfected with virus vector carrying NNMT gene, the NNMT protein was overexpressed in the ov-NNMT NCI-H82 cell; sh-DNMT1 NCI-H82 refered to the NCI-H82 cell transfected with virus vector carrying shRNA, the expression of DNMT1 was knocked down (i.e, low expression) in the sh-DNMT1 NCI-H82 cell.

As was shown in Table 7, the NNMT protein in NCI-1H82 cell was overexpressed and the expression of DNMT1 in NCI-1H82 cell was knocked down in the Example, the results further confirmed that the compounds prepared in the Examples of the present invention had significant inhibitory effect on tumor cell with low or no expression of NNMT gene and/or high expression of DNMT1, while the compounds prepared in the Examples of the present invention had weak inhibitory effect on tumor cells with high expression of NNMT gene and low expression of DNMT1. Decreasing the expression of NNMT gene and increasing the expression of DNMT1 in tumor could significantly improve the inhibitory effect of the compounds prepared in the Examples of the present invention on tumor on, the expression level of NNMT gene in tumor cell was significantly negative correlation with the sensitivity of tumor cell to the compounds prepared in the Examples of the present invention, while the expression level of DNMT1 in tumor cell was significantly positive correlation with the sensitivity of tumor cell to the compounds prepared in the Examples of the present invention. Therefore, the compounds prepared in the Examples of the present invention had more significant inhibitory effect on tumor cell with low or no expression of NNMT gene and/or high expression of DNMT1, the tumor cell with low or no expression of NNMT gene and/or high expression of DNMT1 were highly sensitive to the compounds prepared in the Examples of the present invention. Therefore, the compounds prepared in the Examples of the present invention has excellent precision treatment on the tumor cell with low or no expression of NNMT gene and/or high expression of DNMT1.

### Example 29

The methylation level of DNA in cell was maintained by DNA methylation enzymes DNMT3a, DNMT3b and DNMT1. The original methylation of DNA was performed with DNMT3a and DNMT3b, DNMT1 could replicate and maintain methylated DNA with the help of protein UHRF1 (ubiquitin-like with PHD and ring finger domain 1). The correlation between the expression of NNMT and the expression of DNMT1, UHRF1, DNMT3a and DNMT3b in tumor was determined in the Example.

### Experiment Method and Result:

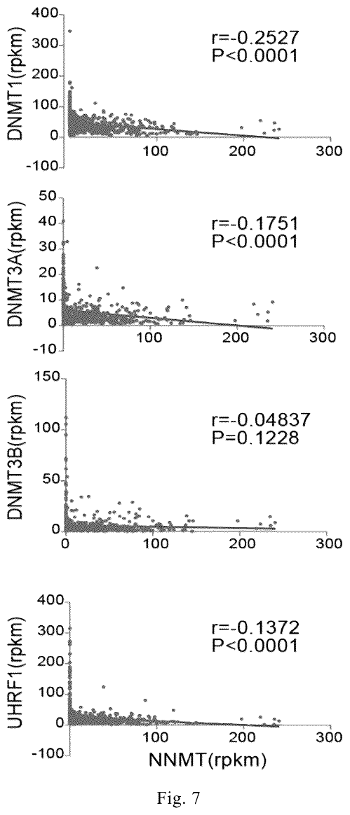

The expression of NNMT gene, DNMT1, UHRF1, DNMT3a and DNMT3b in various cells were obtained from a public database (Cancer Cell Line Encyclopedia, CCLE, 1019 cells in total). Then, the correlation between expression of NNMT and the expression of DNMT1, UHRF1, DNMT3a and DNMT3b in these cells was analyzed using bioinformatics, and the correlation between the expression level of NNMT gene and the expression level of DNMT1, UHRF1, DNMT3a and DNMT3b in each cell was analyzed, the experiment result was shown in FIG. 7.

The FIG. 7 showed the expression of NNMT was negatively correlated with the expression of DNA methylase (NMT3a, DNMT3b and DNMT1) and UHRF1 in each cell. Therefore, the tumor with high expression of DNA methylase (NMT3a, DNMT3b and DNMT1) and UHRF1 was highly sensitive to the compounds prepared in the Examples of the present invention, the compounds prepared in the Examples of the present invention had excellent precision treatment on the tumor with high expression of DNA methylase (NMT3a, DNMT3b and DNMT1) and UHRF1.

### Example 30

The inhibitory effect of the compounds prepared in the Examples of the present invention on various tumor cell lines was detected using cell viability assay reagent.

### Experimental Background:

Cell viability was detected using the Promega CellTiter-Glo kit, the cell viability was determined by directly measuring intracellular ATP content. In the experiment, the IC₅₀ value of the compounds prepared in the Examples of the present invention on various tumor cell lines was detected.

### Experimental Method and Result:

Each tumor cell was cultured in relevant medium. After cell passage, the gradient diluted compounds prepared in the Examples of the present invention was added. After 3 days of culture, the relevant IC₅₀ (50% inhibiting concentration) was measured. The name, source and culture conditions of each tumor cell line were as follows:

Cell line NCI-H82 (ATCC, No. HTB-175) was cultured in 10% fetal bovine serum-containing RPMI1640 medium (+P/S).

Cell line G-401 (ATCC, No. CRL-1441) was cultured in 10% fetal bovine serum-containing McCoy's 5a medium (+P/S).

Cell line MDA-MB-453 (ATCC, No. HTB-131) was cultured in 10% fetal bovine serum-containing Leibovitz's L-15 medium (+P/S).

Cell line SW48 (ATCC, No. CCL-231) was cultured in 10% fetal bovine serum-containing Leibovitz's L-15 medium (+P/S).

Cell line CFPAC-1 (ATCC, No. CRL-1918) was cultured in 10% fetal bovine serum-containing IMDM medium (+P/S).

Cell line 786-O (ATCC, No. CRL-1932) was cultured in 10% fetal bovine serum-containing RPMI1640 medium (+P/S).

Cell line GB-1 (JRCB, No. IFO 50489) was cultured in 10% fetal bovine serum-containing DMEM medium (+P/S).

Cell line SF-126 (JRCB, No. IFO 50286) was cultured in 10% fetal bovine serum-containing EMEM medium (+P/S).

The experimental result showed the sensitivity of different tumor cells to the compounds prepared in the Examples of the present invention, NCI-H82 (human small cell lung cancer cell), G-401 (human renal carcinoma Wilms cell), MDA-MB-453 (breast cancer cell) and SW48 (human colon adenocarcinoma cell) were sensitive to the compounds prepared in the Examples of the present invention with low IC₅₀ value, while 786-O (clear cell renal cell adenocarcinoma cell), CFPAC-1 (human pancreatic cancer cell), GB-1 (human brain glioblastoma cell) and SF126 (human glioblastoma multiforme cell) were not sensitive to the compounds prepared in the Examples of the present invention with high IC₅₀ value. The compounds prepared in the Examples of the present invention had more significant inhibitory effect on NCI-H82, G-401, MDA-MB-453, and SW48 tumor cells than 786-O, CFPAC-1, GB-1, and SF-126 cells.

### Example 31

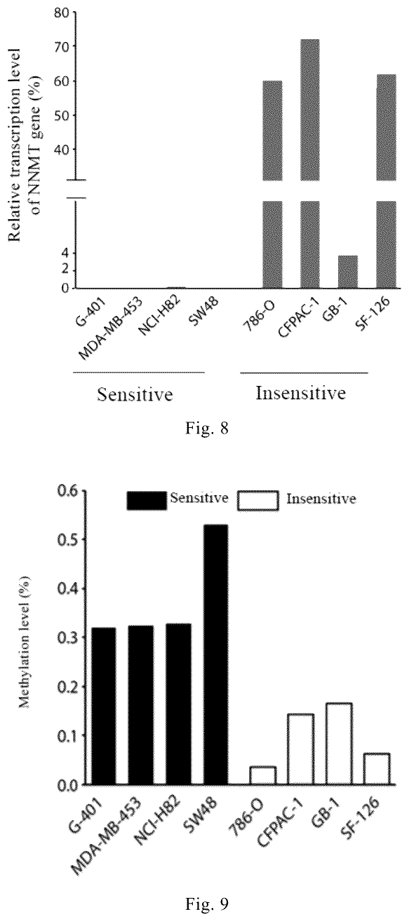

The mRNA transcription level of NNMT gene in four tumor cell lines sensitive to the compounds prepared in the Examples of the present invention and four tumor cell lines insensitive to the compounds prepared in the Examples of the present invention was measured using RT-qPCR gene expression analysis test, and the expression of NNMT gene in the tumor cell lines was measured respectively. The results were shown in FIG. 8.

As shown in FIG. 8, the mRNA transcription level of NNMT gene in four tumor cell lines (NCI-H82, G-401, MDA-MB-453 and SW48 cell) sensitive to the compounds prepared in the Examples of the present invention and four tumor cell lines (786-O, CFPAC-1, GB-1, and SF126 cell) insensitive to the compounds prepared in the Examples of the present invention was measured using RT-qPCR gene expression analysis test, the results showed the expression of NNMT gene was low in sensitive cell lines (NCI-H82, G-401, MDA-MB-453 and SW48 cell), and the expression of NNMT gene was high in insensitive cell lines (786-O, CFPAC-1, GB-1 and SF126 cell).

Therefore, the FIG. 8 showed that compared with tumor cell lines with high expression of NNMT gene, the inhibitory effect of the compounds prepared in the Examples of the present invention on tumor cell lines with low expression of NNMT gene was significantly enhanced, i.e, the expression of NNMT gene in tumor cell was negatively correlated with the sensitivity of tumor cell to the compounds prepared in the Examples of the present invention.

### Example 32

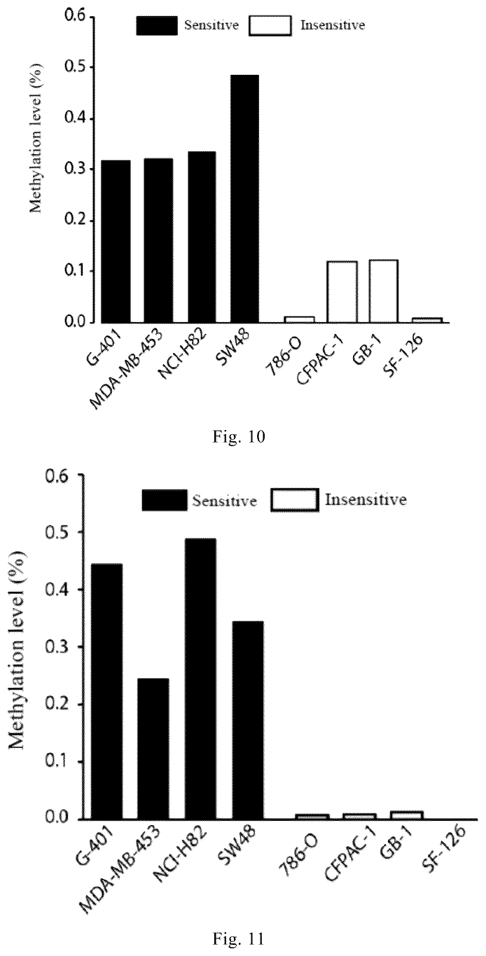

The promoter region of NNMT gene, the region from 1050 bp before the transcription start site to 499 bp after the transcription start site in NNMT gene and the region from 1050 bp to 193 bp before the transcription start site in NNMT gene were subjected to bisulfite sequencing to measure methylation level of DNA CpG site in four tumor cell lines (NCI-H82, G-401, MDA-MB-453 and SW48 cell) sensitive to the compounds prepared in the Examples of the present invention and four tumor cell lines (786-O, CFPAC-1, GB-1 and SF126 cell) insensitive to the compounds prepared in the Examples of the present invention. Firstly, genomic DNA was subjected to bisulfite, unmethylated cytosine was deamined to form uracil, and methylated cytosine could not be deamined, so the methylation sites could be determined by comparing the sequencing samples treated with bisulfite to the sequencing samples treated without bisulfite, and the result was shown in FIG. 9, FIG. 10, and FIG. 11.

As shown in FIG. 9 (the promoter region of NNMT gene), FIG. 10 (the region from 1050 bp before the transcription start site to 499 bp after the transcription start site in NNMT gene) and FIG. 11 (the region from 1050 bp to 193 bp before the transcription start site in NNMT gene), the compounds prepared in the Examples of the present invention had significantly stronger inhibitory effect on tumor cells with high methylation level of DNA CpG site in the promoter region of NNMT gene, the region from 1050 bp before the transcription start site to 499 bp after the transcription start site in NNMT gene and the region from 1050 bp to 193 bp before the transcription start site in NNMT gene, while the compounds prepared in the Examples of the present invention had significantly weaker inhibitory effect on tumor cells with low methylation level of DNA CpG site in the promoter region of NNMT gene, the region from 1050 bp before the transcription start site to 499 bp after the transcription start site in NNMT gene and the region from 1050 bp to 193 bp before the transcription start site in NNMT gene, indicating the methylation level of DNA CpG site in the promoter region of NNMT gene, the region from 1050 bp before the transcription start site to 499 bp after the transcription start site in NNMT gene and the region from 1050 bp to 193 bp before the transcription start site in NNMT gene was positively correlated with the sensitivity of tumor cell to the compounds prepared in the Examples of the present invention.

### Example 33

The methylation of specific DNA CpG sites from 840 bp (i.e., site 114165695 on human chromosome 11) to 469 bp (i.e., site 114166066 on human chromosome 11) before the transcription start site in NNMT gene in three tumor cell lines (NCI-H82, G-401 and SW48) sensitive to the compounds prepared in the Examples of the present invention and three tumor cell lines (786-O, CFPAC-1 and SF126) insensitive to the compounds prepared in the Examples of the present invention was studied.

Firstly, genomic DNA was subjected to bisulfite, unmethylated cytosine was deamined to form uracil, and methylated cytosine could not be deamined, so the methylation sites could be determined by comparing the sequencing samples treated with bisulfite to the sequencing samples treated without bisulfite, then PCR amplification and sequencing analysis were performed on the region using corresponding primers to measure the methylation level of CpG site in the DNA region.

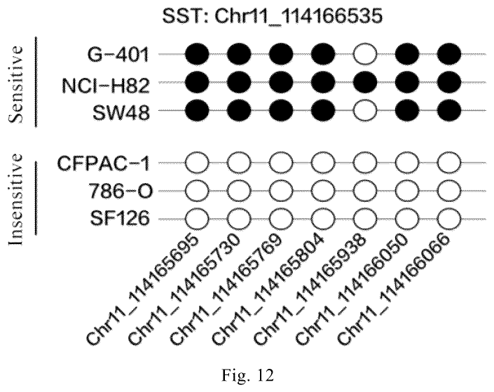

The study showed that almost all of the seven CpG sites (site 114165695, site 114165730, site 114165769, site 114165804, site 114165938, site 114166050 and site 114166066 on the human chromosome 11) were methylated in cell lines (NCI-H82, G-401 and SW48) sensitive to the compounds prepared in the Examples of the present invention, while none of the above seven CpG sites were methylated in cell lines ((786-O, CFPAC-1 and SF126) insensitive to the compounds prepared in the Examples of the present invention, the methylation of related sites was shown in FIG. 12.

The sites of the nucleotide sequence in SEQ ID NO: 1 corresponding to the site 114165695, site 114165730, site 114165769, site 114165804, site 114165938, site 114166050 and site 114166066 on the human chromosome 11 were as follows:

| | Corresponding to |
|---|---|
| | the sites of the |
| The site on the | nucleotide sequence |
| human chromosome 11 | in SEQ ID NO: 1 |
| site 114165695 | site 1161 |
| site 114165730 | site 1196 |
| site 114165769 | site 1235 |
| site 114165804 | site 1270 |
| site 114165938 | site 1404 |
| site 114166050 | site 1516 |
| site 114166066 | site 1532 |

All documents mentioned in the present invention are incorporated herein by reference, as if each document is individually cited for reference. It should be understood that those skilled in the art will be able to make various changes or modifications to the present invention after reading the teachings of the present invention, which also fall within the scope of the claims appended hereto.

1-14. (canceled)

15. A compound of formula I, or an optical isomer thereof, or a racemate thereof, or a solvate thereof, or a pharmaceutically acceptable salt thereof, or a deuterated compound thereof; 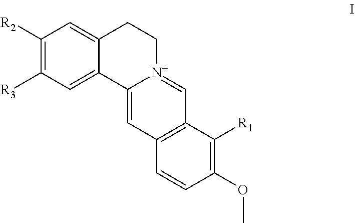
wherein,
R₁ is substituted or unsubstituted 3-12 membered heterocycloalkyl;
R₂ and R₃, together with the carbon atoms to which they are connected, form substituted or unsubstituted 3-12 membered heterocycloalkane ring;
each “substituted” means that one or more (preferably 1, 2, 3, 4, 5, 6, 7, or 8) hydrogen atoms on the ring or group are independently substituted by substituent selected from the group consisting of C1-C10 alkyl, C3-C8 cycloalkyl, C1-C10 haloalkyl, C3-C8 halocycloalkyl, C3-C8 cycloalkoxyl, C3-C8 cycloalkylthio, C3-C8 halocycloalkoxyl, C3-C8 halocycloalkylthio, halogen, nitro, —CN, hydroxyl, sulfhydryl, amino, C1-C4 carboxyl, C2-C8 ester group, C2-C4 amide group, C1-C10 alkoxyl, C1-C10 alkylthio, C1-C10 haloalkoxyl, C1-C10 haloalkylthio, C6-C12 aryl, 5-12 membered heteroaryl, 3-8 membered heterocycloalky;
the heterocyclic ring of the heterocycloalkyl, heteroaryl and heterocycloalkane ring has 1-4 (preferably 1, 2, 3 or 4) heteroatoms independently selected from the group consisting of N, O and S.

16. The compound of formula I, or an optical isomer thereof, or a racemate thereof, or a solvate thereof, or a pharmaceutically acceptable salt thereof, or a deuterated compound thereof of claim 15, wherein R₁ is substituted or unsubstituted 3 membered heterocycloalkyl, substituted or unsubstituted 4 membered heterocycloalkyl, substituted or unsubstituted 5 membered heterocycloalkyl, substituted or unsubstituted 6 membered heterocycloalkyl, substituted or unsubstituted 7 membered heterocycloalkyl, substituted or unsubstituted 8 membered heterocycloalkyl, substituted or unsubstituted 9 membered heterocycloalkyl, substituted or unsubstituted 10 membered heterocycloalkyl, substituted or unsubstituted 11 membered heterocycloalkyl, substituted or unsubstituted 12 membered heterocycloalkyl; and/or
R₂ and R₃, together with the carbon atoms to which they are connected, form substituted or unsubstituted 3 membered heterocycloalkane ring, substituted or unsubstituted 4 membered heterocycloalkane ring, substituted or unsubstituted 5 membered heterocycloalkane ring, substituted or unsubstituted 6 membered heterocycloalkane ring, substituted or unsubstituted 7 membered heterocycloalkane ring, substituted or unsubstituted 8 membered heterocycloalkane ring, substituted or unsubstituted 9 membered heterocycloalkane ring, substituted or unsubstituted 10 membered heterocycloalkane ring, substituted or unsubstituted 11 membered heterocycloalkane ring, substituted or unsubstituted 12 membered heterocycloalkane ring.

17. The compound of formula I, or an optical isomer thereof, or a racemate thereof, or a solvate thereof, or a pharmaceutically acceptable salt thereof, or a deuterated compound thereof of claim 15, wherein R₁ is substituted or unsubstituted piperazinyl, substituted or unsubstituted piperidinyl, substituted or unsubstituted morpholinyl, substituted or unsubstituted tetrahydropyrrolyl, substituted or unsubstituted piperidinospirooxetane group, substituted or unsubstituted hexamethyleneimine group; and/or
R₂ and R₃, together with the carbon atoms to which they are connected, form dioxole, dihydro-dioxine.

18. The compound of formula I, or an optical isomer thereof, or a racemate thereof, or a solvate thereof, or a pharmaceutically acceptable salt thereof, or a deuterated compound thereof of claim 15, wherein R₁ is 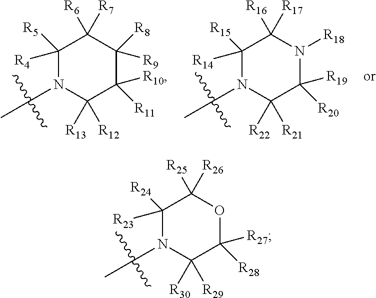
and/or
R₂ and R₃, together with the carbon atoms to which they are connected, form 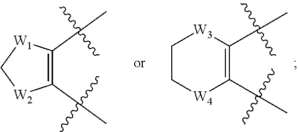
wherein, R₄, R₅, R₆, R₇, R₈, R₉, R₁₀, R₁₁, R₁₂ and R₁₃ are each independently hydrogen, C1-C10 alkyl, C1-C10 haloalkyl, C1-C10 haloalkoxyl, C1-C10 haloalkylthio, halogen;
R₁₄, R₁₅, R₁₆, R₁₇, R₁₈, R₁₉, R₂₀, R₂₁ and R₂₂ are each independently hydrogen, C1-C10 alkyl, C1-C10 haloalkyl, C1-C10 haloalkoxyl, C1-C10 haloalkylthio, halogen;
R₂₃, R₂₄, R₂₅, R₂₆, R₂₇, R₂₈, R₂₉ and R₃₀ are each independently hydrogen, C1-C10 alkyl;
W₁ and W₂ are each independently O or S;
W₃ and W₃ are each independently O or S.

19. The compound of formula I, or an optical isomer thereof, or a racemate thereof, or a solvate thereof, or a pharmaceutically acceptable salt thereof, or a deuterated compound thereof of claim 15, wherein R₁ is 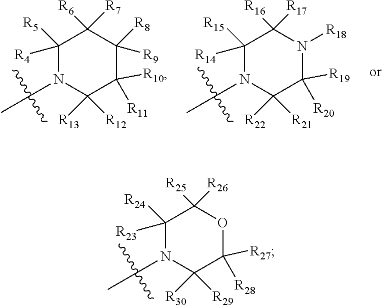
and/or
R₂ and R₃, together with the carbon atoms to which they are connected, form 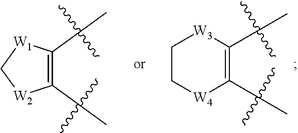
wherein, R₄, R₅, R₆, R₇, R₈, R₉, R₁₀, R₁₁, R₁₂ and R₁₃ are each independently hydrogen, C1-C4 alkyl, C1-C4 haloalkyl, C1-C4 haloalkoxyl, C1-C4 haloalkylthio, halogen;
R₁₄, R₁₅, R₁₆, R₁₇, R₁₈, R₁₉, R₂₀, R₂₁ and R₂₂ are each independently hydrogen, C1-C4 alkyl, C1-C4 haloalkyl, C1-C4 haloalkoxyl, C1-C4 haloalkylthio, halogen;
R₂₃, R₂₄, R₂₅, R₂₆, R₂₇, R₂₈, R₂₉ and R₃₀ are each independently hydrogen, C1-C10 alkyl;
W₁ and W₂ are each independently O or S;
W₃ and W₃ are each independently O or S.

20. The compound of formula I, or an optical isomer thereof, or a racemate thereof, or a solvate thereof, or a pharmaceutically acceptable salt thereof, or a deuterated compound thereof of claim 15, wherein R₁ is 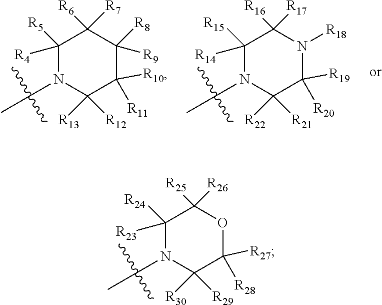
and/or
R₂ and R₃, together with the carbon atoms to which they are connected, form 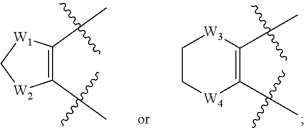
wherein, R₄, R₅, R₆, R₇, R₈, R₉, R₁₀, R₁₁, R₁₂ and R₁₃ are each independently hydrogen, methyl, trifluoromethyl, trifluoromethoxyl, halogen (e.g, fluorine, chlorine);
R₁₄, R₁₅, R₁₆, R₁₇, R₁₈, R₁₉, R₂₀, R₂₁ and R₂₂ are each independently hydrogen, methyl, trifluoromethyl, trifluoromethoxyl, halogen (e.g, chlorine);
R₂₃, R₂₄, R₂₅, R₂₆, R₂₇, R₂₈, R₂₉ and R₃₀ are each independently hydrogen, methyl;
W₁ and W₂ are each independently O or S;
W₃ and W₃ are each independently O or S.

21. The compound of formula I, or an optical isomer thereof, or a racemate thereof, or a solvate thereof, or a pharmaceutically acceptable salt thereof, or a deuterated compound thereof of claim 15, wherein the pharmaceutically acceptable salt of the compound of formula I comprises the salt formed by the compound of formula I and hydrochloric acid, mucic acid, D-glucuronic acid, hydrobromic acid, hydrofluoric acid, hydroiodic acid, sulfuric acid, nitric acid, phosphoric acid, formic acid, acetic acid, trifluoroacetic acid, propionic acid, oxalic acid, malonic acid, succinic acid, fumaric acid, maleic acid, lactic acid, malic acid, tartaric acid, citric acid, picric acid, methanesulfonic acid, benzenemethanesulfonic acid, benzenesulfonic acid, trifluoro methanesulfonic acid, aspartic acid or glutamic acid.

22. The compound of formula I, or an optical isomer thereof, or a racemate thereof, or a solvate thereof, or a pharmaceutically acceptable salt thereof, or a deuterated compound thereof of claim 15, wherein the compound is selected from the following group:

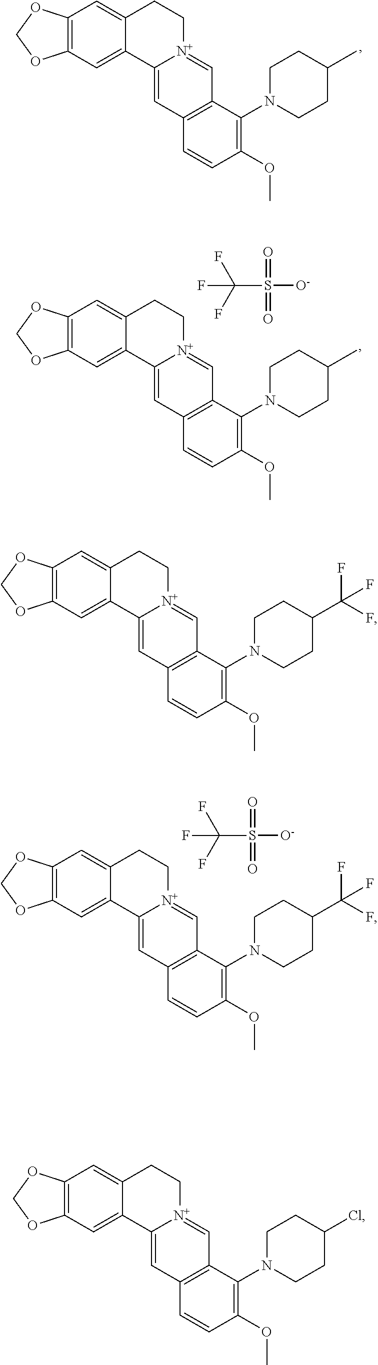

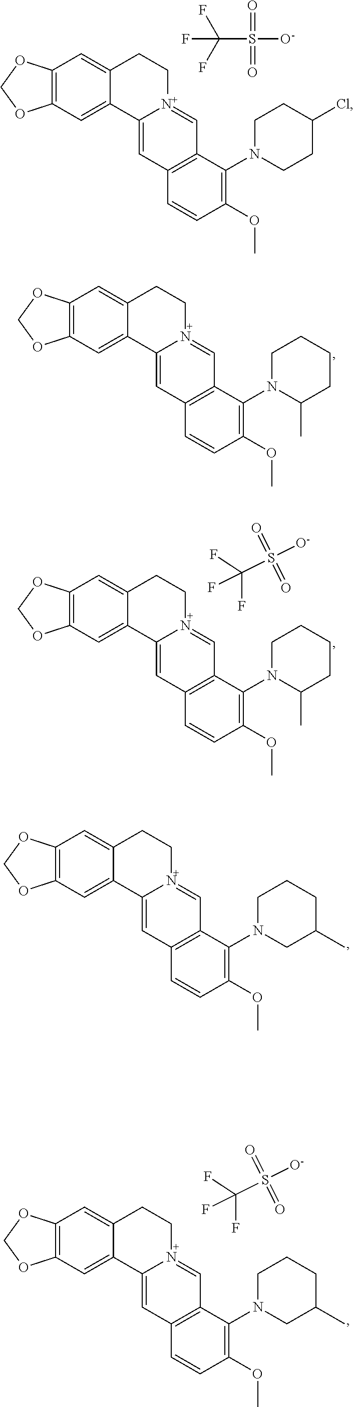

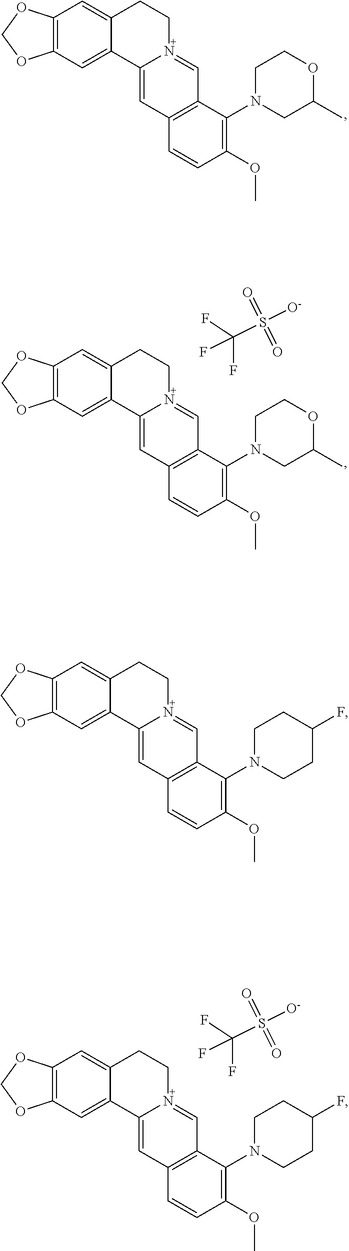

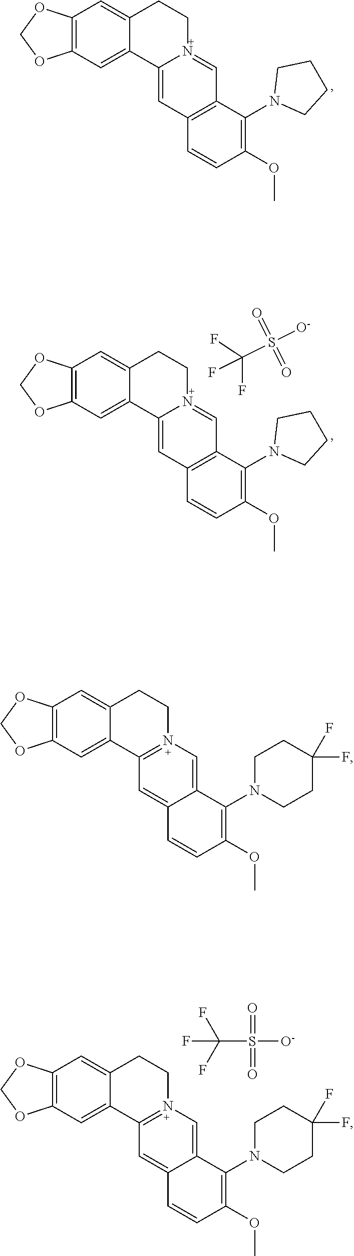

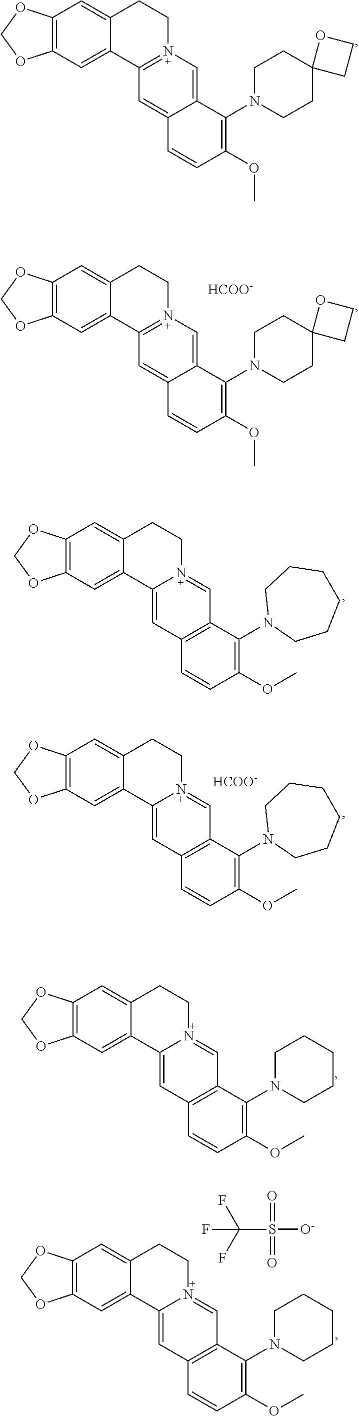

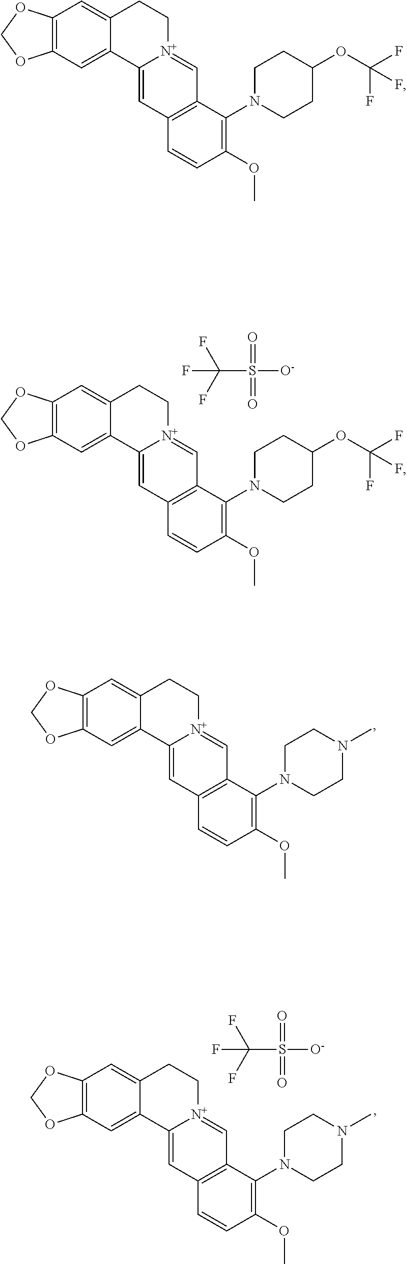 [SMILES: COc1ccc2cc3[n+](cc2c1N1CCC(OC(F)(F)F)CC1)CCc1cc2c(cc1-3)OCO2 ; COc1ccc2cc3[n+](cc2c1N1CCC(OC(F)(F)F)CC1)CCc1cc2c(cc1-3)OCO2 ; O=S(=O)([O-])C(F)(F)F ; COc1ccc2cc3[n+](cc2c1N1CCN(C)CC1)CCc1cc2c(cc1-3)OCO2 ; COc1ccc2cc3[n+](cc2c1N1CCN(C)CC1)CCc1cc2c(cc1-3)OCO2 ; O=S(=O)([O-])C(F)(F)F]

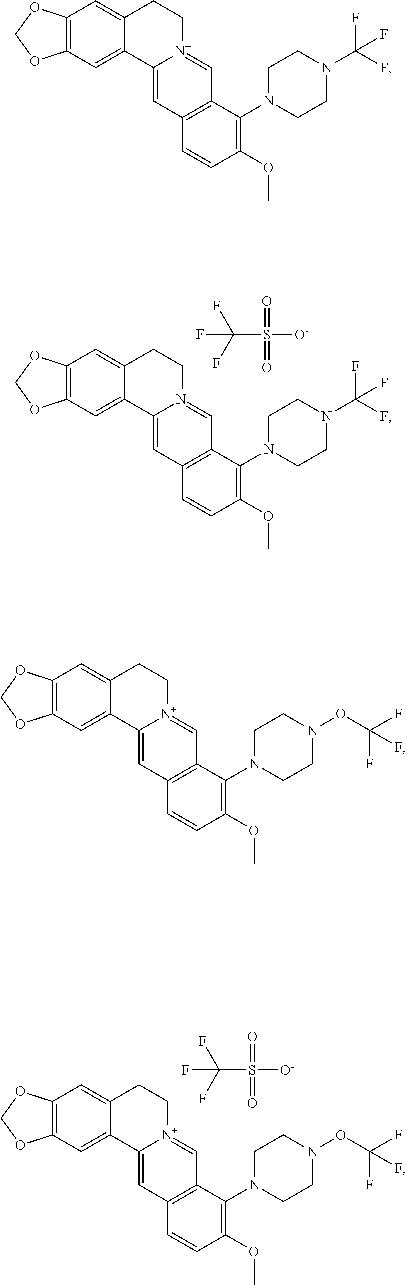 [SMILES: COc1ccc2cc3[n+](cc2c1N1CCN(C(F)(F)F)CC1)CCc1cc2c(cc1-3)OCO2 ; COc1ccc2cc3[n+](cc2c1N1CCN(C(F)(F)F)CC1)CCc1cc2c(cc1-3)OCO2 ; O=S(=O)([O-])C(F)(F)F ; COc1ccc2cc3[n+](cc2c1N1CCN(OC(F)(F)F)CC1)CCc1cc2c(cc1-3)OCO2 ; COc1ccc2cc3[n+](cc2c1N1CCN(OC(F)(F)F)CC1)CCc1cc2c(cc1-3)OCO2 ; O=S(=O)([O-])C(F)(F)F]

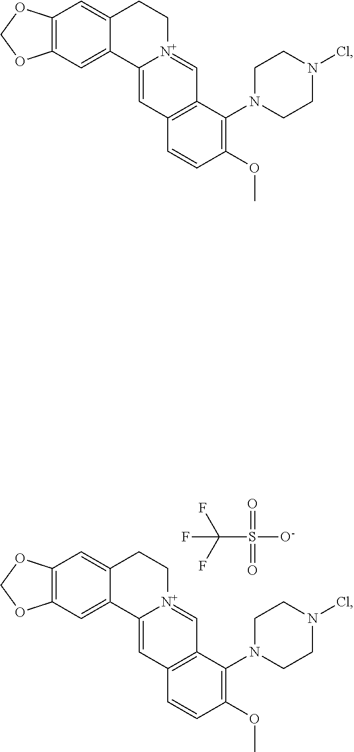 [SMILES: COc1ccc2cc3[n+](cc2c1N1CCN(Cl)CC1)CCc1cc2c(cc1-3)OCO2 ; COc1ccc2cc3[n+](cc2c1N1CCN(Cl)CC1)CCc1cc2c(cc1-3)OCO2 ; O=S(=O)([O-])C(F)(F)F]

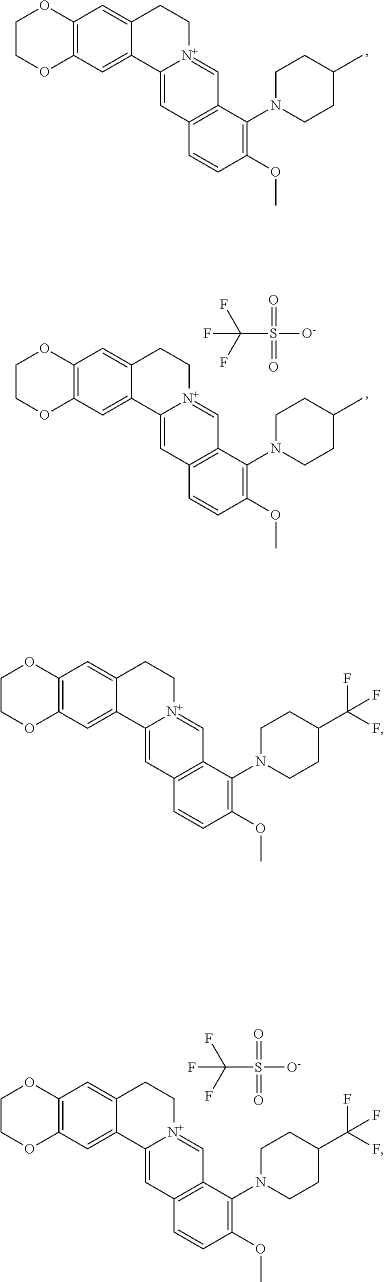 [SMILES: COc1ccc2cc3[n+](cc2c1N1CCC(C)CC1)CCc1cc2c(cc1-3)OCCO2 ; COc1ccc2cc3[n+](cc2c1N1CCC(C)CC1)CCc1cc2c(cc1-3)OCCO2 ; O=S(=O)([O-])C(F)(F)F ; COc1ccc2cc3[n+](cc2c1N1CCC(C(F)(F)F)CC1)CCc1cc2c(cc1-3)OCCO2 ; COc1ccc2cc3[n+](cc2c1N1CCC(C(F)(F)F)CC1)CCc1cc2c(cc1-3)OCCO2 ; O=S(=O)([O-])C(F)(F)F]

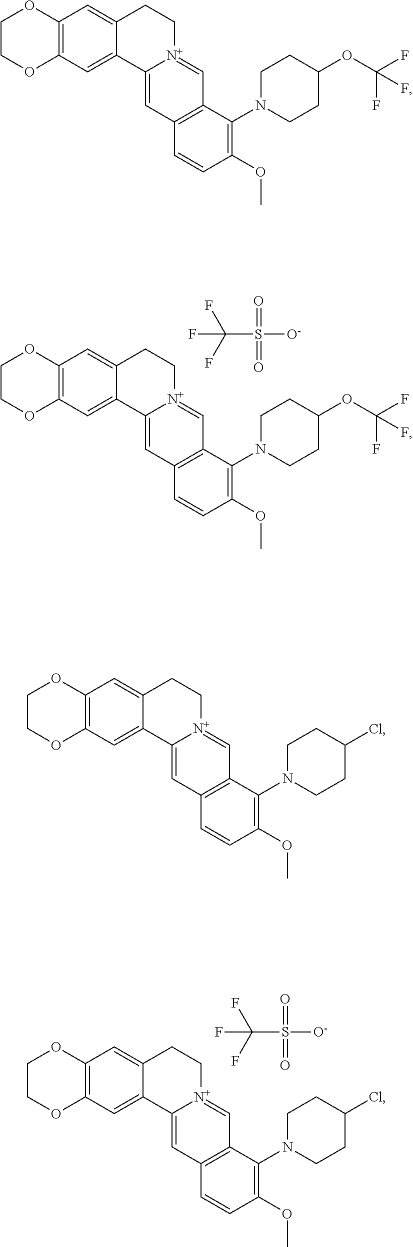 [SMILES: COc1ccc2cc3[n+](cc2c1N1CCC(OC(F)(F)F)CC1)CCc1cc2c(cc1-3)OCCO2 ; COc1ccc2cc3[n+](cc2c1N1CCC(OC(F)(F)F)CC1)CCc1cc2c(cc1-3)OCCO2 ; O=S(=O)([O-])C(F)(F)F ; COc1ccc2cc3[n+](cc2c1N1CCC(Cl)CC1)CCc1cc2c(cc1-3)OCCO2 ; COc1ccc2cc3[n+](cc2c1N1CCC(Cl)CC1)CCc1cc2c(cc1-3)OCCO2 ; O=S(=O)([O-])C(F)(F)F]

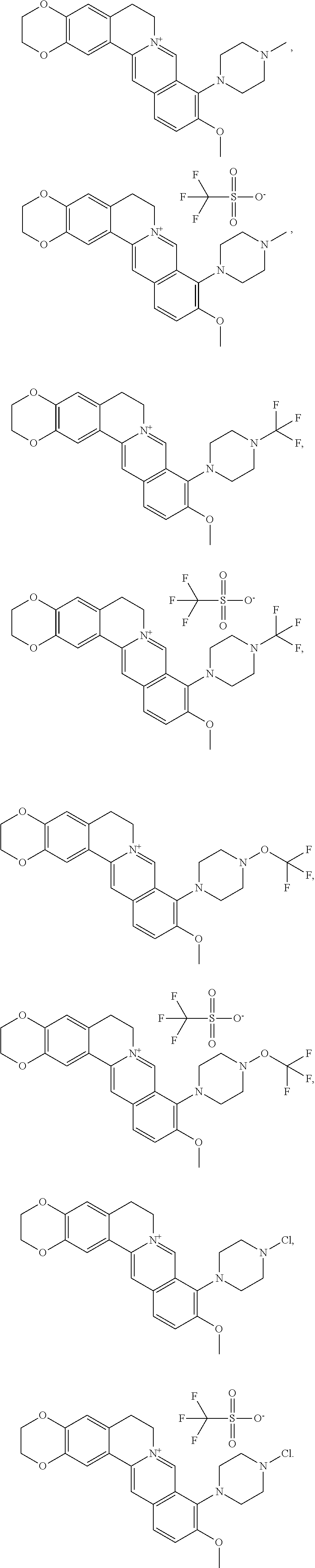

23. A pharmaceutical composition, wherein the pharmaceutical composition comprises (a) the compound of formula I, or an optical isomer thereof, or a racemate thereof, or a solvate thereof, or a pharmaceutically acceptable salt thereof, or a deuterated compound thereof of claim 15; and (b) a pharmaceutically acceptable carrier.

24. The pharmaceutical composition of claim 23, wherein the dosage form of the pharmaceutical composition is a solid preparation, liquid preparation or semi-solid preparation; and/or
the dosage form of the pharmaceutical composition or preparation is oral preparation, external preparation or injection preparation.

25. A method for preventing and/or treating tumor, which comprises administering the compound of formula I, or an optical isomer thereof, or a racemate thereof, or a solvate thereof, or a pharmaceutically acceptable salt thereof, or a deuterated compound thereof of claim 15 to a subject in need, thereby preventing and/or treating tumor.

26. The method of claim 25, wherein the tumor comprises tumor with low expression, no expression, low activity or no activity of mitochondria permeability transition pore; and/or
the tumor comprises tumor with low expression, no expression, low activity or no activity of peptidyl-prolyl cis-trans isomerase F; and/or
the tumor comprises tumor with low or no expression of NNMT gene; and/or
the tumor comprises tumor with high expression of DNA methylase; and/or
the tumor comprises tumor with high expression of UHRF1; and/or
the tumor comprises tumor with high methylation level of nucleotide site of NNMT gene; and/or
the tumor comprises tumor with high methylation level of DNA CpG site of NNMT gene.

27. The method of claim 26, wherein the NNMT gene is human NNMT gene;
the expression comprises protein expression and/or mRNA expression; and/or
the DNA methylase is selected from the group consisting of DNMT1, DNMT3a, DNMT3b, and combinations thereof.

28. The method of claim 26, wherein the low expression or low activity of mitochondria permeability transition pore means the ratio (H1/H0) of the expression level or activity level H1 of mitochondria permeability transition pore in the tumor cell to the expression level or activity level H0 of mitochondria permeability transition pore in the same type of cell is <1.0, preferably ≤0.8, more preferably ≤0.7, more preferably ≤0.6, more preferably ≤0.5, more preferably ≤0.4, more preferably ≤0.3, more preferably ≤0.2, more preferably ≤0.1, more preferably ≤0.05, more preferably ≤0.01, more preferably ≤0.005, more preferably ≤0.001, more preferably ≤0.0001, more preferably ≤0.00001, more preferably ≤0.000001, more preferably ≤0.0000001;
the low expression or low activity of peptidyl-prolyl cis-trans isomerase F means the ratio (P1/P0) of the expression level or activity level P1 of peptidyl-prolyl cis-trans isomerase F in the tumor cell to the expression level or activity level P0 of peptidyl-prolyl cis-trans isomerase F in the same type of cell is <1.0, preferably ≤0.8, more preferably ≤0.7, more preferably ≤0.6, more preferably ≤0.5, more preferably ≤0.4, more preferably ≤0.3, more preferably ≤0.2, more preferably ≤0.1, more preferably ≤0.05, more preferably ≤0.01, more preferably ≤0.005, more preferably ≤0.001, more preferably ≤0.0001, more preferably ≤0.00001, more preferably K 0.000001, more preferably ≤0.0000001;
the low or no expression of NNMT gene means the ratio (E1/E0) of the expression E1 of NNMT gene in the tumor cell to the expression E0 of NNMT gene in the same type of cell is <1.0, preferably ≤0.7, more preferably ≤0.6, more preferably ≤0.5, more preferably ≤0.4, more preferably ≤0.3, more preferably ≤0.2, more preferably ≤0.1, more preferably ≤0.05, more preferably ≤0.01, more preferably ≤0.005, more preferably ≤0.001, more preferably ≤0.0001, more preferably ≤0.00001, more preferably ≤0.000001, more preferably ≤0.0000001;
the tumor with high expression of DNA methylase means the ratio (A1/A0) of the expression level A1 of DNA methylase in the tumor cell to the expression level AG of DNA methylase in the same type of cell is >1.0, preferably ≥1.2, more preferably ≥1.5, more preferably ≥2, more preferably ≥3, more preferably ≥5, more preferably ≥8, more preferably ≥10, more preferably ≥15, more preferably ≥20, more preferably ≥30, more preferably ≥50, such as 2-50;
the tumor with high expression of UHRF1 means the ratio (F1/F0) of the expression level F1 of UHRF1 in the tumor cell to the expression level F0 of UHRF1 in the same type of cell is >1.0, preferably ≥1.2, more preferably ≥1.5, more preferably ≥2, more preferably ≥3, more preferably ≥5, more preferably ≥8, more preferably ≥10, more preferably ≥15, more preferably ≥20, more preferably ≥30, more preferably ≥50, such as 2-50;
the high methylation level of nucleotide site of NNMT gene means the ratio (L1/L0) of the methylation level L1 of nucleotide site of NNMT gene in the tumor cell to the methylation level L0 of nucleotide site of NNMT gene in the same type of cell is >1.0, preferably ≥1.2, more preferably ≥1.5, more preferably ≥2, more preferably ≥3, more preferably ≥5, more preferably ≥8, more preferably ≥10, more preferably ≥15, more preferably ≥20, more preferably ≥30, more preferably ≥50, such as 2-50; and/or
the high methylation level of DNA CpG site of NNMT gene means the ratio (G1/G0) of the methylation level G1 of DNA CpG site of NNMT gene in the tumor cell to the methylation level G0 of DNA CpG site of NNMT gene in the same type of cell is >1.0, preferably ≥1.2, more preferably ≥1.5, more preferably ≥2, more preferably ≥3, more preferably ≥5, more preferably ≥8, more preferably ≥10, more preferably ≥15, more preferably ≥20, more preferably ≥30, more preferably ≥50, such as 2-50.

29. The method of claim 28, wherein the same type of cell comprises the same type of tumor cell with normal expression, high expression, normal activity or high activity of mitochondria permeability transition pore;
the same type of cell comprises the same type of tumor cell with normal expression, high expression, normal activity or high activity of peptidyl-prolyl cis-trans isomerase F;
the same type of cell comprises the same type of tumor cell with normal or high expression of NNMT gene;
the same type of cell comprises the same type of tumor cell with normal or low expression of DNA methylase;
the same type of cell comprises the same type of tumor cell with normal or low expression of UHRF1;
the same type of cell comprises the same type of tumor cell with normal or low methylation level of nucleotide site of NNMT gene; and/or
the same type of cell comprises the same type of tumor cell with normal or low methylation level of DNA CpG site of NNMT gene.

30. The method of claim 26, wherein the methylation level of nucleotide site of NNMT gene refers to the ratio of the number of methylated nucleotides to the number of all nucleotides in the NNMT gene;
the high methylation level of nucleotide site of NNMT gene means the methylation level (M %) of nucleotide site of NNMT gene in the tumor cell is ≥3% and ≤M1%, wherein M1 is any positive integer from 3 to 100;
the high methylation level of nucleotide site of NNMT gene means the methylation level of nucleotide site of NNMT gene in the tumor cell is ≥1%, more preferably ≥3%, more preferably ≥5%, more preferably ≥10%, more preferably ≥15%, more preferably ≥20%, more preferably ≥25%, more preferably ≥30%, more preferably ≥40%, more preferably ≥50%;
the methylation level of nucleotide site of NNMT gene comprises the methylation level of nucleotide site in promoter region of NNMT gene;
the methylation level of nucleotide site of NNMT gene comprises the methylation level of nucleotide sites from 1050 bp before the transcription start site to 499 bp after the transcription start site in NNMT gene;
the methylation level of nucleotide site of NNMT gene comprises the methylation level of nucleotide sites from 1050 bp to 193 bp before the transcription start site in NNMT gene;
the methylation level of nucleotide site of NNMT gene comprises the methylation level of nucleotide sites from 840 bp to 469 bp before the transcription start site in NNMT gene;
the methylation level of nucleotide site of NNMT gene comprises the methylation level of nucleotide site between any two sites (including the two sites itself) selected from group consisting of site 114165695, site 114165730, site 114165769, site 114165804, site 114165938, site 114166050 and site 114166066 on human chromosome 11;
the methylation level of nucleotide site of NNMT gene comprises the methylation level of nucleotide sites selected from group consisting of site 114165695 on human chromosome 11, site 114165730 on human chromosome 11, site 114165769 on human chromosome 11, site 114165804 on human chromosome 11, site 114165938 on human chromosome 11, site 114166050 on human chromosome 11, site 114166066 on human chromosome 11, and combinations thereof;
the methylation level of nucleotide site of NNMT gene comprises the methylation level of nucleotide site between any two sites (including the two sites itself) selected from group consisting of site 1161, site 1196, site 1235, site 1270, site 1404, site 1516 and site 1532 in nucleotide sequence of SEQ ID NO: 1;
the methylation level of nucleotide site of NNMT gene comprises the methylation level of nucleotide sites selected from group consisting of site 1161 in SEQ ID NO: 1, site 1196 in SEQ ID NO: 1, site 1235 in SEQ ID NO: 1, site 1270 in SEQ ID NO: 1, site 1404 in SEQ ID NO: 1, site 1516 in SEQ ID NO: 1, site 1532 in SEQ ID NO: 1, and combinations thereof;
the methylation level of DNA CpG site of NNMT gene refers to the ratio of the number of methylated CpG nucleotides to the number of all nucleotides in the NNMT gene;
the methylation level of DNA CpG site of NNMT gene refers to the ratio of the number of methylated DNA CpG nucleotides to the number of all CpG nucleotides in the NNMT gene;
the high methylation level of DNA CpG site of NNMT gene means the methylation level (M %) of DNA CpG site of NNMT gene in the tumor cell is ≥3% and ≤M2%, wherein M2 is any positive integer from 3 to 100;
the high methylation level of DNA CpG site of NNMT gene means the methylation level of DNA CpG site of NNMT gene in the tumor cell is ≥1%, more preferably ≥3%, more preferably ≥5%, more preferably ≥10%, more preferably ≥15%, more preferably ≥20%, more preferably ≥25%, more preferably ≥30%, more preferably ≥40%, more preferably ≥50%;
the methylation level of DNA CpG site of NNMT gene comprises the methylation level of DNA CpG site in promoter region of NNMT gene;
the methylation level of DNA CpG site of NNMT gene comprises the methylation level of DNA CpG sites from 1050 bp before the transcription start site to 499 bp after the transcription start site in NNMT gene;
the methylation level of DNA CpG site of NNMT gene comprises the methylation level of the DNA CpG sites from 1050 bp to 193 bp before the transcription start site in NNMT gene;
the methylation level of DNA CpG site of NNMT gene comprises the methylation level of DNA CpG sites from 840 bp to 469 bp before the transcription start site in NNMT gene;
the methylation level of DNA CpG site of NNMT gene comprises the methylation level of DNA CpG site between any two sites (including the two sites itself) selected from group consisting of site 114165695, site 114165730, site 114165769, site 114165804, site 114165938, site 114166050 and site 114166066 on human chromosome 11;
the methylation level of DNA CpG site of NNMT gene comprises the methylation level of sites selected from group consisting of site 114165695 on human chromosome 11, site 114165730 on human chromosome 11, site 114165769 on human chromosome 11, site 114165804 on human chromosome 11, site 114165938 on human chromosome 11, site 114166050 on human chromosome 11, site 114166066 on human chromosome 11, and combinations thereof;
the methylation level of DNA CpG site of NNMT gene comprises the methylation level of DNA CpG site between any two sites (including the two sites itself) selected from group consisting of site 1161, site 1196, site 1235, site 1270, site 1404, site 1516 and site 1532 in nucleotide sequence of SEQ ID NO: 1; and/or
the methylation level of DNA CpG site of NNMT gene comprises the methylation level of sites selected from group consisting of site 1161 in SEQ ID NO: 1, site 1196 in SEQ ID NO: 1, site 1235 in SEQ ID NO: 1, site 1270 in SEQ ID NO: 1, site 1404 in SEQ ID NO: 1, site 1516 in SEQ ID NO: 1, site 1532 in SEQ ID NO: 1, and combinations thereof.

31. The method of claim 30, wherein M1 is 5, 10, 15, 20, 25, 30, 35, 40, 45, 50, 55, 60, 65, 70, 80, 85, 90, 95 or 100;
M2 is 5, 10, 15, 20, 25, 30, 35, 40, 45, 50, 55, 60, 65, 70, 80, 85, 90, 95 or 100;
the nucleotide sequence of the promoter region of NNMT gene is as shown in SEQ ID NO: 1;
the sites from 1050 bp before the transcription start site to 499 bp after the transcription start site in NNMT gene is sites 951-2500 of nucleotide sequence as shown in SEQ ID NO: 1;
the sites from 1050 bp to 193 bp before the transcription start site in NNMT gene is sites 951-1808 of nucleotide sequence as shown in SEQ ID NO: 1; and/or
the sites from 840 bp to 469 bp before the transcription start site in NNMT gene is sites 1161-1532 of nucleotide sequence as shown in SEQ ID NO: 1.

32. The method of claim 25, wherein the tumor is human tumor; and/or
the tumor is selected from the group consisting of lung cancer, renal carcinoma, breast cancer, colon cancer, lymphoma, leukemia, pancreatic cancer, brain tumor, liver cancer, prostate cancer, and combinations thereof.

33. The method of claim 32, wherein the lung cancer is selected from the group consisting of non-small cell lung cancer, small cell lung cancer, and combinations thereof;
the colon cancer comprises colon adenocarcinoma;
the breast cancer comprises triple negative breast cancer;
the lymphoma is selected from the group consisting of B-cell lymphoma, skin T-cell lymphoma, and combinations thereof;
the brain tumor is selected from the group consisting of brain glioblastoma, neuroglioma, brain medulloblastoma, brain neuroblastoma, and combination thereof;
the renal carcinoma is selected from the group consisting of clear cell renal cell adenocarcinoma, renal carcinoma Wilms, and combination thereof; and/or
the leukemia is selected from the group consisting of T-lymphocyte leukemia, myeloid leukemia, and combinations thereof.

34. The method of claim 33, wherein the lymphoma comprises diffuse large B-cell lymphoma;
the brain medulloblastoma comprises cerebellar medulloblastoma;
the brain glioblastoma comprises glioblastoma multiforme;
the T-lymphocytic leukemia comprises acute T-lymphocytic leukemia;
the myeloid leukemia comprises type M4 of acute myeloid leukemia; and/or
the myeloid leukemia comprises FAB type M4 of acute myeloid leukemia.
